(19) United States
(12) Patent Application Publication
(10) Pub. No.: US 2023/0303974 A1
(43) Pub. Date: Sep. 28, 2023

(54) Immune Cells Defective for SOCS1

(71) Applicants: Institut Curie, Paris (FR); Institut National de la Santé et de la Recherche Médicale, Paris (FR)

(72) Inventors: Laurie Menger, Vitry sur Seine (FR); Silvia Menegatti, Paris (FR); Sebastian Amigorena, Paris (FR)

(73) Assignees: Institut Curie, Paris (FR); Institut National de la Santé et de la Recherche Médicale, Paris (FR)

(21) Appl. No.: 18/018,635

(22) PCT Filed: Jul. 30, 2021
(86) PCT No.: PCT/EP2021/071504

§ 371 Date: Jan. 30, 2023

(30) Foreign Application Priority Data
Jul. 30, 2020 (EP) 20305878.9

(51) Int. Cl. C12N 5/0783; C07K 14/725; A61K 35/17; A61P 35/00

(52) U.S. Cl. CPC C12N 5/0636; C07K 14/7051; A61K 35/17; A61P 35/00

(57) ABSTRACT

The present invention relates to an engineered immune cell defective for SOCS1. Preferably, said engineered immune cell further comprises a genetically engineered antigen receptor that specifically binds a target antigen. The present invention also relates to a method for obtaining a genetically engineered immune cell comprising a step consisting in inhibiting the expression and/or activity of SOCS1 in the immune cell; and further optionally comprising a step consisting in introducing in the said immune cell a genetically engineered antigen receptor that specifically binds to a target antigen. The invention also encompasses said engineered immune cell for their use in adoptive therapy, notably for the treatment of cancer.

### FIELD OF THE INVENTION

The present invention relates to the field of adoptive therapy. The present invention provides immune cells defective for SOCS1 with enhanced expansion, survival, and functionality in vivo.

### INTRODUCTION

Adoptive T cell therapy (ATCT), including T cells engineered with recombinant T Cell Receptor (TCR) or Chimeric Antigen Receptor (CAR) or tumor-infiltrating lymphocytes (TILs), is emerging as a powerful cancer therapy.

The in vitro manufacturing process enables to genetically reprogram a heterogenous mixture of CD4 and CD8 T cell live drug with complex sensing-response behaviors (Lim et June 2017). Although CD8 or CD4 T cells alone can exert significant therapeutic effects (Freitas et Rocha 2000), the co-injection of both subsets is often a crucial requirement for an optimal and sustained antitumor activity (Linnemann, Schumacher, et Bendle 2011; Sadelain 2015; Borst et al. 2018). Exhibiting pleiotropic effects and plasticity, CD4 T cells can boost antitumor immune responses through both helper (Corthay et al. 2005; Bos et Sherman 2010; Z. Zhu et al. 2015) and cytotoxic functions (Xie et al. 2010; Quezada et al. 2010; Kitano et al. 2013; Śledzińska et al. 2020a).

However, activated CD4 and CD8 T cells differ in their capacity to proliferate and persist in vivo. While CD8 T cells undergo extensive and autonomous clonal expansion, CD4 T cells need repeated antigen triggering and exhibit proliferative arrest in early divisions leading to approximately 10-20 fold less expansion (Homann, Teyton, et Oldstone 2001; Foulds et al. 2002; Seder et Ahmed 2003; Ravkov et Williams 2009).

Discrepancies in the magnitude and duration of CD4 and CD8 T-cell expansion are not due to external signals nor competition for resources (see above references). Instead, by comparing the entry of naive and antigen-experienced (Ag-exp) CD4 T cells into an immune response following antigen stimulations, several studies reported that Ag-exp CD4 T cells specifically curtail their own proliferation and exhibit a reduced IL2 production (Foulds et al. 2002; Merica et al. 2000; MacLeod, Kappler, et Marrack 2010; Helft et al. 2008). The inventors have previously developed an in vivo model reproducing the dysfunctional expansion of Ag-exp CD4 T-cell during an ongoing immune response. In this physiologically relevant model, generalizable to several CD4 TCR-transgenic (Tg) T cells, Ag-exp CD4 T-cell expansion is intrinsically abolished, while naive CD4 T cell proliferation is maintained, demonstrating that the absence of Ag-exp CD4 proliferation is not related to insufficient priming (Helft et al. 2008). The strong inhibition that they previously reported is Ag specific, begins at day 2 (long before Ag disappearance) and is neither due to extrinsic factors, such as regulatory T cells (Tregs), lack of antigen presenting cell (APCs) education nor competition for Ag (Helft et al. 2008). Instead, they showed that Ag-exp CD4 T cells are stopped by an intrinsic, active and dominant phenomenon, which cannot be overcome by providing new Ag-loaded DCs.

In a context of Adoptive T Cell Therapy (ATCT), where T cells are activated in vitro prior to the engineering process, Ag-exp CD4 T-cells can become a limiting subset under recall conditions in vivo, compromising an efficient protective immune response (Homann, Teyton, et Oldstone 2001). The underlying molecular mechanisms involved in this limited expansion are unknown but can interfere with ATCT efficacy, as small doses of T cells are infused into patients.

Thus, there remains a need for engineered immune cells, notably engineered T cells, exhibiting enhanced expansion capability and survival after adoptive transfer. There is also still a need for engineered T cells with improved functional efficacy, in particular with improved cytotoxic potential, which would support efficient and broad scale cancer treatment.

Furthermore, the manufacturing of autologous T cells is tedious, expensive and often inefficient. To ensure the stable establishment of ATC therapies as a drug, well-characterized, banked and premanufactured therapeutic cells from healthy donors will address these limitations. Therefore, efforts to develop potent allogeneic T cells that are not rejected by the recipient's immune system meet an important clinical need.

Hence, the inventors are investigating the intrinsic resistance of T cells to host immune elimination, with the aim of generating universal cell therapies from healthy donors. Although the use of autologous therapeutic CAR T-cell has resulted to date in outstanding clinical data (Neelapu et al. 2017; Maude et al. 2018), it has certain well-known disadvantages.

First, the complex personalized manufacturing reduces their scalability (Graham et al. 2018). In addition, the current manufacturing processes take approximately 3 weeks (Kohl et al. 2018), which limits their availability, especially for patients with highly proliferative diseases (Depil et al. 2020). Finally, autologous T cells potency can be negatively impacted by previous lines of treatment or immunosuppression derived from the tumor microenvironment (Thommen et Schumacher 2018).

Conversely, while the use of allogeneic T-cell product (HLA-mismatched) from healthy donors allows the immediate access to standardized batches of T cells, improving their efficacy (multiple cell modifications, combination of targets), decreasing their cost and industrialized process (Lin et al. 2019), it is nevertheless associated with two major difficulties. First, the transfer of allogeneic T cells can cause a life-threatening disease, the graft versus host disease (GVHD) induced by donor T-lymphocytes. One strategy to prevent it, is the genetic inactivation of TCRα constant (TRAC) gene. Second, TCR-negative allogeneic T cells can still be non-self HLA-recognized and rapidly eliminated by the host's immune system, which will limit their anti-tumor activity. On this regard, lymphodepletion with chemotherapy or irradiation before universal CAR-T cells infusion have been proposed to delay the rejection until the recipient immune system recovers (Gattinoni et al. 2005) but they are associated with significant toxicities and problematic viral reactivations (Chakrabarti, Hale, et Waldmann 2004).

Since HLA-I molecules are the key mediators of immune rejection, another proposed strategy was the genetic disruption of β2-microglobulin, which is essential for forming functional HLA class I molecules on the cell surface (Poirot et al. 2015; D. Wang et al. 2015; Torikai et al. 2013). However, these cells may become the target of NK cells that are sensitive to reduced HLA expression (missing-self mechanism) (Bern et al. 2019). Solutions to prevent to NK-mediated rejection could rely on the overexpression of HLA-E molecules (Gornalusse et al. 2017), ligand of the inhibitory complex CD94/NGK2A (Braud et al. 1998) or HLA-G, normally expressed by cytotrophoblasts, binding to the inhibitory receptors KIR2DL4/IT2 (Rajagopalan et Long 1999; Pazmany et al. 1996; Gonen-Gross et al. 2010).

Finally, using the hypoimmunogenic cells induced Pluripotent Stem (iPS), combined with CAR technology can also provide a promising and unlimited source of lymphocytes with antigenic specificity and independence from HLA restriction (Themeli et al. 2013). However, difficulties persist, particularly with the differentiation methods which are not currently good manufacturing practice (GMP)-compatible since they include the presence of serum and murine derived feeder cells. Also, as it involves a multi-step differentiation process, the developmental transition can occur with different efficiency.

Therefore, the production of mature single positive T cells for clinical applications, notably for allogenic transfer, remains a challenge (Nianias et Themeli 2019).

### SUMMARY OF THE INVENTION

The inventors have developed a strategy to genetically manipulate primary T cells at the genome-wide (GW) level using CRISPR technology. This innovative approach allows rapid, systematic and unbiased identification of T-cell intrinsic limiting factors, functionally non-redundant in vivo (13,14). First, the inventors have interrogated intrinsic factors limiting rechallenged CD4 T cells expansion in vivo. Their screens identified Suppressor of Cytokine Signaling 1 (SOCS1) as a non-redundant and intrinsic inhibitor of CD4+ T-cell proliferation and survival. They demonstrated that SOCS1 is a critical node, integrating cytokines signals (IFN-γ and IL-2) to actively limit CD4+ T cell functions. The inventors investigated the function of SOCS1 in both mouse and human CD4+ and CD8+ antitumor adoptive cell therapies. SOCS1 inactivation restored CD4+T helper-1 (Th1) cells expansion, as well as cytotoxic functions whereas in CD8+ T cell it greatly boosted cytotoxic potential.

Then, using a similar genome-wide screening strategy in vivo and by transferring a pool of genome-wide mutated Marilyn T from C57BL6 mice (H2-Kb) in fully immunocompetent BALB/c (H2-Kd) MHC-mismatched mice, the inventors have re-identified P2m, an empirical target whose reduced expression coincide with immune evasion in nature (Lanza, Russell, et Nagy 2019), particularly in cancers (Koopman et al. 2000; He et al. 2017). Moreover and fully unexpectedly, the inventors have identified Fas (CD95, Tnfrsf6) that they now validate as a major target improving the survival of allogenic T cells in vivo. These results represent a major advance for the development of universal (allogenic) cell therapy based on allogenic transplantation (such as immune cell therapy).

The inventors further provide results supporting that the combination of SOCS1 and FAS-inactivations provide a “fratricide/allogeneic death resistant” universal T-cell product.

Therefore, the present invention relates to modified, or engineered, immune cells, notably modified T cells, wherein SOCS-1 is inactivated. In some embodiments, the said immune cell is also defective for FAS and/or Suv39h1.

Typically, the engineered immune cell of the present application 1 is a T cell or an NK cell. More particularly, the T cell is a CD4+ or CD8+ T cell. Preferred cells may be selected from Naïve T cells (T_{N} cells), Stem memory T cells (TSC_{M} cells), memory T cells (TC_{M} cells), tumor-infiltrating lymphocytes (TILs), or effector memory T cells (TE_{M} cells) and combination thereof.

Typically also, the engineered immune cell is isolated from a subject. Preferably, said subject is suffering from a cancer, or is at risk of suffering from a cancer.

The target antigen to which the genetically engineered antigen receptor specifically binds is preferably expressed on cancer cells and/or is a universal tumor antigen.

The genetically engineered antigen receptor can be a chimeric antigen receptor (CAR) comprising an extracellular antigen-recognition domain that specifically binds to the target antigen. The genetically engineered antigen receptor can also be a T cell receptor (TCR).

Preferably, the activity and/or expression of SOCS-1 and in some embodiments of also FAS and/or Suv39h1 in the said engineered immune cell is selectively inhibited or blocked. In one embodiment, said engineered immune cell expresses a SOCS-1, a FAS or a Suv39h1 nucleic acid encoding a non-functional SOCS-1, FAS or Suv39h1 protein respectively.

The present application also relates to a method of producing a genetically engineered immune cell notably a universal immune cell (usable in allogenic transplantation, in particular in allogenic adoptive cellular therapy) comprising a step consisting in inhibiting the expression and/or activity of SOCS-1 and/or FAS and in some embodiments further inhibiting the expression and/or activity of β2m and/or Suvh39h1 in the immune cell; and optionally a step consisting in introducing into an immune cell a genetically engineered antigen receptor that specifically binds to a target antigen.

In some embodiments, the inhibition of SOCS-1, FAS, Suv39h1, or β2m activity and/or expression comprises contacting, or putting in contact, the cell with at least an agent inhibiting the expression and/or activity of SOCS-1, FAS, Suv39h1 or β2m protein(s) and/or disrupting the FAS, β2m SOCS-1 and/or Suv39h1 gene(s). Said agent can be selected from small molecule inhibitors; antibodies derivatives, aptamers, nucleic acid molecules that block transcription or translation, or gene editing agents targeting respectively SOCS1, FAS, Suv39h1 or B2N gene.

The present invention also refers to an engineered immune cell as described herein, or a composition comprising said engineered immune cell, for use in adoptive cellular therapy, notably adoptive therapy of cancer.

### DETAILED DESCRIPTION

### Definitions

The term “antibody” herein is used in the broadest sense and includes polyclonal and monoclonal antibodies, including intact antibodies and functional (antigen-binding) antibody fragments, including fragment antigen binding (Fab) fragments, F(ab′)2 fragments, Fab′ fragments, Fv fragments, recombinant IgG (rIgG) fragments, variable heavy chain (VH) regions capable of specifically binding the antigen, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; variable heavy chain (VH) regions, single-chain antibody molecules such as scFvs and single-domain VH single antibodies; and multispecific antibodies formed from antibody fragments. In particular embodiments, the antibodies are single-chain antibody fragments comprising a variable heavy chain region and/or a variable light chain region, such as scFvs.

“Single-domain antibodies” are antibody fragments comprising all or a portion of the heavy chain variable domain or all or a portion of the light chain variable domain of an antibody. In certain embodiments, a single-domain antibody is a human single-domain antibody.

As used herein, “repression” of gene expression refers to the elimination or reduction of expression of one or more gene products encoded by the subject gene in a cell, compared to the level of expression of the gene product in the absence of the repression. Exemplary gene products include mRNA and protein products encoded by the gene. Repression in some cases is transient or reversible and in other cases is permanent. Repression in some cases is of a functional or full-length protein or mRNA, despite the fact that a truncated or non-functional product may be produced. In some embodiments herein, gene activity or function, as opposed to expression, is repressed. Gene repression is generally induced by artificial methods, i.e., by addition or introduction of a compound, molecule, complex, or composition, and/or by disruption of nucleic acid of or associated with the gene, such as at the DNA level. Exemplary methods for gene repression include gene silencing, knockdown, knockout, and/or gene disruption techniques, such as gene editing. Examples include antisense technology, such as RNAi, siRNA, shRNA, and/or ribozymes, which generally result in transient reduction of expression, as well as gene editing techniques which result in targeted gene inactivation or disruption, e.g., by induction of breaks and/or homologous recombination.

As used herein, a “disruption” of a gene refers to a change in the sequence of the gene, at the DNA level. Examples include insertions, mutations, and deletions. The disruptions typically result in the repression and/or complete absence of expression of a normal or “wild type” product encoded by the gene. Exemplary of such gene disruptions are insertions, frameshift and missense mutations, deletions, knock-in, and knock-out of the gene or part of the gene, including deletions of the entire gene. Such disruptions can occur in the coding region, e.g., in one or more exons, resulting in the inability to produce a full-length product, functional product, or any product, such as by insertion of a stop codon. Such disruptions may also occur by disruptions in the promoter or enhancer or other region affecting activation of transcription, so as to prevent transcription of the gene. Gene disruptions include gene targeting, including targeted gene inactivation by homologous recombination.

### Cells of the Invention

The cells according to the invention are typically eukaryotic cells, such as mammalian cells (also named in the present invention animal cells), e.g., human cells.

More particularly, the cells of the invention are derived from the blood, bone marrow, lymph, or lymphoid organs (notably the thymus) and are cells of the immune system (i.e., immune cells), such as cells of the innate or adaptive immunity, e.g., myeloid or lymphoid cells, including lymphocytes, typically T cells and/or NK cells.

Preferably according to the invention, cells are notably lymphocytes including T cells, B cells and NK cells.

Cells according to the invention may also be immune cell progenitors, such as lymphoid progenitors and more preferably T cell progenitors.

T cell progenitors typically express a set of consensus markers including CD44, CD117, CD135, and Sca-1 but see also Petrie H T, Kincade P W. Many roads, one destination for T cell progenitors. The Journal of Experimental Medicine. 2005; 202(1):11-13.

The cells typically are primary cells, such as those isolated directly from a subject and/or isolated from a subject and frozen.

With reference to the subject to be treated, the cells of the invention may be allogeneic and/or autologous.

In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation.

Among the sub-types and subpopulations of T cells and/or of CD4+ and/or of CD8+ T cells are naive T (T_{N}) cells, effector T cells (T_{EFF}), memory T cells and sub-types thereof, such as stem cell memory T (TSC_{M}), central memory T (TC_{M}), effector memory T (T_{EM}), or terminally differentiated effector memory T cells, tumor-infiltrating lymphocytes (TILs), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells. Preferably, the cells according to the invention are T_{EFF} cells with stem/memory properties and higher reconstitution capacity due to the inhibition of Suv39h1, as well as T_{N} cells, TSC_{M}, TC_{M}, TE_{M} cells and combinations thereof.

In some embodiments, one or more of the T cell populations is enriched for, or depleted of, cells that are positive for or express high levels of one or more particular markers, such as surface markers, or that are negative for or express relatively low levels of one or more markers. In some cases, such markers are those that are absent or expressed at relatively low levels on certain populations of T cells (such as non-memory cells) but are present or expressed at relatively higher levels on certain other populations of T cells (such as memory cells). In one embodiment, the cells (such as the CD8⁺ cells or the T cells, e.g., CD3⁺ cells) are enriched for (i.e., positively selected for) cells that are positive or expressing high surface levels of CD117, CD135, CD45RO, CCR7, CD28, CD27, CD44, CD127, and/or CD62L and/or depleted of (e.g., negatively selected for) cells that are positive for or express high surface levels of CD45RA. In some embodiments, cells are enriched for or depleted of cells positive or expressing high surface levels of CD122, CD95, CD25, CD27, and/or IL7-Ra (CD127). In some examples, CD8+ T cells are enriched for cells positive for CD45RO (or negative for CD45RA) and for CD62L.

For example, according to the present application, the cells can include a CD4+ T cell population and/or a CD8+ T cell sub-population, e.g., a sub-population enriched for central memory (T_{CM}) cells. Alternatively, the cells can be other types of lymphocytes, including natural killer (NK) cells, MAIT cells, Innate Lymphoid Cells (ILCs) and B cells.

The cells and compositions containing the cells for engineering according to the invention are isolated from a sample, notably a biological sample, e.g., obtained from or derived from a subject. Typically, the subject needs a cell therapy (adoptive cell therapy) and/or will receive the cell therapy. The subject is preferably a mammal, notably a human. In one embodiment of the present application, the subject has a cancer.

The samples include tissues, fluids, and other samples taken directly from the subject, as well as samples resulting from one or more processing steps, such as separation, centrifugation, genetic engineering (for example transduction with viral vector), washing, and/or incubation. Therefore, the biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom. Preferably, the sample from which the cells are derived or isolated is blood or a blood-derived sample or is or, is derived from, an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, and/or cells derived therefrom. Samples include, in the context of cell therapy (typically adoptive cell therapy) samples from autologous and allogeneic sources.

In some embodiments, the cells are derived from cell lines, e.g., T cell lines. The cells can also be obtained from a xenogeneic source, such as a mouse, a rat, a non-human primate, or a pig. Preferably, the cells are human cells.

### Cells Defective for SOCS-1

The present disclosure encompasses cells, more specifically immune cells defective for SOCS1. In some embodiments, SOCS1 defective cells can be further defective for FAS, β2m, SUV39h1 or combination thereof.

As used herein the term “SOCS-1” or “Suppressor of cytokine signaling 1” has its general meaning in the art and is part to the SOCS family proteins which form part of a classical negative feedback system that regulates cytokine signal transduction. There are eight SOCS proteins encoded in the human genome, SOCS1-7 and CIS. All eight are defined by the presence of an SH2 domain and a short, C-terminal domain, the SOCS box1. The SOCS box of all SOCS proteins are found associated with an adapter complex, elongin B,C. This association allows recruitment of an E3 ubiquitin ligase scaffold (Cullin5) to catalyze the ubiquitination of signaling intermediates recruited by their SH2 domains (Kamizono S et al., “The SOCS box of SOCS-1 accelerates ubiquitin-dependent proteolysis of TEL-JAK2”. J Biol Chem. 2001 Apr. 20; 276(16):12530-8).

In addition to their ubiquitin ligase activity, SOCS1 and SOCS3 are unique in also having the ability to directly inhibit the kinase activity of JAK (Janus Kinases). This activity relies upon a short motif, which is immediately upstream of the SH2 domain, known as the KIR (kinase inhibitory region). The KIR of SOCS1 is a highly evolved inhibitor of JAK and mutation of any residue within this motif, including the histidine residue that mimics the substrate tyrosine, leads to a significant decrease in affinity. SOCS1 is in particular a direct, potent and selective inhibitor of notably JAK1 and JAK2 as well as TYK2 catalytic activity and thus is typically involved in negative regulation of a number of cytokines, including interleukin-4 (IL-4), IL-6, IL-2, interferon (IFN)-alpha, interferon (IFN)-gamma, prolactin, growth hormone, and erythropoietin, that signal through the JAK/STAT3 pathway. (see notably for details on SOCS1 activity: Sharma J, Larkin J 3rd. “Therapeutic Implication of SOCS1 Modulation in the Treatment of Autoimmunity and Cancer”. Front Pharmacol. 2019; 10:324; Liau N P D, Laktyushin A, Lucet I S, et al. “The molecular basis of JAK/STAT inhibition by SOCS1”. Nat Commun. 2018; 9(1):1558); Sporri B, Kovanen P E, Sasaki A, Yoshimura A, Leonard W J. “JAB/SOCS1/SSI-1 is an interleukin-2-induced inhibitor of IL-2 signaling”. Blood. 2001; 97(1):221-226; Alexander W S, Starr R, Fenner J E, et al. “SOCS1 is a critical inhibitor of interferon gamma signaling and prevents the potentially fatal neonatal actions of this cytokine”. Cell. 1999; 98(5):597-608 as well as Kamizono S, Hanada T, Yasukawa H, et al. “The SOCS box of SOCS-1 accelerates ubiquitin-dependent proteolysis of TEL-JAK2.” J Biol Chem. 2001; 276(16):12530-12538; and Frantsve J, Schwaller J, Sternberg D W, Kutok J, Gilliland D G. “Socs-1 inhibits TEL-JAK2-mediated transformation of hematopoietic cells through inhibition of JAK2 kinase activity and induction of proteasome-mediated degradation.” Mol Cell Biol. 2001; 21(10):3547-3557). This protein is also known as JAK-binding protein (JAB), STAT-induced STAT inhibitor 1 (SSI-1) or Tec-interacting protein 3 (TIP-3). The human SOCS-1 protein is referenced 015524 in UNIPROT, and is encoded by the gene SOCS-1 located on chromosome 16 (11,254,408-11,256,204 reverse strand.) and referenced as ENSG00000185338 in the Ensembl database. The term SOCS-1 also encompasses all SOCS-1 orthologs. In some embodiments, the protein SOCS-1 according to the present invention is of SEQ ID NO:1: MVAHNQVAADNAVSTAAEPRRRPEPSSSSSSSPAAPARPRPCPAVPAPAPGDTHFRT FRSHADYRRITRASALLDACGFYWGPLSVHGAHERLRAEPVGTFLVRDSRQRNCFFA LSVKMASGPTSIRVHFQAGRFHLDGSRESFDCLFELLEHYVAAPRRMLGAPLRQRRV RPLQELCRQRIVATVGRENLARIPLNPVLRDYLSSFPFQIAs used herein the expression “defective for SOCS1” according to the present invention refers to the inhibition, or blockade of SOCS-1 activity, such as for example the blockage of the binding of SOCS1 on JAK and/or the blockage of the recruitment of an E3 ubiquitin ligase scaffold (Cullin5) through elonginBC. In some embodiments, inhibition of SOCS1 may be obtained by preventing the binding of SOCS1 on the JAKs (including JAK1/2 and/or TYK2), and/or by preventing prevent the SOCS1 Box from binding to Elongin C, an important intermediate of E3 complex recruitment.

As also used herein the term “Suv39h1” or “H3K9-histone methyltransferase Suv39h1” has its general meaning in the art and refers to the histone methyltransferase “suppressor of variegation 3-9 homolog 1 (Drosophila)” that specifically trimethylates the Lys-9 residue of histone H3 using monomethylated H3-Lys-9 as substrate (see also Aagaard L, Laible G, Selenko P, Schmid M, Dorn R, Schotta G, Kuhfittig S, Wolf A, Lebersorger A, Singh P B, Reuter G, Jenuwein T (June 1999). “Functional mammalian homologues of the Drosophila PEV-modifier Su(var)3-9 encode centromere-associated proteins which complex with the heterochromatin component M3 1″. EMBO J 1 8 (7): 1923-38). Said histone methyltransferase is also known as MG44, KMT1A, SUV39H, SUV39H1, histone-lysine N-methyltransferase SUV39H1, H3-K9-HMTase 1, OTTHUMP00000024298, Su(var)3-9 homolog 1, lysine N-methyltransferase 1A, histone H3-K9 methyltransferase 1, position-effect variegation 3-9 homolog, histone-lysine N-methyltransferase, or H3 lysine-9 specific 1. The human Suv39h1 methyltransferase is referenced 043463 in UNIPROT and is encoded by the gene Suv39h1 located on chromosome x (gene ID: 6839 in NCBI) The term Suv39h1 according to the invention also encompasses all orthologs of SUV39H1 such as SU(VAR)3-9. In some embodiments, the protein SUV39H1 according to the present invention is of SEQ ID NO:2 or 3.

SEQ ID NO: 2:
MAENLKGCSVCCKSSWNQLQDLCRLAKLSCPALGISKRNLYDFEVEYLCD
YKKIREQEYYLVKWRGYPDSESTWEPRQNLKCVRILKQFHKDLERELLRR
HHRSKTPRHLDPSLANYLVQKAKQRRALRRWEQELNAKRSHLGRITVENE
VDLDGPPRAFVYINEYRVGEGITLNQVAVGCECQDCLWAPTGGCCPGASL
HKFAYNDQGQVRLRAGLPIYECNSRCRCGYDCPNRVVQKGIRYDLCIFRT
DDGRGWGVRTLEKIRKNSFVMEYVGEIITSEEAERRGQIYDRQGATYLFD
LDYVEDVYTVDAAYYGNISHFVNHSCDPNLQVYNVFIDNLDERLPRIAFF
ATRTIRAGEELTFDYNMQVDPVDMESTRMDSNFGLAGLPGSPKKRVRIEC
KCGTESCRKYLF
SEQ ID NO: 3:
MVGMSRLRNDRLADPLTGCSVCCKSSWNQLQDLCRLAKLSCPALGISKRN
LYDFEVEYLCDYKKIREQEYYLVKWRGYPDSESTWEPRQNLKCVRILKQF
HKDLERELLRRHHRSKTPRHLDPSLANYLVQKAKQRRALRRWEQELNAKR
SHLGRITVENEVDLDGPPRAFVYINEYRVGEGITLNQVAVGCECQDCLWA
PTGGCCPGASLHKFAYNDQGQVRLRAGLPIYECNSRCRCGYDCPNRVVQK
GIRYDLCIFRTDDGRGWGVRTLEKIRKNSFVMEYVGEIITSEEAERRGQI
YDRQGATYLFDLDYVEDVYTVDAAYYGNISHFVNHSCDPNLQVYNVFIDN
LDERLPRIAFFATRTIRAGEELTFDYNMQVDPVDMESTRMDSNFGLAGLP
GSPKKRVRIECKCGTESCRKYLF

As used herein, the term “Fas” or “Fas Cell Surface Death Receptor” has its general meaning in the art and refers to the receptor for TNFSF6/FASLG. Also known as Fas receptor (FasR), apoptosis antigen 1 (APO-1 or APT), cluster of differentiation 95 (CD95) or tumor necrosis factor receptor superfamily member 6 (TNFRSF6), Fas is a protein that in humans is encoded by the FAS gene. FAS is a death receptor located on the surface of cells that leads to programmed cell death (apoptosis) if it binds its ligand, Fas ligand (FasL), thus forming the death-inducing signaling complex (DISC) and inducing subsequent caspase 8 activation, via the adaptor molecule FADD. It is one of two apoptosis pathways, the other being the mitochondrial pathway. The human Fas is referenced as β25445 (TNR6_HUMAN) in UNIPROT and is encoded by the gene FAS located on chromosome 10 (88,990,531-89,017,059 forward strand), referenced as ENSG00000026103 in Ensembl database. The term FAS also encompasses all FAS1 orthologs.

In some embodiments, the protein FAS as herein intended is of SEQ ID NO:4

SED ID NO: 4:
MLGIWTLLPLVLTSVARLSSKSVNAQVTDINSKGLELRKTVTTVETQNLE
GLHHDGQFCHKPCPPGERKARDCTVNGDEPDCVPCQEGKEYTDKAHFSSK
CRRCRLCDEGHGLEVEINCTRTQNTKCRCKPNFFCNSTVCEHCDPCTKCE
HGIIKECTLTSNTKCKEEGSRSNLGWLCLLLLPIPLIVWVKRKEVQKTCR
KHRKENQGSHESPTLNPETVAINLSDVDLSKYITTIAGVMTLSQVKGFVR
KNGVNEAKIDEIKNDNVQDTAEQKVQLLRNWHQLHGKKEAYDTLIKDLKK
ANLCTLAEKIQTIILKDITSDSENSNFRNEIQSLV

Beta-2-microglobulin (β2m) is a component of the class I major histocompatibility complex (MHC). Involved in the presentation of peptide antigens to the immune system. The human β2m is encoded by the B2M gene with chromosomal location 15q21.1 (Chromosome 15: 44,711,487-44,718,851 forward strand), referenced B2M ENSG00000166710 in Ensembl database (or HGNC ID: HGNC:914). β2m precursor is typically of SEQ US NO:5 which is further processed in the mature form.

MSRSVALAVLALLSLSGLEAIQRTPKIQVYSRHPAENGKSNFLNCYVSGF
HPSDIEVDLLKNGERIEKVEHSDLSFSKDWSFYLLYYTEFTPTEKDEYAC
RVNHVTLSQPKIVKWDRDM

As used herein the expressions “defective for SOCS1”, “defective for Suv39h1”, “defective for FAS”, or defective for β2m according to the present application refers to the inhibition, or blockade of SOCS1, and/or Suv39h1 and/or FAS activity and/or β2m activity, as detailed above, in the cell.

“Inhibition of SOCS1 activity” or “inhibition of Suv39h1 activity” or “inhibition of FAS activity” or “inhibition of β2m activity” as intended in the present application refers to a decrease of SOCS1 activity, of Suv39h1 of FAS, or of β2m activity of at least 30%, 40%, 50%, 60%, 70%, 80%, 90%, 95% or 99% or more as compared to the activity, or level, of the SOCS1, Suv39h1 or FAS protein which is not inhibited in a corresponding wild-type cell. Preferentially, the inhibition of SOCS1 activity of Suv39h1, or of FAS activity leads to the absence in the cell of substantial detectable activity of SOCS1, Suv39h1, or FAS respectively.

It is to be noticed that a cell defective for SOCS1, and/or Suv39h1 and/or FAS and/or β2m can be obtained by repression or disruption of respectively the SOCS1, and/or Suv39h1 and/or FAS and/or B2M gene(s), but also at the post-transcriptional level (SOCS1 mRNA, and/or Suv39h1 and/or FAS mRNA and/or β2m mRNA) as well at the post-translational or protein level of SOCS1, and/or FAS and/or Suv39h1 and/or β2m.

Inhibition of SOCS1, and/or FAS and/or Suv39h1 and/or β2m activity can thus also be achieved through repression of SOCS1, and/or FAS and/or Suv39h1 and/or β2m gene expression or though SOCS1 and/or FAS and/or Suv39h1 and/or B2M gene disruption. According to the invention, said repression reduces expression of SOCS1 and/or FAS and/or Suv39h1 and/or β2m in the cell, notably the immune cell of the invention by at least 50, 60, 70, 80, 90, or 95% as to the same cell (i.e. corresponding cell) produced by the method in the absence of the repression or in corresponding wild-type cell (as illustrated in the results included herein). Gene disruption may also lead to a reduced expression of the SOCS1 and/or FAS and/or Suv39h1 and/or β2m protein or to the expression of a non-functional SOCS1 protein, and/or a non-functional FAS protein and/or of a non-functional Suv39h1 protein and/or of a non-functional β2m protein.

By “non-functional” SOCS1 protein”, “non-functional” FAS protein”, “non-functional” Suv39h1 protein, or “non-functional β2m protein” it is herein intended a protein with a reduced activity or a lack of detectable activity as described above.

In some embodiments, inhibitors of SOCS1 activity in a cell according to the invention can be selected among any compound or agent natural or not having the ability of preventing binding of SOCS1 to JAK and/or Elongin C, or inhibiting the SOCS1 gene expression. Inhibitors of SOCS1 activity in a cell according to the invention can be selected among any compound or agent natural or not having the ability of inhibiting SOCS1 activity, notably as above mentioned, or inhibiting the SOCS1 gene expression.

In some embodiments, a peptide mimetic of SOCS1 or the autophosphorylation site pJAK2 (1001-1013) as described in Lilian W Waiboci, Howard M Johnson, James P Martin and Chulbul M Ahmed, J Immunol Apr. 1, 2007, 178 (1 Supplement) S170; or in Waiboci L W, Ahmed C M, Mujtaba M G, et al. J Immunol. 2007; 178(8):5058-5068, can be used

In some embodiments, inhibitors of FAS activity in a cell according to the invention can be selected among any compound or agent natural or not having the ability of preventing the FAS receptor activation or inhibiting the FAS gene expression.

In some embodiments, inhibitors of Suv39h1 activity in a cell according to the invention can be selected among any compound or agent natural or not having the ability of inhibiting the methylation of Lys-9 of histone H3 by H3K9-histone methyltransferase, or inhibiting the H3K9-histone methyltransferase SUV39H1 gene expression. Inhibitors of Suv39h1 activity in a cell according to the invention can be selected among any compound or agent natural or not having the ability of inhibiting the methylation of Lys-9 of histone H3 by H3K9-histone methyltransferase, or inhibiting the H3K9-histone methyltransferase SUV39H1 gene expression.

Inhibition of SOCS1 and/or FAS and/or Suv39h1 and/or β2m (at the gene and/or protein level) in the immune cell according to the present application can be permanent and irreversible or transient or reversible. Preferably however, SOCS1 inhibition and/or FAS inhibition and/or Suv39h1 inhibition is/are permanent and irreversible. Inhibition of SOCS1 and/or FAS and or of Suv39h1 in the cell may be achieved prior or after injection of the cell in the targeted patient as described below.

### Genetically Engineered Cells According to the Invention

In some embodiments, the cells comprise one or more nucleic acids introduced via genetic engineering that encode one or more antigen receptors.

Typically, the nucleic acids are heterologous, (i.e., for example which are not ordinarily found in the cell being engineered and/or in the organism from which such cell is derived). In some embodiments, the nucleic acids are not naturally occurring, including chimeric combinations of nucleic acids encoding various domains from multiple different cell types.

Among the antigen receptors as per the invention are genetically engineered T cell receptors (TCRs) and components thereof, as well as functional non-TCR antigen receptors, such as chimeric antigen receptors (CAR).

### Chimeric Antigen Receptors (CARs)

In some embodiments, the engineered antigen receptors comprise chimeric antigen receptors (CARs), including activating or stimulatory CARs, costimulatory CARs (see WO2014/055668), and/or inhibitory CARs (iCARs, see Fedorov et al., Sci. Transl. Medicine, 5(215) (December, 2013)).

Chimeric antigen receptors (CARs), (also known as Chimeric immunoreceptors, Chimeric T cell receptors, Artificial T cell receptors) are engineered receptors, which graft an arbitrary specificity onto an immune effector cell (T cell). Typically, these receptors are used to graft the specificity of a monoclonal antibody onto a T cell, with transfer of their coding sequence facilitated by retroviral vectors.

CARs generally include an extracellular antigen (or ligand) binding domain linked to one or more intracellular signaling components, in some embodiments, via linkers and/or transmembrane domain(s). Such molecules typically mimic or approximate a signal through a natural antigen receptor, a signal through such a receptor in combination with a costimulatory receptor, and/or a signal through a costimulatory receptor alone.

In some embodiments, the CAR is constructed with a specificity for a particular antigen (or marker or ligand), such as an antigen expressed in a particular cell type to be targeted by adoptive therapy, such as a cancer marker. Thus, the CAR typically includes in its extracellular portion one or more antigen binding molecules, such as one or more antigen-binding fragment, domain, or portion, or one or more antibody variable domains, and/or antibody molecules.

The moieties used to bind to antigen fall in three general categories, either single-chain antibody fragments (scFvs) derived from antibodies, Fab's selected from libraries, or natural ligands that engage their cognate receptor (for the first generation of CARs). Successful examples in each of these categories are notably reported in Sadelain M, Brentjens R, Riviere I. The basic principles of chimeric antigen receptor (CAR) design. Cancer discovery. 2013; 3(4):388-398 (see notably table 1) and are included in the present application. scFv's derived from murine immunoglobulins are commonly used, as they are easily derived from well-characterized monoclonal antibodies.

Typically, the CAR includes an antigen-binding portion or portions of an antibody molecule, such as a single-chain antibody fragment (scFv) derived from the variable heavy (VH) and variable light (VL) chains of a monoclonal antibody (mAb).

In some embodiments, the CAR comprises an antibody heavy chain domain that specifically binds the antigen, such as a cancer marker or cell surface antigen of a cell or disease to be targeted, such as a tumor cell or a cancer cell, such as any of the target antigens described herein or known in the art.

In some embodiments, the CAR contains an antibody or an antigen-binding fragment (e.g. scFv) that specifically recognizes an antigen, such as an intact antigen, expressed on the surface of a cell.

In some embodiments, the CAR contains a TCR-like antibody, such as an antibody or an antigen-binding fragment (e.g. scFv) that specifically recognizes an intracellular antigen, such as a tumor-associated antigen, presented on the cell surface as a MHC-peptide complex. In some embodiments, an antibody or antigen-binding portion thereof that recognizes an MHC-peptide complex can be expressed on cells as part of a recombinant receptor, such as an antigen receptor. Among the antigen receptors are functional non-TCR antigen receptors, such as chimeric antigen receptors (CARs). Generally, a CAR containing an antibody or antigen-binding fragment that exhibits TCR-like specificity directed against peptide-MHC complexes also may be referred to as a TCR-like CAR.

In some aspects, the antigen-specific binding, or recognition component is linked to one or more transmembrane and intracellular signaling domains. In some embodiments, the CAR includes a transmembrane domain fused to the extracellular domain of the CAR. In one embodiment, the transmembrane domain that is naturally associated with one of the domains in the CAR is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain can be derived from any membrane-bound or transmembrane protein. Transmembrane regions include those derived from (i.e. comprise at least the transmembrane region(s) of) the alpha, beta or zeta chain of the T-cell receptor, CD28, CD3 epsilon, CD45, CD4, CD5, CDS, CD9, CD 16, CD22, CD33, CD37, CD64, CD80, CD86, CD 134, CD137, CD 154). The transmembrane domain can also be synthetic.

In some embodiments, a short oligo- or polypeptide linker, for example, a linker of between 2 and 10 amino acids in length, is present and forms a linkage between the transmembrane domain and the cytoplasmic signaling domain of the CAR.

The CAR generally includes at least one intracellular signaling component or components. First generation CARs typically had the intracellular domain from the CD3 ζ-chain, which is the primary transmitter of signals from endogenous TCRs. Second generation CARs typically further comprise intracellular signaling domains from various costimulatory protein receptors (e.g., CD28, 41BB, ICOS) to the cytoplasmic tail of the CAR to provide additional signals to the T cell. Preclinical studies indicated that the second generation improves the antitumor activity of T cells. More recently, third generation CARs combine multiple signaling domains, such as CD3z-CD28-41BB or CD3z-CD28-OX40, to augment potency.

For example, the CAR can include an intracellular component of the TCR complex, such as a TCR CD3+ chain that mediates T-cell activation and cytotoxicity, e.g., the CD3 zeta chain. Thus, in some aspects, the antigen binding molecule is linked to one or more cell signaling modules. In some embodiments, cell signaling modules include CD3 transmembrane domain, CD3 intracellular signaling domains, and/or other CD transmembrane domains. The CAR can also further include a portion of one or more additional molecules such as Fc receptor γ, CD8, CD4, CD25, or CD16.

In some embodiments, upon ligation of the CAR, the cytoplasmic domain or intracellular signaling domain of the CAR activates at least one of the normal effector functions or responses of the corresponding non-engineered immune cell (typically a T cell). For example, the CAR can induce a function of a T cell such as cytolytic activity or T-helper activity, secretion of cytokines or other factors.

In some embodiments, the intracellular signaling domain(s) include the cytoplasmic sequences of the T cell receptor (TCR), and in some aspects also those of co-receptors that in the natural context act in concert with such receptor to initiate signal transduction following antigen receptor engagement, and/or any derivative or variant of such molecules, and/or any synthetic sequence that has the same functional capability. T cell activation is in some aspects described as being mediated by two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the CAR includes one or both of such signaling components.

In some aspects, the CAR includes a primary cytoplasmic signaling sequence that regulates primary activation of the TCR complex either in a stimulatory way, or in an inhibitory way. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from TCR zeta, FcR gamma, FcR beta, CD3 gamma, CD3 delta, CD3 epsilon, CDS, CD22, CD79a, CD79b, and CD66d. In some embodiments, cytoplasmic signaling molecule(s) in the CAR contain(s) a cytoplasmic signaling domain, portion thereof, or sequence derived from CD3 zeta.

The CAR can also include a signaling domain and/or transmembrane portion of a costimulatory receptor, such as CD28, 4-1BB, OX40, DAP10, and ICOS. In some aspects, the same CAR includes both the activating and costimulatory components; alternatively, the activating domain is provided by one CAR whereas the costimulatory component is provided by another CAR recognizing another antigen.

In some embodiments, the CAR is a CD19 BBz CAR, as typically known in the literature. Typically such CAR comprises the following construct: scFv antiCD19 (FMC63)-CD8 hinge and transmembrane-CD3z intracellular. Optionally the construct comprises a CD8 signal Peptide, as follow: CD8 signal Peptide-scFv antiCD19 (FMC63)-CD8 hinge and transmembrane-CD3z intracellular.

The CAR or other antigen receptor can also be an inhibitory CAR (e.g. iCAR) and includes intracellular components that dampen or suppress a response, such as an immune response. Examples of such intracellular signaling components are those found on immune checkpoint molecules, including PD-1, CTLA4, LAG3, BTLA, OX2R, TIM-3, TIGIT, LAIR-1, PGE2 receptors, EP2/4 Adenosine receptors including A2AR. In some aspects, the engineered cell includes an inhibitory CAR including a signaling domain of or derived from such an inhibitory molecule, such that it serves to dampen the response of the. Such CARs are used, for example, to reduce the likelihood of off-target effects when the antigen recognized by the activating receptor, e.g, CAR, is also expressed, or may also be expressed, on the surface of normal cells.

### TCRs

In some embodiments, the genetically engineered antigen receptors include recombinant T cell receptors (TCRs) and/or TCRs cloned from naturally occurring T cells.

A “T cell receptor” or “TCR” refers to a molecule that contains a variable a and β chains (also known as TCRa and TCRp, respectively) or a variable γ and δ chains (also known as TCRy and TCR5, respectively) and that is capable of specifically binding to an antigen peptide bound to a MHC receptor. In some embodiments, the TCR is in the αβ form. Typically, TCRs that exist in αβ and γδ forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules. In some embodiments, a TCR also can contain a constant domain, a transmembrane domain and/or a short cytoplasmic tail (see, e.g., Janeway et ah, Immunobiology: The Immune System in Health and Disease, 3 rd Ed., Current Biology Publications, p. 4:33, 1997). For example, in some aspects, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction. Unless otherwise stated, the term “TCR” should be understood to encompass functional TCR fragments thereof. The term also encompasses intact or full-length TCRs, including TCRs in the αβ form or γδ form.

Thus, for purposes herein, reference to a TCR includes any TCR or functional fragment, such as an antigen-binding portion of a TCR that binds to a specific antigenic peptide bound in an MHC molecule, i.e. MHC-peptide complex. An “antigen-binding portion” or antigen-binding fragment” of a TCR, which can be used interchangeably, refers to a molecule that contains a portion of the structural domains of a TCR, but that binds the antigen (e.g. MHC-peptide complex) to which the full TCR binds. In some cases, an antigen-binding portion contains the variable domains of a TCR, such as variable a chain and variable β chain of a TCR, sufficient to form a binding site for binding to a specific MHC-peptide complex, such as generally where each chain contains three complementarity determining regions.

In some embodiments, the variable domains of the TCR chains associate to form loops, or complementarity determining regions (CDRs) analogous to immunoglobulins, which confer antigen recognition and determine peptide specificity by forming the binding site of the TCR molecule and determine peptide specificity. Typically, like immunoglobulins, the CDRs are separated by framework regions (FRs) {see, e.g., Jores et al., Pwc. Nat'l Acad. Sci. U.S.A. 87:9138, 1990; Chothia et al., EMBO J. 7:3745, 1988; see also Lefranc et al., Dev. Comp. Immunol. 27:55, 2003). In some embodiments, CDR3 is the main CDR responsible for recognizing processed antigen, although CDR1 of the alpha chain has also been shown to interact with the N-terminal part of the antigenic peptide, whereas CDR1 of the beta chain interacts with the C-terminal part of the peptide. CDR2 is thought to recognize the MHC molecule. In some embodiments, the variable region of the p-chain can contain a further hypervariability (HV4) region.

In some embodiments, the TCR chains contain a constant domain. For example, like immunoglobulins, the extracellular portion of TCR chains {e.g., α-chain, β-chain) can contain two immunoglobulin domains, a variable domain {e.g., Va or Vp; typically amino acids 1 to 116 based on Kabat numbering Kabat et al., “Sequences of Proteins of Immunological Interest, US Dept. Health and Human Services, Public Health Service National Institutes of Health, 1991, 5th ed.) at the N-terminus, and one constant domain {e.g., α-chain constant domain or Ca, typically amino acids 117 to 259 based on Kabat, p-chain constant domain or Cp, typically amino acids 117 to 295 based on Kabat) adjacent to the cell membrane. For example, in some cases, the extracellular portion of the TCR formed by the two chains contains two membrane-proximal constant domains, and two membrane-distal variable domains containing CDRs. The constant domain of the TCR domain contains short connecting sequences in which a cysteine residue forms a disulfide bond, making a link between the two chains. In some embodiments, a TCR may have an additional cysteine residue in each of the a and β chains such that the TCR contains two disulfide bonds in the constant domains.

In some embodiments, the TCR chains can contain a transmembrane domain. In some embodiments, the transmembrane domain is positively charged. In some cases, the TCR chains contain a cytoplasmic tail. In some cases, the structure allows the TCR to associate with other molecules like CD3. For example, a TCR containing constant domains with a transmembrane region can anchor the protein in the cell membrane and associate with invariant subunits of the CD3 signaling apparatus or complex.

Generally, CD3 is a multi-protein complex that can possess three distinct chains (γ, δ, and ε) in mammals and the ζ-chain. For example, in mammals the complex can contain a CD3y chain, a CD35 chain, two CD3s chains, and a homodimer of CD3ζ chains. The CD3y, CD35, and CD3s chains are highly related cell surface proteins of the immunoglobulin superfamily containing a single immunoglobulin domain. The transmembrane regions of the CD3y, CD35, and CD3s chains are negatively charged, which is a characteristic that allows these chains to associate with the positively charged T cell receptor chains. The intracellular tails of the CD3y, CD35, and CD3s chains each contain a single conserved motif known as an immunoreceptor tyrosine-based activation motif or ITAM, whereas each CD3ζ chain has three. Generally, ITAMs are involved in the signaling capacity of the TCR complex. These accessory molecules have negatively charged transmembrane regions and play a role in propagating the signal from the TCR into the cell. The CD3- and ζ-chains, together with the TCR, form what is known as the T cell receptor complex.

In some embodiments, the TCR may be a heterodimer of two chains a and p (or optionally γ and δ) or it may be a single chain TCR construct. In some embodiments, the TCR is a heterodimer containing two separate chains (a and p chains or γ and δ chains) that are linked, such as by a disulfide bond or disulfide bonds.

Recombinant HLA-independent (or non-HLA restricted) T cell receptors (referred to as “HI-TCRs”) that bind to an antigen of interest in an HLA-independent manner are described in International Application No. WO 2019/157454. Such HI-TCRs comprise an antigen binding chain that comprises: (a) an antigen-binding domain that binds to an antigen in an HLA-independent manner, for example, an antigen-binding fragment of an immunoglobulin variable region; and (b) a constant domain that is capable of associating with (and consequently activating) a CD3ζ polypeptide. Because typically TCRs bind antigen in a HLA-dependent manner, the antigen-binding domain that binds in an HLA-independent manner must be heterologous. Preferably, the antigen-binding domain or fragment thereof comprises: (i) a heavy chain variable region (VH) of an antibody and/or (ii) a light chain variable region (VL) of an antibody. The constant domain of the TCR is, for example, a native or modified TRAC polypeptide, or a native or modified TRBC polypeptide. The constant domain of the TCR is, for example, a native TCR constant domain (alpha or beta) or fragment thereof. Unlike chimeric antigen receptors, which typically themselves comprise an intracellular signaling domain, the HI-TCR does not directly produce an activating signal; instead, the antigen-binding chain associates with and consequently activates a CD3ζ polypeptide. The immune cells comprising the recombinant TCR provide superior activity when the antigen has a low density on the cell surface of less than about 10,000 molecules per cell.

The CD3ζ polypeptide is, for example, a native CD3ζ polypeptide or a modified CD3ζ polypeptide. The CD3ζ polypeptide is optionally fused to an intracellular domain of a co-stimulatory molecule or a fragment thereof. Alternatively, the antigen binding domain optionally comprises a co-stimulatory region, e.g. intracellular domain, that is capable of stimulating an immunoresponsive cell upon the binding of the antigen binding chain to the antigen. Example co-stimulatory molecules include CD28, 4-1 BB, OX40, ICOS, DAP-10, fragments thereof, or a combination thereof. In some embodiments, the recombinant HI-TCR is expressed by a transgene that is integrated at an endogenous gene locus of the immunoresponsive cell, for example, a CD3δ locus, a CD3ε locus, a CD247 locus, a B2M locus, a TRAC locus, a TRBC locus, a TRDC locus and/or a TRGC locus. In most embodiments, expression of the recombinant HI-TCR is driven from the endogenous TRAC or TRBC gene locus. In some embodiments, the transgene encoding a portion of the recombinant HI-TCR is integrated into the endogenous TRAC and/or TRBC locus in a manner that disrupts or abolishes the endogenous expression of a TCR comprising a native TCR α chain and/or a native TCR β chain. This disruption prevents or eliminates mispairing between the recombinant TCR and a native TCR α chain and/or a native TCR β chain in the immunoresponsive cell. The endogenous gene locus may also comprise a modified transcription terminator region, for example, a TK transcription terminator, a GCSF transcription terminator, a TCRA transcription terminator, an HBB transcription terminator, a bovine growth hormone transcription terminator, an SV40 transcription terminator, and a β2A element.

The recombinant HI-TCR may be further combined with other features in a immune cell of the present invention. For example, the immune cell is a cell wherein the antigen-specific receptor is a modified TCR comprising a heterologous antigen-binding domain and a native TCR constant domain or fragment thereof, and the antigen-specific receptor is capable of activating a CD3 zeta polypeptide. For example, the immune cell may further comprise at least one chimeric costimulatory receptor (CCR) and/or at least one chimeric antigen receptor.

Furthermore, in the immune cells, the nucleic acid encoding the antigen-binding domain of the HI-TCR may be inserted into the endogenous TRAC locus and/or TRBC locus of the immune cell. The insertion of the HI-TCR nucleic acid sequence, or another smaller mutation, can disrupt or abolish the endogenous expression of a TCR comprising a native TCR alpha chain and/or a native TCR beta chain. The insertion or mutation may reduce endogenous TCR expression by at least about 50%, 60%, 70%, 75%, 80%, 85%, 90%, 95%, 98%, or 99%. Because a single gene encodes the alpha chain (TRAC) rather than the two genes encoding the beta chain, the TRAC locus is a typical target for reducing TCRαβ receptor expression. Thus, the nucleic acid encoding the antigen-specific receptor (e.g. CAR or TCR) may be integrated into the TRAC locus at a location, preferably in the 5′ region of the first exon, that significantly reduces expression of a functional TCR alpha chain. See, e.g., Jantz et al., WO 2017/062451; Sadelain et al., WO 2017/180989; Torikai et al., Blood, 119(2): 5697-705 (2012); Eyquem et al., Nature. 2017 Mar. 2; 543(7643):113-117. Expression of the endogenous TCR alpha may be reduced by at least about 50%, 60%, 70%, 75%, 80%, 85%, 90%, 95%, 98%, or 99%. In such embodiments, expression of the nucleic acid encoding the antigen-specific receptor is optionally under control of the endogenous TCR-alpha or endogenous TCR-beta promoter.

Optionally, the immune cell may also comprise a modified CD3 with a single active ITAM domain, and optionally the CD3 may further comprise one or more or two or more costimulatory domains. In some embodiments, the CD3 comprises two costimulatory domains, optionally CD28 and 4-1BB. The modified CD3 with a single active ITAM domain can comprise, for example, a modified CD3zeta intracellular signaling domain in which ITAM2 and ITAM3 have been inactivated, or ITAM1 and ITAM2 have been inactivated. In some embodiments, a modified CD3 zeta polypeptide retains only ITAM1 and the remaining CD3ζ domain is deleted (residues 90-164). As another example, ITAM1 is substituted with the amino acid sequence of ITAM3, and the remaining CD3ζ domain is deleted (residues 90-164).

The modified immune cells of the present invention may thus further comprise combinations of two or more, or three or more, or four or more, of the foregoing aspects.

For example, the modified immune cell is an immune cell wherein (a) the antigen-specific receptor is a modified TCR comprising a heterologous antigen-binding domain and a native TCR constant domain or fragment thereof, and the antigen-specific receptor is capable of activating a CD3 zeta polypeptide, and/or the antigen-specific receptor is a CAR, and optionally (b) the immune cell comprises a modified CD3 with a single active ITAM domain, e.g. in which ITAM2 and ITAM3 have been inactivated, and optionally (c) the TCR is under control of an endogenous TRAC and/or TRBC promoter, and optionally (d) expression of native TCR-alpha chain and/or native TCR-beta chain are disrupted or abolished. In further embodiments, the cell may comprise at least one chimeric costimulatory receptor (CCR).

Exemplary antigen receptors, including CARs and recombinant TCRs, as well as methods for engineering and introducing the receptors into cells, include those described, for example, in international patent application publication numbers WO200014257, WO2013126726, WO2012/129514, WO2014031687, WO2013/166321, WO2013/071154, WO2013/123061 U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., Cancer Discov. 2013 April; 3(4): 388-398; Davila et al. (2013) PLoS ONE 8(4): e61338; Turtle et al., Curr. Opin. Immunol., 2012 October; 24(5): 633-39; Wu et al., Cancer, 2012 March 18(2): 160-75. In some aspects, the genetically engineered antigen receptors include a CAR as described in U.S. Pat. No. 7,446,190, and those described in International Patent Application Publication No.: WO/2014055668 A1.

### Antigens

Among the antigens targeted by the genetically engineered antigen receptors are those expressed in the context of a disease, condition, or cell type to be targeted via the adoptive cell therapy. Among the diseases and conditions are proliferative, neoplastic, and malignant diseases and disorders, more particularly cancers, thus in some embodiment the one or more antigens are selected from tumor antigen (e.g. expressed by tumor cells, notably specifically expressed by cancer cells).

The cancer may be a solid cancer or a “liquid tumor” such as cancers affecting the blood, bone marrow and lymphoid system, also known as tumors of the hematopoietic and lymphoid tissues, which notably include leukemia and lymphoma. Liquid tumors include for example acute myelogenous leukemia (AML), chronic myelogenous leukemia (CML), acute lymphocytic leukemia (ALL), and chronic lymphocytic leukemia (CLL), (including various lymphomas such as mantle cell lymphoma, non-Hodgkins lymphoma (NHL), adenoma, squamous cell carcinoma, laryngeal carcinoma, gallbladder and bile duct cancers, cancers of the retina such as retinoblastoma).

Solid cancers notably include cancers affecting one of the organs selected from the group consisting of colon, rectum, skin, endometrium, lung (including non-small cell lung carcinoma), uterus, bones (such as Osteosarcoma, Chondrosarcomas, Ewing's sarcoma, Fibrosarcomas, Giant cell tumors, Adamantinomas, and Chordomas), liver, kidney, esophagus, stomach, bladder, pancreas, cervix, brain (such as Meningiomas, Glioblastomas, Lower-Grade Astrocytomas, Oligodendrocytomas, Pituitary Tumors, Schwannomas, and Metastatic brain cancers), ovary, breast, head and neck region, testis, prostate and the thyroid gland.

Preferably, a cancer according to the invention is a cancer affecting the blood, bone marrow and lymphoid system as described above. Typically, the cancer is, or is associated, with multiple myeloma.

Diseases according to the invention also encompass infectious diseases or conditions, such as, but not limited to, viral, retroviral, bacterial, and protozoal infections, immunodeficiency, Cytomegalovirus (CMV), Epstein-Barr virus (EBV), adenovirus, BK polyomavirus; autoimmune or inflammatory diseases or conditions, such as arthritis, e.g., rheumatoid arthritis (RA), Type I diabetes, systemic lupus erythematosus (SLE), inflammatory bowel disease, psoriasis, scleroderma, autoimmune thyroid disease, Grave's disease, Crohn's disease multiple sclerosis, asthma, and/or diseases or conditions associated with transplant.

In some embodiments, the antigen is a polypeptide. In some embodiments, it is a carbohydrate or other molecule. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells. In some such embodiments, a multi-targeting and/or gene disruption approach as provided herein is used to improve specificity and/or efficacy.

In some embodiments, the antigen is expressed in a cancer cell and/or is a universal tumor antigen. The term “universal tumor antigen” refers to an immunogenic molecule, such as a protein, that is, generally, expressed at a higher level in tumor cells than in non-tumor cells and also that is expressed in tumors of different origins. In some embodiments, the universal tumor antigen is expressed in more than 30%, 40%, 50%, 60%, 70%, 75%, 80%, 85%, 90% or more of human cancers. In some embodiments, the universal tumor antigen is expressed in at least three, at least four, at least five, at least six, at least seven, at least eight or more different types of tumors. In some cases, the universal tumor antigen may be expressed in non-tumor cells, such as normal cells, but at lower levels than it is expressed in tumor cells. In some cases, the universal tumor antigen is not expressed at all in non-tumor cells, such as not expressed in normal cells. Exemplary universal tumor antigens include, for example, human telomerase reverse transcriptase (hTERT), survivin, mouse double minute 2 homolog (MDM2), cytochrome P450 1 B1 (CYP1 B), HER2/neu, Wilms' tumor gene 1 (WT1), livin, alphafetoprotein (AFP), carcinoembryonic antigen (CEA), mucin 16 (MUC16), MUC1, prostate-specific membrane antigen (PSMA), p53 or cyclin (DI). Peptide epitopes of tumor antigens, including universal tumor antigens, are known in the art and, in some aspects, can be used to generate MHC-restricted antigen receptors, such as TCRs or TCR-like CARs (see e.g. published PCT application No. WO2011009173 or WO2012135854 and published U.S. application No. US20140065708).

In some aspects, the antigen is expressed on multiple myeloma, such as CD38, CD138, and/or CS-1. Other exemplary multiple myeloma antigens include CD56, TIM-3, CD33, CD 123, and/or CD44. Antibodies or antigen-binding fragments directed against such antigens are known and include, for example, those described in U.S. Pat. Nos. 8,153,765; 8,603,477, 8,008,450; U.S. published application No. US20120189622; and published international PCT application Nos. WO2006099875, WO2009080829 or WO2012092612. In some embodiments, such antibodies or antigen-binding fragments thereof (e.g. scFv) can be used to generate a CAR.

In some embodiments, the antigen may be one that is expressed or upregulated on cancer or tumor cells, but that also may be expressed in an immune cell, such as a resting or activated T cell. For example, in some cases, expression of hTERT, survivin and other universal tumor antigens are reported to be present in lymphocytes, including activated T lymphocytes (see e.g., Weng et al. (1996) J Exp. Med., 183:2471-2479; Hathcock et al. (1998) J Immunol, 160:5702-5706; Liu et al. (1999) Proc. Natl Acad Sci., 96:5147-5152; Turksma et al. (2013) Journal of Translational Medicine, 11: 152). Likewise, in some cases, CD38 and other tumor antigens also can be expressed in immune cells, such as T cells, such as upregulated in activated T cells. For example, in some aspects, CD38 is a known T cell activation marker.

In some embodiments as provided herein, an immune cell, such as a T cell, can be engineered to repress or disrupt the gene encoding the antigen in the immune cell so that the expressed genetically engineered antigen receptor does not specifically bind the antigen in the context of its expression on the immune cell itself. Thus, in some aspects, this may avoid off-target effects, such as binding of the engineered immune cells to themselves, which may reduce the efficacy of the engineered in the immune cells, for example, in connection with adoptive cell therapy.

In some embodiments, such as in the case of an inhibitory CAR, the target is an off-target marker, such as an antigen not expressed on the diseased cell or cell to be targeted, but that is expressed on a normal or non-diseased cell which also expresses a disease-specific target being targeted by an activating or stimulatory receptor in the same engineered cell. Exemplary such antigens are MHC molecules, such as MHC class I molecules, for example, in connection with treating diseases or conditions in which such molecules become downregulated but remain expressed in non-targeted cells.

In some embodiments, the engineered immune cells can contain an antigen that targets one or more other antigens. In some embodiments, the one or more other antigens is a tumor antigen or cancer marker. Other antigen targeted by antigen receptors on the provided immune cells can, in some embodiments, include orphan tyrosine kinase receptor ROR1, tEGFR, Her2, LI-CAM, CD19, CD20, CD22, mesothelin, CEA, and hepatitis B surface antigen, anti-folate receptor, CD23, CD24, CD30, CD33, CD38, CD44, EGFR, EGP-2, EGP-4, EPHa2, ErbB2, 3, or 4, FBP, fetal acethycholine e receptor, GD2, GD3, HMW-MAA, IL-22R-alpha, IL-13R-alpha2, kdr, kappa light chain, Lewis Y, LI-cell adhesion molecule, MAGE-A1, mesothelin, MUC1, MUC16, PSCA, NKG2D Ligands, NY-ESO-1, MART-1, gplOO, oncofetal antigen, ROR1, TAG72, VEGF-R2, carcinoembryonic antigen (CEA), prostate specific antigen, PSMA, Her2/neu, estrogen receptor, progesterone receptor, ephrinB2, CD 123, CS-1, c-Met, GD-2, and MAGE A3, CE7, Wilms Tumor 1 (WT-1), a cyclin, such as cyclin Al (CCNA1), and/or biotinylated molecules, and/or molecules expressed by HIV, HCV, HBV or other pathogens.

For example, the one or more antigens can be selected from tumor antigens from the group comprising pHER95, CD19, MUC16, MUC1, CAIX, CEA, CD8, CD7, CD10, CD20, CD22, CD30, CD70, CLL1, CD33, CD34, CD38, CD41, CD44, CD49f, CD56, CD74, CD133, CD138, EGP-2, EGP-40, EpCAM, Erb-B2, Erb-B3, Erb-B4, FBP, Fetal acetylcholine receptor, folate receptor-a, GD2, GD3, HER-2, hTERT, IL-13R-a2, κ-light chain, KDR, LeY, L1 cell adhesion molecule, MAGE-A1, Mesothelin, MAGEA3, p53, MART1, GP100, Proteinase3 (PR1), Tyrosinase, Survivin, hTERT, EphA2, NKG2D ligands, NY-ESO-1, oncofetal antigen (h5T4), PSCA, PSMA, ROR1, TAG-72, VEGF-R2, WT-1, BCMA, CD123, CD44V6, NKCS1, EGF1R, EGFR-VIII, CD99, CD70, ADGRE2, CCR1, LILRB2, LILRB4, PRAME, and ERBB.

In some embodiments, the CAR binds a pathogen-specific antigen. In some embodiments, the CAR is specific for viral antigens (such as HIV, HCV, HBV, etc.), bacterial antigens, and/or parasitic antigens.

In some embodiments, the CAR includes encompasses one or more 4-1 BB co-stimulatory domain and binds a CD19 antigen (also known as 19BBz CAR in the literature).

In some embodiments, the cells of the invention are genetically engineered to express two or more genetically engineered receptors on the cell, each recognizing a different antigen and typically each including a different intracellular signaling component. Such multi-targeting strategies are described, for example, in International Patent Application, Publication No.: WO 2014055668 A1 (describing combinations of activating and costimulatory CARs, e.g., targeting two different antigens present individually on off-target, e.g., normal cells, but present together only on cells of the disease or condition to be treated) and Fedorov et al., Sci. Transl. Medicine, 5(215) (December, 2013) (describing cells expressing an activating and an inhibitory CAR, such as those in which the activating CAR binds to one antigen expressed on both normal or non-diseased cells and cells of the disease or condition to be treated, and the inhibitory CAR binds to another antigen expressed only on the normal cells or cells which it is not desired to treat).

In some contexts, overexpression of a stimulatory factor (for example, a lymphokine or a cytokine) may be toxic to a subject. Thus, in some contexts, the engineered cells include gene segments that cause the cells to be susceptible to negative selection in vivo, such as upon administration in adoptive immunotherapy. For example in some aspects, the cells are engineered so that they can be eliminated as a result of a change in the in vivo condition of the patient to which they are administered. The negative selectable phenotype may result from the insertion of a gene that confers sensitivity to an administered agent, for example, a compound. Negative selectable genes include the Herpes simplex virus type I thymidine kinase (HSV-I TK) gene (Wigler et al., Cell II: 223, 1977) which confers ganciclovir sensitivity; the cellular hypoxanthine phosphribosyltransferase (HPRT)gene, the cellular adenine phosphoribosyltransferase (APRT) gene, bacterial cytosine deaminase, (Mullen et al., Proc. Natl. Acad. Sci. USA. 89:33 (1992)).

In other embodiments of the invention, the cells, e.g., T cells, are not engineered to express recombinant receptors, but rather include naturally occurring antigen receptors specific for desired antigens, such as tumor-infiltrating lymphocytes and/or T cells cultured in vitro or ex vivo, e.g., during the incubation step(s), to promote expansion of cells having particular antigen specificity. For example, in some embodiments, the cells are produced for adoptive cell therapy by isolation of tumor-specific T cells, e.g. autologous tumor infiltrating lymphocytes (TIL). The direct targeting of human tumors using autologous tumor infiltrating lymphocytes can in some cases mediate tumor regression (see Rosenberg S A, et al. (1988) N Engl J Med. 319: 1676-1680). In some embodiments, lymphocytes are extracted from resected tumors. In some embodiments, such lymphocytes are expanded in vitro. In some embodiments, such lymphocytes are cultured with lymphokines (e.g., IL-2). In some embodiments, such lymphocytes mediate specific lysis of autologous tumor cells but not allogeneic tumor or autologous normal cells.

Among additional nucleic acids, e.g., genes for introduction are those to improve the efficacy of therapy, such as by promoting viability and/or function of transferred cells; genes to provide a genetic marker for selection and/or evaluation of the cells, such as to assess in vivo survival or localization; genes to improve safety, for example, by making the cell susceptible to negative selection in vivo as described by Lupton S. D. et al., Mol. and Cell Biol., 11:6 (1991); and Riddell et al., Human Gene Therapy 3:319-338 (1992); see also the publications of PCT/US91/08442 and PCT/US94/05601 by Lupton et al. describing the use of bifunctional selectable fusion genes derived from fusing a dominant positive selectable marker with a negative selectable marker. See, e.g., Riddell et al., U.S. Pat. No. 6,040,177, at columns 14-17.

### Method for Obtaining Cells According to the Invention

The present invention also relates to a method of producing a modified or engineered immune cell, comprising a step consisting in inhibiting of the expression and/or activity of SOCS1 and/or FAS and/or Suv39h1 in the immune cell.

Preferably, the method for obtaining cells according to the invention further comprises a step consisting in introducing into said immune cells of a genetically engineered antigen receptor that specifically binds to a target antigen, or a T cell receptor.

The inhibition of the expression and/or activity of SOCS1 (and in some embodiments the additional inhibition of the expression and/or activity of FAS and/or Suv39h1) and the introduction of a genetically engineered antigen receptor that specifically binds to a target antigen in the immune cell can be carried out simultaneously or sequentially in any order.

Inhibition of SOCS1, FAS Suv39h1 and/or β32m

The methods as herein described for inhibition of the gene expression or of the activity of the protein apply to the 4 genes/proteins of interest, namely SOCS1, FAS, Suv39h1 and optionally β2m. When the cell is defective for more than SOCS1, the same of different method(s) can be used to render the cell further defective for FAS and/or Suv39h1. Embodiments as described herein can therefore be combined according to the skilled person knowledge.

According to the invention, the engineered immune cell can be contacted with at least one agent that inhibits or blocks the expression and/or activity of SOCS1 and optionally in some embodiments with at least one additional agent that inhibits or blocks the expression and/or activity of Suv39h1, FAS and/or β2m. The present invention also provides embodiments wherein Fas is inactivated in the immune cell (notably cells), optionally in combination with Suv39 and/or 32m.

Said agent can be selected from small molecule inhibitors; peptide inhibitors, antibodies derivatives such as intrabodies, nanobodies or affibodies; aptamers; nucleic acid molecules that block transcription or translation, such as antisense molecules complementary to SOCS1, FAS or Suv39h1; RNA interfering agents (such as a small interfering RNA (siRNA), small hairpin RNA (shRNA), microRNA (miRNA), or a piwiRNA (piRNA); ribozymes and combination thereof.

The at least one agent can also be an exogenous nucleic acid comprising a) one or more engineered, non-naturally occurring Clustered Regularly Interspaced Short Palindromic Repeats (CRISPR) guide RNA that hybridizes with SOCS1, Suv39h1, FAS or 32_n genomic nucleic acid sequence and/or b) a nucleotide sequence encoding a CRISPR protein (typically a Type-II Cas9 protein), optionally wherein the cells are transgenic for expressing a Cas9 protein. The agent may also be a Zinc finger protein (ZFN) or a TAL protein.

The term “small organic molecule” refers to a molecule of a size comparable to those organic molecules generally used in pharmaceuticals. The term excludes biological macro molecules (e. g., proteins, nucleic acids, etc.). Preferred small organic molecules range in size up to about 5000 Da, more preferably up to 2000 Da, and most preferably up to about 1000 Da.

In some embodiments, an inhibitor of H3K9-histone methyltransferase SUV39H1 is chaetocin (CAS 28097-03-2), as described by Greiner D, Bonaldi T, Eskeland R, Roemer E, Imhof A. “Identification of a specific inhibitor of the histone methyltransferase SU(VAR)3-9”. Nat Chem Biol. 2005 August; I(3): 143-5; Weber, H. P., et al, “The molecular structure and absolute configuration of chaetocin”, Acta Cryst, B28, 2945-2951 (1972); Udagawa, S., et al, “The production of chaetoglobosins, sterigmatocystin, O-methylsterigmatocystin, and chaetocin by Chaetomium spp. and related fungi”, Can. J. microbiol, 25, 170-177 (1979); and Gardiner, D. M., et al, “The epipolythiodioxopiperazine (ETP) class of fungal toxins: distribution, mode of action, functions and biosynthesis”, Microbiol, 151, 1021-1032 (2005). For example, chaetocin is commercially available from Sigma Aldrich.

Another inhibitor of Suv39h1 can also be ETP69 (Rac-(3S,6S,7S,8aS)-6-(benzo[d][1,3]dioxol-5-yl)-2,3,7-trimethyl-1,4-dioxohexahydro-6H-3,8a-epidithiopyrrolo[1,2-a]pyrazine-7-carbonitrile), a racemic analog of the epidithiodiketopiperazine alkaloid chaetocin A (see WO2014066435 but see also Baumann M, Dieskau A P, Loertscher B M, et al. Tricyclic Analogues of Epidithiodioxopiperazine Alkaloids with Promising In Vitro and In Vivo Antitumor Activity. Chemical science (Royal Society of Chemistry: 2010). 2015; 6:4451-4457, and Snigdha S, Prieto G A, Petrosyan A, et al. H3K9me3 Inhibition Improves Memory, Promotes Spine Formation, and Increases BDNF Levels in the Aged Hippocampus. The Journal of Neuroscience. 2016; 36(12):3611-3622).

The inhibiting activity of a compound may be determined using various methods as described in Greiner D. Et al. Nat Chem Biol. 2005 August; I(3): 143-5 or Eskeland, R. et al. Biochemistry 43, 3740-3749 (2004).

Inhibition of SOCS1, FAS, Suv39h1 and/or 32m in the cell can be achieved before or after injection in the targeted patient. In some embodiment, inhibition as previously defined is performed in vivo after administration of the cell to the subject. For example, a Suv39h1 inhibitor as herein defined can be included in the composition containing the cell. One or more SOCS1, FAS, Suv39h1 or 32m inhibitor(s) may also be administered separately before, concomitantly of after administration of the cell(s) to the subject.

Typically, inhibition of SOCS1, FAS, Suv39h1 and/or 32 according to the present application may be achieved with incubation of a cell according to the invention with a composition containing at least one pharmacological inhibitor as previously described. The inhibitor is included during the expansion of the anti-tumor T cells in vitro, thus modifying their reconstitution, survival and therapeutic efficacy after adoptive transfer.

Inhibition of SOCS1, FAS, Suv39h1 and/or 32 in a cell according to the invention may be achieved with intrabodies. Intrabodies are antibodies that bind intracellularly to their antigen after being produced in the same cell (for a review se for example, Marschall A L, Dubel S and Böldicke T “Specific in vivo knockdown of protein function by intrabodies”, MAbs. 2015; 7(6):1010-35. but see also Van Impe K, Bethuyne J, Cool S, Impens F, Ruano-Gallego D, De Wever O, Vanloo B, Van Troys M, Lambein K, Boucherie C, et al. “A nanobody targeting the F-actin capping protein CapG restrains breast cancer metastasis”. Breast Cancer Res 2013; 15:R116; Hyland S, Beerli R R, Barbas C F, Hynes N E, Wels W. “Generation and functional characterization of intracellular antibodies interacting with the kinase domain of human EGF receptor. Oncogene 2003; 22:1557-67”; Lobato M N, Rabbitts T H. “Intracellular antibodies and challenges facing their use as therapeutic agents”. Trends Mol Med 2003; 9:390-6, and Donini M, Morea V, Desiderio A, Pashkoulov D, Villani M E, Tramontano A, Benvenuto E. “Engineering stable cytoplasmic intrabodies with designed specificity”. J Mol Biol. 2003 Jul. 4; 330(2):323-32).

Intrabodies can be generated by cloning the respective cDNA from an existing hybridoma clone or more conveniently, new scFvs/Fabs can be selected from in vitro display techniques such as phage display which provide the necessary gene encoding the antibody from the onset and allow a more detailed predesign of antibody fine specificity. In addition, bacterial-, yeast-, mammalian cell surface display and ribosome display can be employed. However, the most commonly used in vitro display system for selection of specific antibodies is phage display. In a procedure called panning (affinity selection), recombinant antibody phages are selected by incubation of the antibody phage repertoire with the antigen. This process is repeated several times leading to enriched antibody repertoires comprising specific antigen binders to almost any possible target. To date, in vitro assembled recombinant human antibody libraries have already yielded thousands of novel recombinant antibody fragments. It is to be noted that the prerequisite for a specific protein knockdown by a cytoplasmic intrabody is that the antigen is neutralized/inactivated through the antibody binding. Five different approaches to generate suitable antibodies have emerged: 1) In vivo selection of functional intrabodies in eukaryotes such as yeast and in prokaryotes such as E. coli (antigen-dependent and independent); 2) generation of antibody fusion proteins for improving cytosolic stability; 3) use of special frameworks for improving cytosolic stability (e.g., by grafting CDRs or introduction of synthetic CDRs in stable antibody frameworks); 4) use of single domain antibodies for improved cytosolic stability; and 5) selection of disulfide bond free stable intrabodies. Those approaches are notably detailed in Marschall, A. L et al., mAbs 2015 as mentioned above.

The most commonly used format for intrabodies is the scFv, which consists of the H- and L-chain variable antibody domain (VH and VL) held together by a short, flexible linker sequence (frequently (Gly4Ser)3), to avoid the need for separate expression and assembly of the 2 antibody chains of a full IgG or Fab molecule. Alternatively, the Fab format comprising additionally the C1 domain of the heavy chain and the constant region of the light chain has been used. Recently, a new possible format for intrabodies, the scFab, has been described. The scFab format promises easier subcloning of available Fab genes into the intracellular expression vector, but it remains to be seen whether this provides any advantage over the well-established scFv format. In addition to scFv and Fab, bispecific formats have been used as intrabodies. A bispecific Tie-2×VEGFR-2 antibody targeted to the ER demonstrated an extended half-life compared to the monospecific antibody counterparts. A bispecific transmembrane intrabody has been developed as a special format to simultaneously recognize intra- and extracellular epitopes of the epidermal growth factor, combining the distinct features of the related monospecific antibodies, i.e., inhibition of autophosphorylation and ligand binding.

Another intrabody format particularly suitable for cytoplasmic expression are single domain antibodies (also called nanobodies) derived from camels or consisting of one human VH domain or human VL domain. These single domain antibodies often have advantageous properties, e.g., high stability; good solubility; ease of library cloning and selection; high expression yield in E. coli and yeast.

The intrabody gene can be expressed inside the target cell after transfection with an expression plasmid or viral transduction with a recombinant virus. Typically, the choice is aimed at providing optimal intrabody transfection and production levels. Successful transfection and subsequent intrabody production can be analyzed by immunoblot detection of the produced antibody, but, for the evaluation of correct intrabody/antigen-interaction, co-immunoprecipitation from HEK 293 cell extracts transiently cotransfected with the corresponding antigen and intrabody expression plasmids may be used.

Inhibition of SOCS1 and/or FAS and/or Suv39h1 in a cell according to the invention may also be effected with aptamers that inhibit or block SOCS1, FAS or Suv39h1 expression or activity respectively. Aptamers are a class of molecule that represents an alternative to antibodies in term of molecular recognition. Aptamers are oligonucleotide (DNA or RNA) or oligopeptide sequences with the capacity to recognize virtually any class of target molecules with high affinity and specificity.

Oligonucleotide aptamers may be isolated through Systematic Evolution of Ligands by Exponential enrichment (SELEX) of a random sequence library, as described in Tuerk C. and Gold L., 1990. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S. D., 1999.

Peptide aptamers consists of a conformationally constrained antibody variable region displayed by a platform protein, such as E. coli Thioredoxin A that are selected from combinatorial libraries by two hybrid methods (Colas P, Cohen B, Jessen T, Grishina I, McCoy J, Brent R. “Genetic selection of peptide aptamers that recognize and inhibit cyclin-dependent kinase 2”. Nature. 1996 Apr. 11; 380(6574):548-50).

Inhibition of SOCS1, Fas, Suv39h1 and/β2m in a cell according to the invention may also be effected with affibody molecules. Affibody are small proteins engineered to bind to a large number of target proteins or peptides with high affinity, imitating monoclonal antibodies, and are therefore a member of the family of antibody mimetics (see for review Löfblom J, Feldwisch J, Tolmachev V, Carlsson J, Stahl S, Frejd F Y. Affibody molecules: engineered proteins for therapeutic, diagnostic and biotechnological applications. FEBS Lett. 2010 Jun. 18; 584(12):2670-80). Affibody molecules are based on an engineered variant (the Z domain) of the B-domain in the immunoglobulin-binding regions of staphylococcal protein A, with specific binding for theoretically any given target. Affibody molecule libraries are generally constructed by combinatorial randomization of 13 amino acid positions in helices one and two that comprise the original Fc-binding surface of the Z-domain. The libraries have typically been displayed on phages, followed by biopanning against desired targets. Should the affinity of the primary be increased, affinity maturation generally results in improved binders and may be achieved by either helix shuffling or sequence alignment combined with directed combinatorial mutagenesis. The newly identified molecules with their altered binding surface generally keep the original helical structure as well as the high stability, although unique exceptions with interesting properties have been reported. Due to their small size and rapid folding properties, affibody molecules can be produced by chemical peptide synthesis.

In other embodiments of the invention, inhibition of SOCS1 and/or of FAS and/or of Suv39h1 and/or β2m activity can be achieved by gene repression/suppression via gene knockdown using an RNA or DNA, notably a recombinant DNA or RNA, typically using RNA interference (RNAi) such as dsRNA (double-stranded RNA), miRNA (microRNA), short interfering RNA (siRNA) short hairpin RNA (shRNA)n anti-sens RNA or DNA or sequences encoding ribozymes. For the purposes of the invention, the term “recombinant DNA or RNA” refers to a nucleic acid sequence that has been altered, rearranged, or modified by genetic engineering. The term “recombinant” does not refer to alterations of nucleic acid sequences that result from naturally occurring events, such as spontaneous mutations, or from non-spontaneous mutagenesis followed by selective breeding.

As used herein, the term “RNA” refers to a molecule comprising at least one ribonucleotide residue. By “ribonucleotide” is meant a nucleotide with a hydroxyl group at the 2′ position of a .beta.-D-ribofuranose moiety. The terms encompass double stranded RNA, single stranded RNA, RNAs with both double stranded and single stranded regions, isolated RNA such as partially purified RNA, essentially pure RNA, synthetic RNA, recombinantly produced RNA, as well as altered RNA, or analog RNA, that differs from naturally occurring RNA by the addition, deletion, substitution, and/or alteration of one or more nucleotides. Such alterations can include addition of non-nucleotide material, such as to the end(s) of an RNA molecule or internally, for example at one or more nucleotides of the RNA. Nucleotides in the RNA molecules of the presently disclosed subject matter can also comprise non-standard nucleotides, such as non-naturally occurring nucleotides or chemically synthesized nucleotides or deoxynucleotides. These altered RNAs can be referred to as analogs or analogs of a naturally occurring RNA. siRNA technology includes that based on RNAi utilizing a double-stranded RNA molecule having a sequence homologous with the nucleotide sequence of mRNA which is transcribed from the gene, and a sequence complementary with the nucleotide sequence. siRNA generally is homologous/complementary with one region of mRNA which is transcribed from the gene, or may be siRNA including a plurality of RNA molecules which are homologous/complementary with different regions.

Anti-sense oligonucleotides, including anti-sense RNA molecules and anti-sense DNA molecules, would act to directly block the translation of SOCS1, FAS, H3K9-histone methyltransferase Suv39h1, or β2m and thus prevent protein translation or increase mRNA degradation, thus decreasing the level of SOCS1, FAS, H3K9-histone methyltransferase SUV39H1, or β2m respectively and thus its/their activity in a cell. For example, antisense oligonucleotides of at least about 15 bases and complementary to unique regions of the mRNA transcript sequence encoding SOCS1, FAS, H3K9-histone methyltransferase SUV39H1, or β2m can be synthesized, e.g., by conventional phosphodiester techniques and administered by e.g., intravenous injection or infusion. Methods for using antisense techniques for specifically inhibiting gene expression of genes whose sequence is known are well known in the art (see for example U.S. Pat. Nos. 6,566,135; 6,566, 131; 6,365,354; 6,410,323; 6,107,091; 6,046,321; and 5,981,732).

An “RNA interfering agent” as used herein, is defined as any agent, which interferes with or inhibits expression of a target biomarker gene by RNA interference (RNAi). Such RNA interfering agents include, but are not limited to, nucleic acid molecules including RNA molecules, which are homologous to the target gene of the invention (e.g., Suv39h1), or a fragment thereof, short interfering RNA (siRNA), and small molecules which interfere with or inhibit expression of the target nucleic acid by RNA interference (RNAi).

Small inhibitory RNAs (siRNAs) can also function as inhibitors of expression for use according to the present application. SOCS1 gene expression, FAS expression, H3K9-histone methyltransferase SUV39H1, and/or B2M gene expression can be reduced by contacting a subject or cell with a small double stranded RNA (dsRNA), or a vector or construct causing the production of a small double stranded RNA, such that SOCS1 gene expression, FAS expression, H3K9-histone methyltransferase SUV39H1, or B2M gene expression is specifically inhibited (i.e. RNA interference or RNAi). Methods for selecting an appropriate dsRNA or dsRNA-encoding vector are well known in the art for genes whose sequence is known (see for example Tuschl, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836). All or part of the phosphodiester bonds of the siRNAs of the invention are advantageously protected. This protection is generally implemented via the chemical route using methods that are known by art. The phosphodiester bonds can be protected, for example, by a thiol or amine functional group or by a phenyl group. The 5′- and/or 3′-ends of the siRNAs of the invention are also advantageously protected, for example, using the technique described above for protecting the phosphodiester bonds. The siRNAs sequences advantageously comprise at least twelve contiguous dinucleotides or their derivatives.

shRNAs (short hairpin RNA) can also function as inhibitors of expression for use in the present invention. shRNAs are typically composed of a short (e.g., 19-25 nucleotide) antisense strand, followed by a 5-9 nucleotide loop, and the analogous sense strand. Alternatively, the sense strand may precede the nucleotide loop structure and the antisense strand may follow.

As used herein, the term “microRNAs” (miRNA or RNA) refers to single-stranded RNA molecules of 21 to 23 nucleotides in length, preferably 21 to 22 nucleotides, which are capable of regulating gene expression. The miRNAs are each processed from a longer precursor RNA molecule (“precursor miRNA”). Precursor miRNAs are transcribed from non-protein-encoding genes. The precursor miRNAs have two regions of complementarity that enable them to form a stem-loop- or fold-back-like structure. The processed miRNA (also referred to as “mature miRNA”) becomes part of a large complex to down-regulate a particular target gene.

In some embodiments, a recombinant DNA as herein described is a recombinant DNA encoding a ribozyme. Ribozymes can also function as inhibitors of expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleo lytic cleavage. Engineered hairpin or hammerhead motif ribozyme molecules that specifically and efficiently catalyze endonucleolytic cleavage of H3K9-histone methyltransferase SUV39H1 mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable.

Both antisense oligonucleotides and ribozymes useful as inhibitors of expression can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-0-methyl rather than phosphodiesterase linkages within the oligonucleotide backbone.

Antisense oligonucleotides, siRNAs, shRNAs and ribozymes of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cells and preferably cells expressing SOCS1 and preferably SOCS1 and H3K9-histone methyltransferase SUV39H1. Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and R A virus such as a retrovirus. One can readily employ other vectors not named but known to the art.

Preferred viral vectors are based on non-cytopathic eukaryotic viruses in which nonessential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler, 1990 and in Murry, 1991).

Preferred viruses for certain applications are the adenoviruses and adeno-associated (AAV) viruses, which are double-stranded DNA viruses that have already been approved for human use in gene therapy. Actually 12 different AAV serotypes (AAV1 to 12) are known, each with different tissue tropisms (Wu, Z Mol Ther 2006; 14:316-27). Recombinant AAVs are derived from the dependent parvovirus AAV2 (Choi, VW J Virol 2005; 79:6801-07). The adeno-associated virus type 1 to 12 can be engineered to be replication deficient and is capable of infecting a wide range of cell types and species (Wu, Z Mol Ther 2006; 14:316-27). It further has advantages such as, heat and lipid solvent stability; high transduction frequencies in cells of diverse lineages, including hemopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. See e.g. Sambrook et al, 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate delivery vehicles and micro encapsulation.

The antisense oligonucleotide, siRNA, shRNA or ribozyme or ribozyme encoding nucleic acid sequences according to the invention are generally under the control of a heterologous regulatory region, e.g., a heterologous promoter. The promoter may be specific for Muller glial cells, microglia cells, endothelial cells, pericyte cells and astrocytes, for example, a specific expression in Muller glial cells may be obtained through the promoter of the glutamine synthetase gene is suitable. The promoter can also be, as a matter of example, a viral promoter, such as CMV promoter or any synthetic promoters.

Gene Repression or Disruption of SOCS1 and/or FAS and/or of Suv39h1 and/or 32m

Inhibition of SOCS1, FAS, Suv39h1 and/or 32 in a cell according to the invention may also be effected via repression or disruption of the SOCS1 gene, FAS gene, Suv39h1 gene or B2M gene respectively, such as by deletion, e.g., deletion of the entire gene, exon, or region, and/or replacement with an exogenous sequence, and/or by mutation, e.g., frameshift or missense mutation, within the gene, typically within an exon of the gene. In some embodiments, the disruption results in a premature stop codon being incorporated into the gene, such that the SOCS1, FAS, Suv39h1, or β2m protein is not expressed or is non-functional. The disruption is generally carried out at the DNA level. The disruption generally is permanent, irreversible, or not transient. In some embodiments, inducible and/or reversible gene inactivation of SOCS1 (and/or FAS and/or or Suv39h1 and/or β2m) can be favored.

Well-suited method to edit immune cells for cancer immunotherapy according to the present application are notably described in Lucibello F, Menegatti S, Menger L. “Methods to edit T cells for cancer immunotherapy”. Methods Enzymol. 2020,631:107-135. In some embodiments, the gene disruption or repression is achieved using gene editing agents such as a DNA-targeting molecule, such as a DNA-binding protein or DNA-binding nucleic acid, or complex, compound, or composition, containing the same, which specifically binds to or hybridizes to the gene. In some embodiments, the DNA-targeting molecule comprises a DNA-binding domain, e.g., a zinc finger protein (ZFP) DNA-binding domain, a transcription activator-like protein (TAL) or TAL effector (TALE) DNA-binding domain, a clustered regularly interspaced short palindromic repeats (CRISPR) DNA-binding domain, or a DNA-binding domain from a meganuclease.

Zinc finger, TALE, and CRISPR system binding domains can be “engineered” to bind to a predetermined nucleotide sequence.

In some embodiments, the DNA-targeting molecule, complex, or combination contains a DNA-binding molecule and one or more additional domain, such as an effector domain to facilitate the repression or disruption of the gene. For example, in some embodiments, the gene disruption is carried out by fusion proteins that comprise DNA-binding proteins and a heterologous regulatory domain or functional fragment thereof.

Typically, the additional domain is a nuclease domain. Thus, in some embodiments, gene disruption is facilitated by gene or genome editing, using engineered proteins, such as nucleases and nuclease-containing complexes or fusion proteins, composed of sequence-specific DNA-binding domains fused to, or complexed with, non-specific DNA-cleavage molecules such as nucleases.

These targeted chimeric nucleases or nuclease-containing complexes carry out precise genetic modifications by inducing targeted double-stranded breaks or single-stranded breaks, stimulating the cellular DNA-repair mechanisms, including error-prone nonhomologous end joining (NHEJ) and homology-directed repair (HDR). In some embodiments the nuclease is an endonuclease, such as a zinc finger nuclease (ZFN), TALE nuclease (TALEN), an RNA-guided endonuclease (RGEN), such as a CRISPR-associated (Cas) protein, or a meganuclease. Such systems are well-known in the art (see, for example, U.S. Pat. No. 8,697,359; Sander and Joung (2014) Nat. Biotech. 32:347-355; Hale et al. (2009) Cell 139:945-956; Karginov and Hannon (2010) Mol. Cell 37:7; U.S. Pat. Publ. 2014/0087426 and 2012/0178169; Boch et al. (2011) Nat. Biotech. 29: 135-136; Boch et al. (2009) Science 326: 1509-1512; Moscou and Bogdanove (2009) Science 326: 1501; Weber et al. (2011) PLoS One 6:e19722; Li et al. (2011) Nucl. Acids Res. 39:6315-6325; Zhang et al. (2011) Nat. Biotech. 29: 149-153; Miller et al. (2011) Nat. Biotech. 29: 143-148; Lin et al. (2014) Nucl. Acids Res. 42:e47). Such genetic strategies can use constitutive expression systems or inducible expression systems according to well-known methods in the art.

### ZFPs and ZFNs; TALs, TALEs, and TALENs

In some embodiments, the DNA-targeting molecule includes a DNA-binding protein such as one or more zinc finger protein (ZFP) or transcription activator-like protein (TAL), fused to an effector protein such as an endonuclease. Examples include ZFNs, TALEs, and TALENs. See Lloyd et al., Frontiers in Immunology, 4(221), 1-7 (2013).

In some embodiments, the DNA-targeting molecule comprises one or more zinc-finger proteins (ZFPs) or domains thereof that bind to DNA in a sequence-specific manner. A ZFP or domain thereof is a protein or domain within a larger protein, that binds DNA in a sequence-specific manner through one or more zinc fingers regions of amino acid sequence within the binding domain whose structure is stabilized through coordination of a zinc ion. Generally, sequence-specificity of a ZFP may be altered by making amino acid substitutions at the four helix positions (−1, 2, 3 and 6) on a zinc finger recognition helix. Thus, in some embodiments, the ZFP or ZFP-containing molecule is non-naturally occurring, e.g., is engineered to bind to the target site of choice. See, for example, Beerli et al. (2002) Nature Biotechnol. 20: 135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; Choo et al. (2000) Curr. Opin. Struct. Biol. 10:411-416.

In some embodiments, the DNA-targeting molecule is or comprises a zinc-finger DNA binding domain fused to a DNA cleavage domain to form a zinc-finger nuclease (ZFN). In some embodiments, fusion proteins comprise the cleavage domain (or cleavage half-domain) from at least one Type IIS restriction enzyme and one or more zinc finger binding domains, which may or may not be engineered. In some embodiments, the cleavage domain is from the Type IIS restriction endonuclease Fok I. See, for example, U.S. Pat. Nos. 5,356,802; 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269:31,978-31,982.

In some aspects, the ZFNs efficiently generate a double strand break (DSB), for example at a predetermined site in the coding region of the targeted gene (i.e. Suv39h1). Typical targeted gene regions include exons, regions encoding N-terminal regions, first exon, second exon, and promoter or enhancer regions. In some embodiments, transient expression of the ZFNs promotes highly efficient and permanent disruption of the target gene in the engineered cells. In particular, in some embodiments, delivery of the ZFNs results in the permanent disruption of the gene with efficiencies surpassing 50%. Many gene-specific engineered zinc fingers are available commercially. For example, Sangamo Biosciences (Richmond, CA, USA) has developed a platform (CompoZr) for zinc-finger construction in partnership with Sigma-Aldrich (St. Louis, MO, USA), allowing investigators to bypass zinc-finger construction and validation altogether, and provides specifically targeted zinc fingers for thousands of proteins. Gaj et al., Trends in Biotechnology, 2013, 31(7), 397-405. In some embodiments, commercially available zinc fingers are used or are custom designed. (See, for example, Sigma-Aldrich catalog numbers CSTZFND, CSTZFN, CT11-1KT, and PZD0020).

In some embodiments, the DNA-targeting molecule comprises a naturally occurring or engineered (non-naturally occurring) transcription activator-like protein (TAL) DNA binding domain, such as in a transcription activator-like protein effector (TALE) protein, See, e.g., U.S. Patent Publication No. 20110301073. In some embodiments, the molecule is a DNA binding endonuclease, such as a TALE-nuclease (TALEN). In some aspects the TALEN is a fusion protein comprising a DNA-binding domain derived from a TALE and a nuclease catalytic domain to cleave a nucleic acid target sequence. In some embodiments, the TALE DNA-binding domain has been engineered to bind a target sequence within genes that encode the target antigen and/or the immunosuppressive molecule. For example, in some aspects, the TALE DNA-binding domain may target CD38 and/or an adenosine receptor, such as A2AR.

In some embodiments, the TALEN recognizes and cleaves the target sequence in the gene. In some aspects, cleavage of the DNA results in double-stranded breaks. In some aspects the breaks stimulate the rate of homologous recombination or non-homologous end joining (NHEJ). Generally, NHEJ is an imperfect repair process that often results in changes to the DNA sequence at the site of the cleavage. In some aspects, repair mechanisms involve rejoining of what remains of the two DNA ends through direct re-ligation (Critchlow and Jackson, Trends Biochem Sci. 1998 October; 23(10):394-8) or via the so-called microhomology-mediated end joining. In some embodiments, repair via NHEJ results in small insertions or deletions and can be used to disrupt and thereby repress the gene. In some embodiments, the modification may be a substitution, deletion, or addition of at least one nucleotide. In some aspects, cells in which a cleavage-induced mutagenesis event, i.e. a mutagenesis event consecutive to an NHEJ event, has occurred can be identified and/or selected by well-known methods in the art.

TALE repeats can be assembled to specifically target the Suv39h1 gene. (Gaj et al., Trends in Biotechnology, 2013, 31(7), 397-405). A library of TALENs targeting 18,740 human protein-coding genes has been constructed (Kim et al., Nature Biotechnology. 31, 251-258 (2013)). Custom-designed TALE arrays are commercially available through Cellectis Bioresearch (Paris, France), Transposagen Biopharmaceuticals (Lexington, KY, USA), and Life Technologies (Grand Island, NY, USA). Specifically, TALENs that target CD38 are commercially available (See Gencopoeia, catalog numbers HTN222870-1, HTN222870-2, and HTN222870-3, available on the World Wide Web at www. genecopoeia.com/product/search/detail.php ?prt=26&cid=&key=HTN222870). Exemplary molecules are described, e.g., in U.S. Patent Publication Nos. US 2014/0120622, and 2013/0315884.

In some embodiments the TALENs are introduced as transgenes encoded by one or more plasmid vectors. In some aspects, the plasmid vector can contain a selection marker which provides for identification and/or selection of cells which received said vector.

### RGENs (CRISPR/Cas Systems)

The gene repression can be carried out using one or more DNA-binding nucleic acids, such as disruption via an RNA-guided endonuclease (RGEN), or other form of repression by another RNA-guided effector molecule. For example, in some embodiments, the gene repression can be carried out using clustered regularly interspaced short palindromic repeats (CRISPR) and CRISPR-associated proteins. See Sander and Joung, Nature Biotechnology, 32(4): 347-355.

In general, “CRISPR system” refers collectively to transcripts and other elements involved in the expression of, or directing the activity of, CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

Typically, the CRISPR/Cas nuclease or CRISPR/Cas nuclease system includes a non-coding RNA molecule (guide) RNA, which sequence-specifically binds to DNA, and a CRISPR protein, with nuclease functionality (e.g., two nuclease domains). One or more elements of a CRISPR system can derive from a type I, type II, or type III CRISPR system, such as Cas nuclease. Preferably, the CRISPR protein is a cas enzyme such as9. Cas enzymes are well-known in the field; for example, the amino acid sequence of S. pyogenes Cas9 protein may be found in the SwissProt database under accession number Q99ZW2. In some embodiments, a Cas nuclease and gRNA are introduced into the cell.

In some embodiments, the CRISPR system induces DSBs at the target site, followed by disruptions as discussed herein. In other embodiments, Cas9 variants, deemed “nickases” can be used to nick a single strand at the target site. Paired nickases can also be used, e.g., to improve specificity, each directed by a pair of different gRNAs targeting sequences. In still other embodiments, catalytically inactive Cas9 can be fused to a heterologous effector domain, such as a transcriptional repressor, to affect gene expression.

In general, a CRISPR system is characterized by elements that promote the formation of a CRISPR complex at the site of the target sequence. Typically, in the context of formation of a CRISPR complex, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex. The target sequence may comprise any polynucleotide, such as DNA or RNA polynucleotides. Generally, a sequence or template that may be used for recombination into the targeted locus comprising the target sequences is referred to as an “editing template” or “editing polynucleotide” or “editing sequence”. In some aspects, an exogenous template polynucleotide may be referred to as an editing template. In some aspects, the recombination is homologous recombination.

It is to be noted that in some embodiments, catalytically dead CAS 9 (dCas9) can be used in conjunction with activator or repressor domains to control gene expression.

In some embodiments, one or more vectors driving expression of one or more elements of the CRISPR system are introduced into the cell such that expression of the elements of the CRISPR system direct formation of the CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. In some embodiments, CRISPR system elements that are combined in a single vector may be arranged in any suitable orientation. In some embodiments, the CRISPR enzyme, guide sequence, tracr mate sequence, and tracr sequence are operably linked to and expressed from the same promoter. In some embodiments, a vector comprises a regulatory element operably linked to an enzyme-coding sequence encoding the CRISPR enzyme, such as a Cas protein.

In some embodiments, a CRISPR enzyme in combination with (and optionally complexed with) a guide sequence is delivered to the cell. Typically, CRISPR/Cas9 technology may be used to knockdown gene expression of Suv39h1 in the engineered cells. For example, Cas9 nuclease and a guide RNA specific to the Suv39h1 gene can be introduced into cells, for example, using lentiviral delivery vectors or any of a number of known delivery method or vehicle for transfer to cells, such as any of a number of known methods or vehicles for delivering Cas9 molecules and guide RNAs (see also below).

In some embodiments, inducible gene repression system, notably inducible CRISPR gene inactivation, may be favored such as described in Chylinski, K., Hubmann, M., Hanna, R. E. et al. CRISPR-Switch regulates sgRNA activity by Cre recombination for sequential editing of two loci. Nat Commun 10, 5454 (2019), or in MacLeod, R. S., Cawley, K. M., Gubrij, I. et al. Effective CRISPR interference of an endogenous gene via a single transgene in mice. Sci Rep 9, 17312 (2019).

### Delivery of Nucleic Acids Encoding the Gene Disrupting Molecules and Complexes

In some embodiments, a nucleic acid encoding the DNA-targeting molecule, complex, or combination, is administered or introduced to the cell. Typically, viral and non-viral based gene transfer methods can be used to introduce nucleic acids encoding components of a CRISPR, ZFP, ZFN, TALE, and/or TALEN system to cells in culture.

In some embodiments, the polypeptides are synthesized in situ in the cell as a result of the introduction of polynucleotides encoding the polypeptides into the cell. In some aspects, the polypeptides could be produced outside the cell and then introduced thereto.

Methods for introducing a polynucleotide construct into animal cells are known and include, as non-limiting examples, stable transformation methods wherein the polynucleotide construct is integrated into the genome of the cell, transient transformation methods wherein the polynucleotide construct is not integrated into the genome of the cell, and virus mediated methods.

In some embodiments, the polynucleotides may be introduced into the cell by for example, recombinant viral vectors (e.g. retroviruses, adenoviruses), liposome and the like. Transient transformation methods include microinjection, electroporation, or particle bombardment. The nucleic acid is administered in the form of an expression vector. Preferably, the expression vector is a retroviral expression vector, an adenoviral expression vector, a DNA plasmid expression vector, or an AAV expression vector. In mammalian expression vector, it is to be noted that Promoter driving Cas9 expression can be constitutive or inducible. U6 promoter is typically used for gRNA.

Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Feigner, WO 91/17424; WO 91/16024. Delivery can be to cells (e.g. in vitro or ex vivo administration) or target tissues (e.g. in vivo administration). In some embodiments, Cas9 RNP (ribonucleoproteins) can be used. Cas9 RNPs consist of purified Cas9 protein in complex with a gRNA. They are assembled in vitro and can be delivered directly to cells using standard electroporation or transfection techniques. Cas9 RNPs are capable of cleaving genomic targets with similar efficiency as compared to plasmid-based expression of Cas9/gRNA. Cas9 RNPs are delivered as intact complexes, are detectable at high levels shortly after transfection, and are quickly cleared from the cell via protein degradation pathways. Cas9 RNP delivery to target cells is typically carried out via lipid-mediated transfection or electroporation (see for details Wang, Ming, et al. “Efficient delivery of genome-editing proteins using bioreducible lipid nanoparticles.” Proceedings of the National Academy of Sciences 113.11 (2016): 2868-2873; Liang, Xiquan, et al. “Rapid and highly efficient mammalian cell engineering via Cas9 protein transfection.” Journal of biotechnology 208 (2015): 44-53; Zuris, John A., et al. “Cationic lipid-mediated delivery of proteins enables efficient protein-based genome editing in vitro and in vivo.” Nature biotechnology 33.1 (2015): 73-80 or Kim, Sojung, et al. “Highly efficient RNA-guided genome editing in human cells via delivery of purified Cas9 ribonucleoproteins.” Genome research 24.6 (2014): 1012-1019).

RNA or DNA viral-based systems include retroviral, lentivirus, adenoviral, adeno-associated and herpes simplex virus vectors for gene transfer.

For a review of gene therapy procedures, see Anderson, Science 256:808-813 (1992); Nabel & Feigner, TIBTECH 11:211-217 (1993); Mitani & Caskey, TIBTECH 11: 162-166 (1993); Dillon. TIBTECH 11: 167-175 (1993); Miller, Nature 357:455-460 (1992); Van Brunt, Biotechnology 6(10): 1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Bohm (eds) (1995); and Yu et al., Gene Therapy 1: 13-26 (1994).

A reporter gene which includes but is not limited to glutathione-5-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP), may be introduced into the cell to encode a gene product which serves as a marker by which to measure the alteration or modification of expression of the gene product.

### Cell Preparation

Isolation of the cells includes one or more preparation and/or non-affinity based cell separation steps according to well-known techniques in the field. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some embodiments, the cell preparation includes steps for freezing, e.g., cryopreserving, the cells, either before or after isolation, incubation, and/or engineering. Any of a variety of known freezing solutions and parameters in some aspects may be used.

Typically, the cells are incubated prior to or in connection with genetic engineering and/or SOCS1 (and/or Suv39h1 and/or FAS and/or β2m) inhibition.

The incubation steps can comprise culture, incubation, stimulation, activation, expansion and/or propagation.

In some embodiments, inhibition of SOCS1 as per the invention (and/or of Suv39h1 and/or FAS, and/or of β2m in some embodiments) may also be achieved in vivo after injection the cells to the targeted patients. Typically, inhibition of SOCS1 can be performed using pharmacological inhibitors as previously described.

In other embodiments, inhibition of SOCS1 (and/or of Suv39h1, and/or FAS, and/or β2m in some embodiments) as per the method as previously described can also be performed during stimulation, activation and/or expansion steps. For example, PBMCs, or purified T cells, or purified NK cells, or purified lymphoid progenitors, are expanded in vitro in presence of the pharmacological inhibitor(s) of SOCS1 and/or FAS and/or Suv39h1 and/or β2m before adoptive transfer to patients. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a genetically engineered antigen receptor.

The incubation conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the stimulating conditions or agents include one or more agent, e.g., ligand, which is capable of activating an intracellular signaling domain of a TCR complex. In some aspects, the agent turns on or initiates TCR/CD3 intracellular signaling cascade in a T cell. Such agents can include antibodies, such as those specific for a TCR component and/or costimulatory receptor, e.g., anti-CD3, anti-CD28, for example, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/ml). In some embodiments, the stimulating agents include 1L-2 and/or IL-15, for example, an IL-2 concentration of at least about 10 units/mL.

In some aspects, incubation is carried out in accordance with techniques such as those described in U.S. Pat. No. 6,040,177 to Riddell et al., Klebanoff et al., J Immunother. 2012; 35(9): 651-660, Terakura et al., Blood. 2012; 1:72-82, and/or Wang et al. J Immunother. 2012, 35(9):689-701.

In some embodiments, the T cells are expanded by adding to the culture-initiating composition feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some aspects, the feeder cells are added to culture medium prior to the addition of the populations of T cells.

In some embodiments, the stimulating conditions include temperature suitable for the growth of human T lymphocytes, for example, at least about 25 degrees Celsius, generally at least about 30 degrees, and generally at or about 37 degrees Celsius. Optionally, the incubation may further comprise adding non-dividing EBV-transformed lymphoblastoid cells (LCL) as feeder cells. LCL can be irradiated with gamma rays in the range of about 6000 to 10,000 rads. The LCL feeder cells in some aspects is provided in any suitable amount, such as a ratio of LCL feeder cells to initial T lymphocytes of at least about 10: 1.

In embodiments, antigen-specific T cells, such as antigen-specific CD4+ and/or CD8+ T cells, are obtained by stimulating naive or antigen specific T lymphocytes with antigen. For example, antigen-specific T cell lines or clones can be generated to cytomegalovirus antigens by isolating T cells from infected subjects and stimulating the cells in vitro with the same antigen.

In some aspects, the methods include assessing expression of one or more markers on the surface of the engineered cells or cells being engineered. In one embodiment, the methods include assessing surface expression of one or more target antigen (e.g., antigen recognized by the genetically engineered antigen receptor) sought to be targeted by the adoptive cell therapy, for example, by affinity-based detection methods such as by flow cytometry.

### Vectors and Methods for Cell Genetic Engineering

In some aspects, the genetic engineering involves introduction of a nucleic acid encoding the genetically engineered component or other component for introduction into the cell, such as a component encoding a gene-disruption protein or nucleic acid.

Generally, the engineering of CARs into immune cells (e.g., T cells) requires that the cells be cultured to allow for transduction and expansion. The transduction may utilize a variety of methods, but stable gene transfer is required to enable sustained CAR expression in clonally expanding and persisting engineered cells.

In some embodiments, gene transfer is accomplished by first stimulating cell growth, e.g., T cell growth, proliferation, and/or activation, followed by transduction of the activated cells, and expansion in culture to numbers sufficient for clinical applications.

Various methods for the introduction of genetically engineered components, e.g., antigen receptors, e.g., CARs, are well known and may be used with the provided methods and compositions. Exemplary methods include those for transfer of nucleic acids encoding the receptors, including via viral, e.g., retroviral or lentiviral, transduction, transposons, and electroporation.

In some embodiments, recombinant nucleic acids are transferred into cells using recombinant infectious virus particles, such as, e.g., vectors derived from simian virus 40 (SV40), adenoviruses, adeno-associated virus (AAV). In some embodiments, recombinant nucleic acids are transferred into T cells using recombinant lentiviral vectors or retroviral vectors, such as gamma-retroviral vectors (see, e.g., Koste et al. (2014) Gene Therapy 2014 Apr. 3.; Carlens et al. (2000) Exp Hematol 28(10): 1137-46; Alonso-Camino et al. (2013) Mol Ther Nucl Acids 2, e93; Park et al., Trends Biotechnol. 2011 November; 29(11): 550-557.

In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), spleen focus forming virus (SFFV), or adeno-associated virus (AAV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3: 102-109.

Methods of lentiviral transduction are also known. Exemplary methods are described in, e.g., Wang et al. (2012) J. Immunother. 35(9): 689-701; Cooper et al. (2003) Blood. 101: 1637-1644; Verhoeyen et al. (2009) Methods Mol Biol. 506: 97-114; and Cavalieri et al. (2003) Blood. 102(2): 497-505.

In some embodiments, recombinant nucleic acids are transferred into T cells via electroporation {see, e.g., Chicaybam et al, (2013) PLoS ONE 8(3): e60298 and Van Tedeloo et al. (2000) Gene Therapy 7(16): 1431-1437). In some embodiments, recombinant nucleic acids are transferred into T cells via transposition (see, e.g., Manuri et al. (2010) Hum Gene Ther 21(4): 427-437; Sharma et al. (2013) Molec Ther Nucl Acids 2, e74; and Huang et al. (2009) Methods Mol Biol 506: 115-126). Other methods of introducing and expressing genetic material in immune cells include calcium phosphate transfection (e.g., as described in Current Protocols in Molecular Biology, John Wiley & Sons, New York. N.Y.), protoplast fusion, cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)).

Other approaches and vectors for transfer of the genetically engineered nucleic acids encoding the genetically engineered products are those described, e.g., in international patent application, Publication No.: WO2014055668, and U.S. Pat. No. 7,446,190.

### Composition of the Invention

The present invention also includes compositions containing the cells as described herein and/or produced by the provided methods. Typically, said compositions are pharmaceutical compositions and formulations for administration, such as for adoptive cell therapy.

A pharmaceutical composition of the invention generally comprises at least one engineered immune cell of the invention and a pharmaceutically acceptable carrier.

As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can further be incorporated into the compositions. In some aspects, the choice of carrier in the pharmaceutical composition is determined in part by the particular engineered CAR or TCR, vector, or cells expressing the CAR or TCR, as well as by the particular method used to administer the vector or host cells expressing the CAR. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001 to about 2% by weight of the total composition.

A pharmaceutical composition is formulated to be compatible with its intended route of administration.

### Therapeutic Methods

The present invention also relates to the cells as previously defined for their use in adoptive therapy (notably adoptive T cell therapy), typically in the treatment of cancer in a subject in need thereof. In some embodiments, the cells as herein disclosed can be used in allogenic transfers notably in the case of cells defective for SOCS1 and/or FAS optionally in combination with inactivation of Suv39h1 and/or β2m.

Treatment”, or “treating” as used herein, is defined as the application or administration of cells as per the invention or of a composition comprising the cells to a patient in need thereof with the purpose to cure, heal, alleviate, relieve, alter, remedy, ameliorate, improve or affect the disease such as cancer, or any symptom of the disease (e.g., cancer). In particular, the terms “treat’ or treatment” refers to reducing or alleviating at least one adverse clinical symptom associated with the disease such as the cancer cancer, e.g., pain, swelling, low blood count etc.

With reference to cancer treatment, the term “treat’ or treatment” also refers to slowing or reversing the progression neoplastic uncontrolled cell multiplication, i.e. shrinking existing tumors and/or halting tumor growth. The term “treat’ or treatment” also refers to inducing apoptosis in cancer or tumor cells in the subject.

The subject of the invention (i.e. patient) is a mammal, typically a primate, such as a human. In some embodiments, the primate is a monkey or an ape. The subject can be male or female and can be any suitable age, including infant, juvenile, adolescent, adult, and geriatric subjects. In some embodiments, the subject is a non-primate mammal, such as a rodent. In some examples, the patient or subject is a validated animal model for disease, adoptive cell therapy, and/or for assessing toxic outcomes such as cytokine release syndrome (CRS). In some embodiments of the invention, said subject has a cancer, is at risk of having a cancer, or is in remission of a cancer.

The cancer may be a solid cancer or a “liquid tumor” such as cancers affecting the blood, bone marrow and lymphoid system, also known as tumors of the hematopoietic and lymphoid tissues, which notably include leukemia and lymphoma. Liquid tumors include for example acute myelogenous leukemia (AML), chronic myelogenous leukemia (CML), acute lymphocytic leukemia (ALL), and chronic lymphocytic leukemia (CLL), (including various lymphomas such as mantle cell lymphoma, non-Hodgkins lymphoma (NHL), adenoma, squamous cell carcinoma, laryngeal carcinoma, gallbladder and bile duct cancers, cancers of the retina such as retinoblastoma).

Solid cancers notably include cancers affecting one of the organs selected from the group consisting of colon, rectum, skin, endometrium, lung (including non-small cell lung carcinoma), uterus, bones (such as Osteosarcoma, Chondrosarcomas, Ewing's sarcoma, Fibrosarcomas, Giant cell tumors, Adamantinomas, and Chordomas), liver, kidney, esophagus, stomach, bladder, pancreas, cervix, brain (such as Meningiomas, Glioblastomas, Lower-Grade Astrocytomas, Oligodendrocytomas, Pituitary Tumors, Schwannomas, and Metastatic brain cancers), ovary, breast, head and neck region, testis, prostate and the thyroid gland.

In some embodiments, the subject is suffering from or is at risk of an infectious disease or condition, such as, but not limited to, viral, retroviral, bacterial, and protozoal infections, immunodeficiency, Cytomegalovirus (CMV), Epstein-Barr virus (EBV), adenovirus, BK polyomavirus. In some embodiments, the disease or condition is an autoimmune or inflammatory disease or condition, such as arthritis, e.g., rheumatoid arthritis (RA), Type I diabetes, systemic lupus erythematosus (SLE), inflammatory bowel disease, psoriasis, scleroderma, autoimmune thyroid disease, Grave's disease, Crohn's disease multiple sclerosis, asthma, and/or a disease or condition associated with transplant

The present invention also relates to a method of treatment and notably an adoptive cell therapy, preferably an adoptive T cell therapy, comprising the administration to a subject in need thereof of a composition a previously described.

In some embodiments, the cells or compositions are administered to the subject, such as a subject having or at risk for a cancer or any one of the diseases as mentioned above. In some aspects, the methods thereby treat, e.g., ameliorate one or more symptom of, the disease or condition, such as with reference to cancer, by lessening tumor burden in a cancer expressing an antigen recognized by the engineered cell.

Methods for administration of cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

In some embodiments, the cell therapy, e.g., adoptive cell therapy, e.g., adoptive T cell therapy, is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject. In such embodiments, the use of cells defective for SOCS1 and/or FAS, optionally in combination with SUV39h1 and/or β2m inactivation is favored.

Administration of at least one cell according to the invention to a subject in need thereof may be combined with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cell populations are administered prior to the one or more additional therapeutic agents. In some embodiments, the cell populations are administered after to the one or more additional therapeutic agents.

With reference to cancer treatment, a combined cancer treatment can include but is not limited to chemotherapeutic agents, hormones, anti-angiogens, radiolabelled compounds, immunotherapy, surgery, cryotherapy, and/or radiotherapy.

Immunotherapy includes but is not limited to immune checkpoint modulators (i.e. inhibitors and/or agonists), monoclonal antibodies, cancer vaccines.

Preferably, administration of cell in an adoptive T cell therapy according to the invention is combined with administration of immune checkpoint modulators, notably checkpoint inhibitors. Checkpoint inhibitors include, but are not limited to, PD-1 inhibitors, PD-L1 inhibitors, Lag-3 inhibitors, Tim-3 inhibitors, TIGIT inhibitors, BTLA inhibitors, V-domain Ig suppressor of T-cell activation (VISTA) inhibitors and CTLA-4 inhibitors, IDO inhibitors for example. Co-stimulatory antibodies deliver positive signals through immune-regulatory receptors including but not limited to ICOS, CD137, CD27 OX-40 and GITR. Most preferably, the immune checkpoint modulators comprise a PD-1 inhibitor (such as anti-PD-1), a PDL1 inhibitor (such as antiPDL1) and/or a CTLA4 inhibitor.

In addition or as an alternative to the combination with checkpoint blockade, the immune cell (notably the immune cell composition) of the present disclosure may also be genetically modified to render them resistant to immune-checkpoints using gene-editing technologies including but not limited to TALEN and Crispr/Cas. Such methods are known in the art, see e.g. US20140120622. Gene editing technologies may be used to prevent the expression of immune checkpoints expressed by T cells including but not limited to PD-1, Lag-3, Tim-3, TIGIT, BTLA CTLA-4 and combinations of these. The immune cell as discussed here may be modified by any of these methods.

The immune cell according to the present disclosure may also be genetically modified to express molecules increasing homing into tumours and or to deliver inflammatory mediators into the tumour microenvironment, including but not limited to cytokines, soluble immune-regulatory receptors and/or ligands.

The present invention also relates to the use of a composition comprising the engineered immune cell as herein described for the manufacture of a medicament for treating a cancer, an infectious disease or condition, an autoimmune disease or condition, or an inflammatory disease or condition in a subject.

The present invention also encompasses a method for the manufacture of a universal immune cell, in particular universal T cell, usable in allogenic adoptive therapy, for example in the treatment of cancer, comprising a step of repressing of FAS and/or SOCS1 activity (at the gene, mRNA or gene level as previously described), in a T cell optionally in combination with inactivation of Suv39h1 and/or β2m.

The present invention also encompasses a method for allogenic adoptive therapy, notably for allogenic cancer adoptive therapy, notably allogenic ATCT comprising steps of:
obtaining at least one immune cell, from a subject
modifying said at least one immune cell to inactivate Fas and/or SOC1
administrating said at least one immune cell, typically in the form of a pharmaceutical composition, to another subject in need thereof;
optionally wherein said at least one immune cell is further modified to express one or more genetically modified antigen receptor(s) as previously described;
optionally wherein said at least one immune cell is further modified to inactivate Suv39h1 and/or β2m;
optionally wherein the at least one cell is a CD4+ T cell, or a mixed population of CD4+/CD8+ T cells as previously described.

This method can also be combined with the embodiments previously described.

### FIGURES

FIG. 1: In vivo genome-scale (18400 genes) CRISPR pooled screens identify SOCS1 as non-redundant inhibitor of Antigen-experienced (Ag-exp) CD4 T cell expansion during an ongoing immune response.

(A) Two cohorts experimental design to assess naive and Ag-exp CD4 T cell expansion in the course of an ongoing immune response used in B-D. (B) Flow plots and percentage (percentage highlighted are from singlets live CD45.1⁺ CD4 T cells) of proliferating Marilyn CD4 T cells, either 10⁶ naive or Ag-exp in vitro, during an ongoing immune response in C57BL/6 mice. Mice were injected with 106 cells intravenously and primed in vivo by injection of 10⁶ Dby peptide-loaded LPS-matured DCs into the footpad. (C, D) Survival and IL2 production of CD45.1 Ag-exp CD4 T cells compared to naive CD45.1 Marilyn CD4 T cells during a recall response in vivo. (E) Ag-exp Cas9-Marilyn CD4 T cells CD44/CD62L phenotype and lentiviral library transduction efficiency (BFP+), prior to puromycin selection and injection in vivo. (F) Scatter plot comparing sgRNA normalized read counts in the original plasmid DNA library and in the transduced T cells after 4 days of puromycin selection (5 μg/mL). (G) Representative flow plots and quantification of proliferating CD45.1-library-transduced Cas9-Marilyn CD4 T cells compared to CD45.1-Mock-transduced Cas9-Marilyn CD4 T cells. Mice were injected with 12·10⁶ CD4 T cells IV and primed with 4·10⁶ Dby peptide-loaded LPS-matured DCs in the footpad at day 0 and day 7. (H) Enriched hits in the CFSE^{lo} subset of CD45.1-library-transduced CD4 T cells compared to the CFSE^{hi} subset in an ongoing immune response in vivo. (I) Representative plots and percentage (gated on singlets live CD45.1+CD4 T cells) of proliferating Ag-exp Mock Marilyn or sgSOCS1 Marilyn cells during a recall response, at day 14. Mice were injected with 2·10⁶ CD4 T cells IV and primed with 10⁶ peptide-pulsed LPS-matured DCs at day 0 and day 7. (G, H) Data shown are from two independents primary GW screens. (H) p-value corresponds to the gene-level enriched p-value and log 2 fold change (LFC) to the median LFC of all sgRNA supporting the enriched RRA score. Targets with an FDR<0.5 are highlighted in black. Each point is an individual mouse, open symbols are replicates from independent experiments (FP: footpad, DC: dendritic cells, pept: peptide, Ag-exp: antigen-experienced).

FIG. 2: SOCS1 is a node integrating several cytokines signals to actively silence polycytokine release. (A) SORTing strategy of CFSE^{lo} (green) and CFSE^{hi} (red) naive or Ag-exp Marilyn cells from an ongoing immune response. (B) Heat map displaying the expression of a selected list of cytokine receptors by proliferating or inhibited Marilyn cells (first seven receptors p<0.01, FDR<0.5). (C) Representative flow plots (percentage highlighted are from singlets live CD45.1+CD4 T cells) and quantification of 10⁶ Marilyn naive IFNγ-R^{+/-} or Marilyn Ag-exp IFNγ-R^{+/-} or Ag-exp IFNγ-R^{-/-} expansion in vivo after cells transfer and footpad vaccinations at day 14, with or without (w/o) cohort 1 expansion. (D) Representative flow plots (percentage highlighted are from singlets live CD45.1+CD4 T cells) and quantification of 10⁶ Marilyn Ag-exp expansion in vivo during a recall response, in the presence of blocking antibodies (200 μg) injected intraperitoneally at day 7, day 9, day 11: isotypes, anti-IL2RP, anti-IFNγRα. (E) Flow cytometric evaluation of CD69, CD25, IRF4 and expression in sgSOCS1 Ag-exp Marilyn compared to Mock cells after overnight co-culture with peptide-pulsed LPS-matured DCs in vitro. (F) Flow plots and percentage of IFN-γ-, TNFα- and IL-2-producing Mock or sgSOCS1 Marilyn. Values are shown as means or means±SD. Each point is an individual mouse, open symbols are replicates from independent experiments, analyzed by Mann-Whitney U tests or two-way ANOVA (E).

FIG. 3: Ag-exp sgSocs1 Marilyn CD4 T cells acquires a polyfunctional Th-Cytotoxic phenotype and enhances the rejection of male bladder MB49 tumors.

(A) Schematic of Marilyn CD4 T cells (ACT) in C57BL/6 female mice-bearing the male DBY-expressing bladder tumor line MB49. (B) Tumor-free survival following ACT, log-rank (Mantel-Cox) test. (C) Growth curves of MB49 tumors in C57BL6 mice following the different ACT: PBS control, adoptive transfer of 10⁶ mock Ag-exp Marilyn or 10⁶ sgSOCS1 Ag-exp Marilyn Cas9, in mice receiving anti-CD8a and anti-Asialo GM1 (anti-GM1)-depleting antibodies. (D) Representative flow plots and quantification of Mock or sgSOCS1 Marilyn cells in the tumor draining lymph node (TdLN), in the tumor and in the irrelevant lymph nodes (irr-LN) at day 7 after ACT. (E) Representative flow plots and percentage of mock and sgSOCS1 Marilyn cells proliferation in the TdLN at day 7 after ACT. (F) Gene set enrichment analysis (GSEA) of selected hallmarks transcriptional signatures (MSigDB) with an FDR value<0.05 in Ag-exp sgSOCS1 versus Ag-exp mock Marilyn T cells in the TdLN (n=3 replicates from 2 pooled mice). (G) Differentially expressed genes in Tumor draining lymph node (TdLN)-infiltrating CD45.1 Marilyn sgSOCS1 cells compared to Marilyn mock cells. Transcripts with an FDR value<0.05 are highlighted in light green. (H) Representative flow plots and quantification of IFNγ+IL2+ and IFNγ⁺ TNFα⁺-producing mock or sgSOCS1 Marilyn CD4 T cells in the TdLN at day 7 after transfer. (I) Representative flow plots of MHC-II molecules expressed by MB49 tumors. (J) Flow plot and quantification of granzyme B (GZMB) expressed by tumor-infiltrating sgSOCS1 Marilyn CD4 T cells at day 7. Data are shown as mean, analyzed by Mann-Whitney U tests, from two independent experiments, n=4-6 mice/group.

FIG. 4: B16-OVA tumor rejection with improved ACT: Socs1 gene inactivation restores the proliferation of OT2 cells and enhances OT1 cell survival and cytotoxicity. (A) Schematic of OT1 CD8− and OT2 CD4− adoptive T cell therapy (ACT) in C57BL/6 mice-bearing B16-OVA melanoma tumors. (B) Growth curves of B16-OVA tumors in C57BL6 mice following adoptive transfer with OT1 (2·10⁶ Mock or 2·10⁶ sgSOCS1) and OT2 cells (2·10⁶ Mock or 2·10⁶ sgSOCS1). (C) Kaplan-Meier survival analysis of B16-OVA-bearing mice following ACT, log-rank (Mantel-Cox) test. (D) Representative plots and quantification of Mock or sgSOCS1 OT1 and OT2 cells in the tumor draining lymph node (TdLN), in the tumor or in the irrelevant lymph nodes (Irr-LN) at day 7 after ACT, gated on singlets live Vα2⁺ T cells. (E) Representative flow plots and percentage of Mock or sgSOCS1 OT1 and OT2 cells proliferating in the TdLN at day 7. (F, G) Representative flow plots and quantification of mock or sgSOCS1 OT2 and OT1 tumor-infiltrating cells producing IFN-γ and granzyme B molecules and at day 7 after transfer. Data are shown as mean, analyzed by Mann-Whitney U tests, from two independent experiments, n=5-8 mice/group.

FIG. 5: SOCS1 inactivation restores CAR4 T cell expansion in vivo and boosts CAR8 T cell efficacy in controlling B-ALL disease.

(A) Schematic of CAR-T cell engineering and adoptive T-cell therapy (ATCT) with 2·10⁶ CD4 CAR (CAR4) and 2·10⁶ CD8 CAR (CAR8) T cells of NALM6-Luc-bearing mice. (B) CAR expression assessed using CD19/Fc fusion protein and central memory phenotype prior to NSG injection. (C, D) Representative flow plots and quantification of bone marrow infiltration with CAR4 and CAR8 mock and sgSOCS1 in NALM6-Luc bearing NSG mice at day 7 and day 28 after transfer, gated on singlets live HLA-I*, CD45.2⁻ mouse cells. (E) Heat map of selected differentially expressed genes (FDR<0.05) between mock and sgSOCS1 CAR T cells related to activation (red), proliferation/survival (blue) and effector functions (green) at day 7 after transfer. (F) Gene set enrichment analysis of the transcriptional signatures from hallmarks signatures in CAR4/8 sgSOCS1 versus CAR4/8 mock (n=6 mice). (G, H) Representative flow plots and quantification of effector molecules produced by CAR T cells from infiltrated BM at day 28. (I) NALM6-LUC tumor growth after ATCT with 2·10⁶ CAR4/8 mock or 2·10⁶ CAR4 sgSOCS1/8mock or 2·10⁶ CAR4mock/8sgSOCS1 or 2·10⁶ CAR4/8 sgSOCS1 as detailed in FIG. 5A. (J) Kaplan-Meier analysis of survival of NSG mice, log-rank (Mantel-Cox) test. (K) NALM6-LUC-bearing mice were treated with 4·10⁶ CAR-T cells. Tumor burden shown as bioluminescent signal quantified per animal over a 35-day period, n=5 mice/group. Data are represented as mean, analyzed by Mann-Whitney U tests, from two independents experiments (n=5-6 mice/group).

FIG. 6: In vivo genome-scale (18400 genes) CRISPR pooled screens identify Fas and B2m as non-redundant targets allowing T cell survival in MHC-mismatched hosts

(A, B) Representative flow plots and absolute number of live CD45.1 (H2-Kb) Marilyn CD4 T cells in the spleen of fully immunocompetent C57BL6 (syngeneic) and BALB/c (allogeneic) mice, 4 days after intravenous (IV) injections. (C) Schematics of in vivo genome-wide CRISPR screening design. (D, E) Representative flow plots and absolute number of live CD45.1 mock or library-mutated Marilyn CD4 T cells in the spleen of fully immunocompetent C57BL6 and BALB/c mice, 4 days after IV injections of 107 CD4 T cells. (F) Meta-analysis of in vivo genome-wide CRISPR pooled screens of library-mutated Marilyn cells survival in BALB/c mice as compared to diversity from C57BL6 infiltrated mice, using MAGeCK analysis. (G) Gene-level representation of significant individual sgRNA distributions across each experiment, showing enriched genes (Fas, B2m) in BALB/c mice as compared to C57BL6 mice. (H) Representative flow plots and quantification of spleen infiltrated by mock (expressing the congenic marker CD45.1/2) and Fas-inactivated Marilyn CD4 T cells (sgFas, CD45.1/1) co-injected in C57BL6 mice (syngeneic) or BALB/c mice (allogeneic), 4 days after IV injections. (I) Percentage of Fas negative Marilyn cells in C57BL6 mice and BALB/c mice. (J) Representative flow plots and quantification of spleen infiltrated by mock (expressing the congenic marker CD45.1/2) and B2m-inactivated Marilyn CD4 T cells (sgB2m, CD45.1/1) co-injected in C57BL6 mice or BALB/c mice, 4 days after IV injections. (K) Percentage of B2m negative Marilyn cells in C57BL6 mice and BALB/c mice. Data are shown as mean, analyzed by Mann-Whitney U tests, from two independent experiments, n=3-6 mice/group.

FIG. 7: Fas targeting improves resistance to both T-cell and NK-mediated allogeneic rejection and can be potentiated in vivo by Socs1-inactivation

(A) Schematics of experiment design for fully MHC-mismatched rejection of C57BL6 T cells in BALB/c mice (screen model). (B, C) Representative flow plots and absolute number of live CD45.1 polyclonal (CD4 and CD8) T cells in the spleen of fully immunocompetent C57BL6 and BALB/c mice, 4 days after IV injections of 2·10⁶ T cells, in mice receiving either IgG or anti-CD8a (200 ug/per day) or anti-AsialoGM1 (anti-GM1, 30 ug/per day)-depleting antibodies. (D) Pre-injection expression of Fas in polyclonal CD45.1 T cells (H2-Kb), 4 days after electroporation with sgRNA and HIFI-Cas9. (E, F) Percentage of indels in polyclonal CD45.1 T cells (H2-Kb) electroporated with sgSOCS1 (E) or with sgSIOCS1 and Fas (F), using Tide analysis. (G) Representative flow plots and absolute number of live polyclonal CD45.1 T cells (H2-Kb) in the spleen of fully immunocompetent C57BL6 and BALB/c mice, 4 days after IV injections of 2·10⁶ T cells. (H) Inactivated polyclonal CD45.1 T cells fold change survival as compared to Mock cells in syngeneic and allogeneic mice. (I) Schematics of experiment design for semi-allogeneic rejection of F1 T cells (C57BL6×BALB/c) in C57BL6 mice. (J) Number of F1 Marilyn cells in the spleen of C57BL6 mice 4 days after IV injections of 2·10⁶ cells, in mice receiving either IgG or anti-CD8a (200 ug/per day) or anti-NK1.1 (30 ug/per day)-depleting antibodies. (K) Expressions of H2-Kb et H2-Kd in CD45.1 F1 Marilyn CD4 T cells from C57BL6 mice treated with anti-NK1.1. (L, M) Representative flow plots and absolute number of F1 Marilyn CD4 T cells survival in C57BL6 mice, 4 days after IV injections of 2·10⁶ F1 cells. Data are shown as mean, analyzed by Mann-Whitney U tests, from two independent experiments (G, H), n=3-6 mice/group.

FIG. 8: Fas and SOCS1 dual inactivation protects murine and human tumor-reactive T cells from alloimmune cellular rejection in vivo

(A) Schematics of murine immunocompetent model to assess CD4 or CD8 tumor-specific T cells functionality across MHC barriers. (B) Representative flow plots and absolute number of CD45.1 F1 OT1 cells infiltrating the spleen of B16-OVA bearing C57BL6 mice, 15 days after IV injections with 2·10⁶ F1 OT1 cells. (C) Representative flow plots and absolute number of CD45.1 F1 OT1 cells infiltrating the tumor and expressing granzyme B (GZB+) of B16-OVA bearing C57BL6 mice, 15 days after IV injections with 2·10⁶ F1 OT1 cells. (D) Schematics of experiment design using human CAR-T cells. (E) CAR-T cells pre-injection phenotype showing the composition in CD4 and CD8 T cells, the expression of CD19-CARbbz and TCRb after electroporation by sgTRAC. (F) Expression of Fas in engineered CAR-T cells by flow, 4 days after electroporation. (G) Relative expression of SOCS1 mRNA assessed by RT-qPCR in TRAC/FAS/SOCS1-inactivated A2-CAR-T cells as compared to TRAC-inactivated A2-CAR-T cells, 4 days after electroporation. (H) Representative flow plots of HLA, A, B, C+ cells in the bone marrow (BM) of NSG mice, 15 days after IV injections with 2·10⁶ TRAC-inactivated A2-CAR-T cells. (I, J, K) Number of A2-CAR-T cells (I), Nalm6-sgB2m (J) and A2+ T cells (K) infiltrating NSG mice bone marrow, 15 days after CAR-T cells injection. Data are shown as mean, n=3-5 mice/group.

### EXAMPLES

### Material and Methods

### Cell Lines and Mice

B16-OVA and MB49 cell lines, kindly provided by E. Piaggio and C. Thery, FFLuc-BFP NALM6 (NALM6) cell line, provided by O. Bernard were maintained in RPMI-1640 supplemented with 10% FBS. CD45.1 and CD45.2 female Marilyn TCR-transgenic Rag2^{-/-} mice, specific for the HY male antigen were crossed to Rosa26-Cas9-EGFP knock-in mice (026179, Jackson lab). Thy1.1 and Thy1.2 OT-II TCR-transgenic Rag2^{-/-} mice, CD45.1 female OT-I TCR-transgenic Rag2^{-/-} mice, specific for OVA and female, male NOD-scid IL2Rg^{-/-} (NSG) mice were also used in this study. Female C57BL/6 mice were purchased from Charles River Laboratories (L'Arbresle, France). All experiments were conducted with 6-12 weeks old mice, in an accredited animal facility by the French Veterinarian Department following ethical guidelines, approved by the relevant ethical committee (AP AF1S #6030-20 16070817147969 v2, authorisation #XX DAP 2017-023).

### Cell Culture and Adoptive Transfers

Naive CD4+ T cells were obtained from peripheral lymph nodes of Marilyn or OT-II mice. Antigen experienced CD4⁺ T cells were generated in vitro by priming lymph nodes and splenocytes of CD45.1 Marilyn mice or Thy1.1 OT-II mice with respectively 10 nM Dby (NAGFN-SNRANSSRSS, Genscript) and 5 μM OVAII peptide (InvivoGen). IL-2 (10 ng/mL), IL-7 (2 ng/mL) (Peprotech) were added starting at day 4 and every 3 days in complete RPMI-1640 supplemented with 10% FBS and 0.55 mM β-mercaptoethanol. Ag-exp OT-I cells from lymph nodes and spleen were cultured with 0.5 μM SIINFEKL (InvivoGen) and maintained with IL15 (50 ng/mL) (Peprotech) every two days. T cells were labeled with 5 μM CFSE (Invitrogen) in PBS for 8 minutes at 37° C. For in vivo GS screen, 4·10⁶ naive CD45.2 Marilyn CD4⁺ T cells were transferred and footpad vaccinated with 4·10⁶ Dby loaded-LPS-matured bone marrow derived-dendritic cells (BMDCs). Seven days later, 12·10⁶ library transduced or 12·10⁶ Mock-transduced CD45.1 Cas9-Marilyn cells were injected intravenously and mice were at the same time footpad-vaccinated with 4·10⁶ Dby-loaded-LPS-matured BMDCs. For validation experiments, a first cohort of 10⁶ naive CD45.2 Marilyn or Thy1.2 OT-II cells was transferred into CD45.2 B6 hosts footpad vaccinated with 10⁶ peptide-loaded-LPS-matured BMDCs. After 7 days, a second cohort of either 10⁶ naive CD45.1 Marilyn, Thy1.1 OT-558 II cells or 2·10⁶ Ag-exp CD45.1 Marilyn, Thy1.1 OT-II CD4⁺ T cells were injected and mice were footpad-vaccinated with 10⁶ peptide-loaded-LPS-matured BMDCs. BMDCs were generated by 10 days culture in complete IMDM containing 20 ng/ml of GM-CSF (Peprotech) and maturation was induced by a 20-hour treatment with 1 ug/mL lipopolysaccharide (Sigma-Aldrich), pulsed with 50 nM Dby or 20 μM OVAII peptide for 2 hours. Mice were treated with blocking antibodies from Bioxcell, including isotypes control rat IgG2b (clone LTF2), IgG2a (clone 2A3), anti-mouse CD122 antibody (clone TM-Beta1), anti-mouse IFN-gR (clone GR-20), intraperitoneally on day 7, 11 and day 11 after ACT (10 mg/kg). For adoptive cell therapies, female C57BL6 host were subcutaneously implanted with either 1.5·10⁶ male bladder MB49 tumor cells or 4·10⁵ B16-OVA melanoma cells. At day 10 for the MB49 model and on day 7 for B16-OVA, 106 Marilyn CD4⁺ T cells or 2·10⁶ OT-I and 2·10⁶ OT-II cells were adoptively transferred into tumor-bearing mice (n=4-6/group). Mice were sacrificed when the tumors exceeded 15 mm in diameter for the B16-OVA model.

Peripheral blood mononuclear cells (PBMCs) from healthy donors were isolated by density gradient centrifugation. T lymphocytes were purified using the Pan T cell isolation kit (Miltenyi Biotech) and activated with Dynabeads Human T-Activator CD3/CD28 (1:1 beads:cell) (ThermoFisher) in X-vivo 15 medium (Lonza) supplemented with 5% human serum (Sigma) and 0.5 mM β-mercaptoethanol at density of 106 cells/mL. 48 hours after activation, T cells were transduced with lentiviral supernatants of an anti-CD19(FMC63)-CD8tm-41BB-CD3ζ CAR construct (rLV EF119BBz, Flash Therapeutics) at MOI 10. Two days later, the CD3/CD28 beads were magnetically removed, CAR T cells were electroporated with Cas9-ribonucleoproteins (Cas9-RNP) and maintained in X-vivo supplemented with IL7 (5 ng/mL) and IL15 (5 ng/mL). Six days after electroporation, CD4⁺ and CD8+ CAR-T cell were separated using CD8⁺ T Cell Isolation kit (Miltenyi) for mutagenesis quantification on gDNA and western blot analysis of SOCS1 expression. Male or female 8-12-week-old NSG mice were injected with 4·10⁵ NALM6 cells intravenously by tail vein injection. Three days later, 2·10⁶ CAR T cells were administered intravenously by tail vein injection (day 0). Tumor burden was measured by bioluminescence imaging using the Lumina IVIS Imaging System (PerkinElmer). Mice were sacrificed when the radiance was >5·10⁶ [p/s/cm≤s/sr].

### Cytotoxicity Assays

The cytotoxicity of T cells transduced with a CAR was determined by co-culturing in triplicates at the indicated E/T ratio, CAR T cells (Effectors) with Nalm6 cells (Targets) in a total volume of 100 μl per well in X-vivo medium. The maximal luciferase expression (relative light units; RLUmax) was determined with target cells alone plated at the same cell density. 18 h later, 100 μl luciferase substrate (Perkin Elmer) was directly added to each well. Luminescence was detected using a SpectraMax 1D3 plate reader (VWR). Lysis was determined as (1−(RLUsample)/(RLUmax))×100.

### Antibodies and Flow Cytometry Analysis

Lymph nodes cells, splenocytes and tumor samples enriched on a density gradient medium (Histopaque, Sigma) were incubated with murine antibodies (STAR methods). Human cultured cells, bone marrow cells and splenocytes from NSG mice cells were stained with the indicated Abs or soluble protein: fluorochrome-conjugated antibodies specific for human (STAR methods). The intracellular staining was performed either with intracellular staining permeabilization wash buffer (BD Bioscience) or Foxp3 kit (eBioscience). CAR expression was assessed using 9269-CD-050 Recombinant Human CD19 Fc Chimera Protein (Bio Techne), at 4° C. for one hour, at 1/100 dilution. Viability was evaluated using Fixable Viability Dye eFluor 780 (eBioscience) or Aqua Live dead (Thermo Fisher). Re-stimulation was performed with 20 ng/mL of PMA (Sigma), 1 μM of ionomycin (Sigma) and BD Golgi plug for 4 hours at 37° C. Cell Sorting Set-up Beads (Life Technologies) were used to quantify and normalized cell number between samples and experiments. Stainings were performed in a blocking solution: 5% FCS, and 2% anti-FcR 2.4G2, and samples acquired on a LSRII/Fortessa (BD) and analyzed with FlowJo software (V10, Tree Star). Cell sorting was performed on ARIAII (BD).

### Western Blot Analysis

T cells (2·10⁶) were lysed using RIPA lysis buffer (Thermofisher) and 1×Protease Inhibitor Cocktail (Sigma). Cell debris were removed by centrifugation at 14,000 rpm for 15 min at 4° C. and 20-40 μg of proteins from the supernatant were separated using SDS-PAGE and transferred to a PVDF membrane. SOCS1 and p-actin (loading control) were visualized using monoclonal antibodies anti-SOCS1 (1 μg/mL) (ab62584; Abcam), anti-Actin mouse (Millipore, clone C4), HRP-anti-Rabbit IgG1 (Cell Signaling Technology). HRP-anti mouse IgG (Cell signaling) on Chemidoc Touch Imaging system (Biorad). Signal intensity was quantified with ImageJ software.

### Genome-Wide CRISPR-Cas9 Screens

The lentiviral gRNA plasmid library for genome-wide CRISPR-Cas9 screen (Mouse Improved Genome-wide Knockout CRISPR Library v2, Pooled Library #67988 #) and mock vector (#67974) was obtained from Addgene. The library was amplified following the protocol provided by Addgene. Briefly, 4×25 ul of NEB 10-beta Electrocompetent E. coli (NEB, cat. no. C3020K) were electroporated with of 4×10 ng/μl and cultured in 4×500 mL of ampicillin-treated Luria-Bertani (LB) incubate at 37° C. overnight with shaking. The plasmids were extracted with 12 columns of EndoFree plasmid Maxi kit (Qiagen). To prepare the virus library, 293T cells at low passage (<7) in 20 cm dish (X15) were transfected with 11 μg of gRNA library, 11 μg of psPAX2 and 2.5 μg of pVSV-G. Twenty-four hours after transfection, the medium was changed to DMEM-1% BSA, collected at 48h, 60h and 72h, then centrifuged, filtered through 0.45 uM PVDF membranes (Millipore), concentrated using Amicon Ultra 15 ml centrifugal filters (Merck) and used fresh. One day before T cells transduction, CD4⁺ T cells are enriched using MagniSort Mouse CD4⁺ T cell Enrichment Kit (Thermofisher scientific) and seeded at a density of 1.5. 106 cells/ml with. fresh medium and. culture medium supplemented with IL-2 (10 ng/ml), IL-7 (2 ng/ml). Cells are spinfected for 90 min, at 32° C., 900 g with 10 ug/ml of protamine sulfate (Sigma) and 8 ug/ml of DEAE-dextran (Sigma). The volume of the lentivirus library used is the one required for achieving an optimal transduction efficiency, MOI of 0.3 after 5 days selection with 5 ug/ml of puromycin (Sigma). CFSEhi and CFSElo Cas9-CD45.1 Marilyn CD4⁺ T cells were sorted and their gDNA extracted using 10 μl of lysis buffer-AL (Qiagen-DNeasy blood and tissue kit), 1 μl proteinase K (Qiagen), followed by 30 min incubation at 56° C., 30 min incubation at 95° C. and resuspension in 20 μl of ddH20 on ice. The gRNAs were amplified by a two-step PCR method using the Herculase II Fusion DNA Polymerase (Agilent). For the first step PCR, all the gDNA extracted is used to perform approximately 30×50-μl PCR reactions with the forward primer 50 bp-F and the reverse primer 50 bp-R (STAR methods); the PCR program used is 94° C. for 180 s, 16 cycles of 94° C. for 30 s, 60° C. for 10 s and 72° C. for 25 s, and a final 2-min extension at 68° C.

Products of the first-step PCR are pooled, purified with Ampure XP (Agencourt) and quantified using the dsDNA HS assay kit. Three 50-μl PCR reactions were performed with the forward primer Index-F and one of the reverse primers (Index-R1 to R6). The PCR program used is 94° C. for 180 s, 18 cycles of 94° C. for 30 s, 54° C. for 10 s and 72° C. for 18 s, and a final 2-min extension at 68° C.

Products of the second-step PCR reactions were purified and analysed with Caliper Labchip for DNA samples (HT DNA High Sensitivity LabChip Kit; Perkin Elmer) prior to sequencing with the Miseq or HiSeq2500 instrument for the library representation (Illumina). The DNA quality was assessed and quantified using an Agilent DNA 1000 series II assay and a Qubit fluorometer (Invitrogen).

Sequencing was performed with a 10% Phix control, using the 25-bp single-end sequencing protocol preceded by 23 dark cycles to mark the repetitive structure of the target region.

Bulk rnRNA Sequencing and Analysis

Between 10⁴ and 3·10⁴ murine and human T cells were sorted from lymph nodes and tumors in TCL buffer (Qiagen) with 1% of b-mercaptoethanol. Total RNA was purified using the Single Cell RNA purification kit (Norgen) according to the manufacturer's instructions, including a step of DNAse treatment (Qiagen). The RNA integrity number was then evaluated with an Agilent RNA 6000 pico kit. cDNA synthesis and Illumina-compatible libraries were generated from total RNA (0.25-10 ng) by Next Generation Sequencing platform of the Institut Curie, using SMARTer Stranded Total RNA-Seq Kit-Pico Input Mammalian according to manufacturer's instructions. Libraries were then sequenced on an Illumina NovaSeq-S1 using 100 bp paired-end mode (OR HiSeq-Rapid Run-PE100). FASTQ files were mapped to the reference genome hg19 (human) or mm10 (mice) using Hisat2 and counted by featureCounts from the Subread R package to produce read count tables. EdgeR was then used to normalize read counts and gene with expression >0.5 cpm in at least three replicates were kept for subsequent analysis.

Differential gene expression was performed with limma-voom R package. The fgsea R package was used to compute the enrichment scores. For Affymetrix analysis, gene expression was conducted using Mouse Clariom D chip (Thermo Fisher). RNA samples were amplified with Ovation Pico WTA System v2 (Nugen) and labeled with Encore biotin module (Nugen). Array were hybridized with 5 μg of labeled DNA and assayed on a GeneChip Scanner 3000 7G (Affymetrix). Raw data were generated and controlled with Expression console (Affymetrix) at the Institut Curie Genomic facility.

### Genome-Wide Data Processing

FASTQ files obtained after sequencing were demultiplexed using the HiSeq Analysis software (Illumina). MAGeCK (Li et al., 2014) count command was then used to generate per-sgRNA read count table by matching single-end reads with sgRNA sequences from the genome-scale sgRNA Yusa library (Koike-Yusa et al., 2014). Before mapping, the library was first cleansed of (i) all sgRNA that did not map the reference genome (here mm10) and (ii) all sgRNA that mapped multiple spot in the reference genome (multihits). Redundant sgRNA were merged. A normalizing factor for each sample was then calculated using Trimmed Mean of M-values (TMM) method implemented in edgeR R package (Robinson and Oshlack, 2010) Normalized counts were filtered for low expressed sgRNA (keeping only sgRNA with at least 4 count per million in 3 samples) and transformed to log 2-counts per million using voom implemented in limma R package. Differential expression of each sgRNA was calculated using ImFit function in limma using the high and low CFSE cell fraction from each screen.

For each sgRNA, enriched and depleted p-values were computed using one-tailed paired Student's t tests. From these, Robust Rang Aggregation (RRA) score (10.1093/bioinformatics/btr709) for each gene was computed among multiple sgRNAs (n=5) of each gene and gene-level related p values and corresponding adjusted p-values [False Discovery Rates (FDR)] were obtained using a permutation test with 1,000,000 iterations with same size randomized gene sets. Finally, graphical representation of genes according to their enriched p value and median log fold change of sgRNA supporting the RRA score was done.

### Cas9-RNP Validations

1 μl Oligos crRNA (100 nM) and 1 μl tracrRNA (100 nM) (STAR Methods) for murine T cells and 1 μl Oligos crRNA1+1 μl Oligos crRNA2+1 μl Oligos tracrRNA for human T cells were annealed at 95° C. for 5 min and incubated at room temperature 10 min with 10 μg S.p Hifi Cas9 Nuclease V3 (STAR Methods). 2·10⁶ T cells were resuspended in 20 μl of nucleofection solution with 3 μl or 4 μl RNP and transferred to Nucleofection cuvette strips (4D-Nucleofector X kit S; Lonza). Murine T cells were electroporated using the DN110 program of 4D nucleofector (4D-Nucleofector Core Unit: Lonza, AAF-1002B), human CAR T cells using the program E0115. T cells were then incubated at 32° C. for 24 to 48 hours to increase the mutagenesis efficacy (Doyon et al., 2010), prior to resuspension in supplemented fresh medium. Murine CD4⁺ T cells were maintained in complete RPMI with IL2 (10 ng/mL) and IL-7 (2 ng/mL). Human T cells were maintained in X-Vivo with 5% human serum and IL7 (5 ng/mL) and IL15 (5 ng/mL). Locus-specific PCRs (STAR Methods) were performed on genomic DNA and frequencies of NHEJ mutations were assessed by sequencing (Eurofins, Mix2seq) and TIDE analysis (https://tide.deskgen.com).

### Statistical Analysis

One-way ANOVA, two-way ANOVA, or Mann-Whitney non-parametric test with p<0.05 were performed using Prism 8.0 software (GraphPad). Multiple comparisons were corrected with the Bonferroni coefficient and Kaplan-Meier survival curves were compared with the log-rank test.

### Results

1) SOCS1 as a Major Intrinsic Checkpoint of T Cells and Notably CD4+ T Cells

### In Vivo Genome-Wide Screen Identified SOCS1 as a Major Non-Redundant Inhibitor of Antigen Experienced CD4⁺ T-Cell Expansion

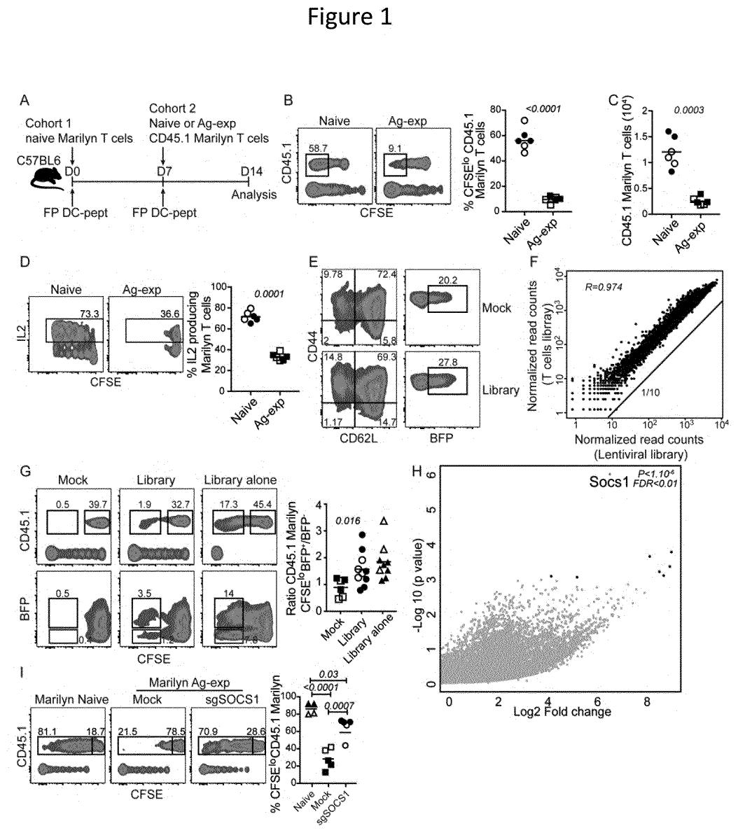

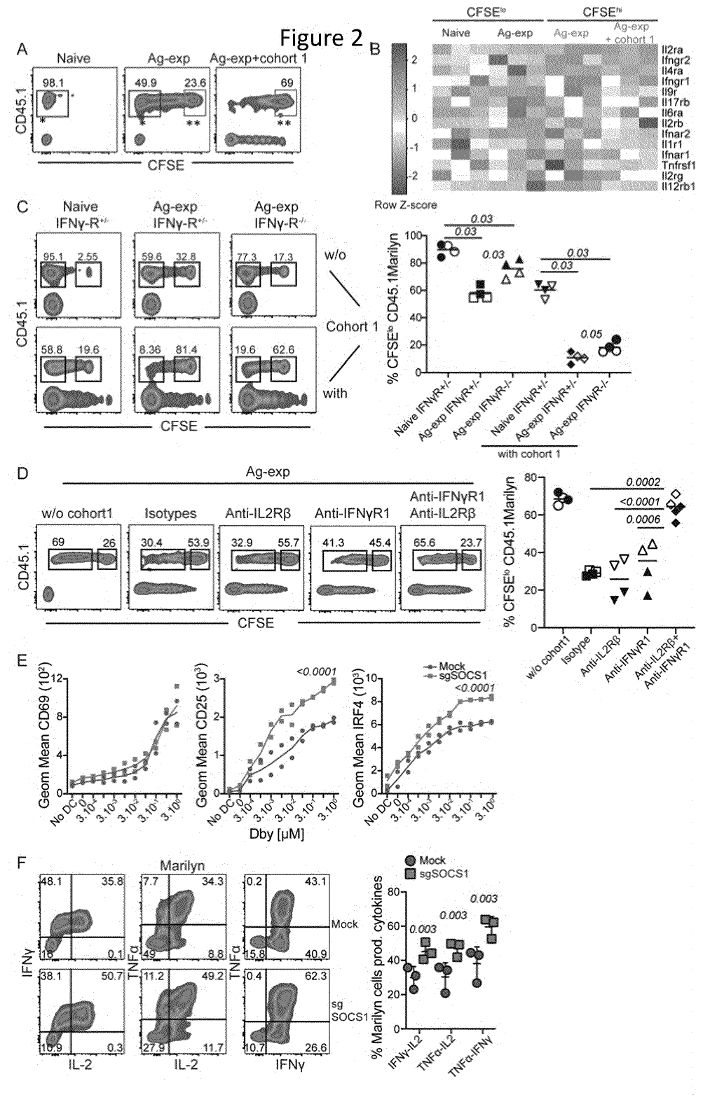

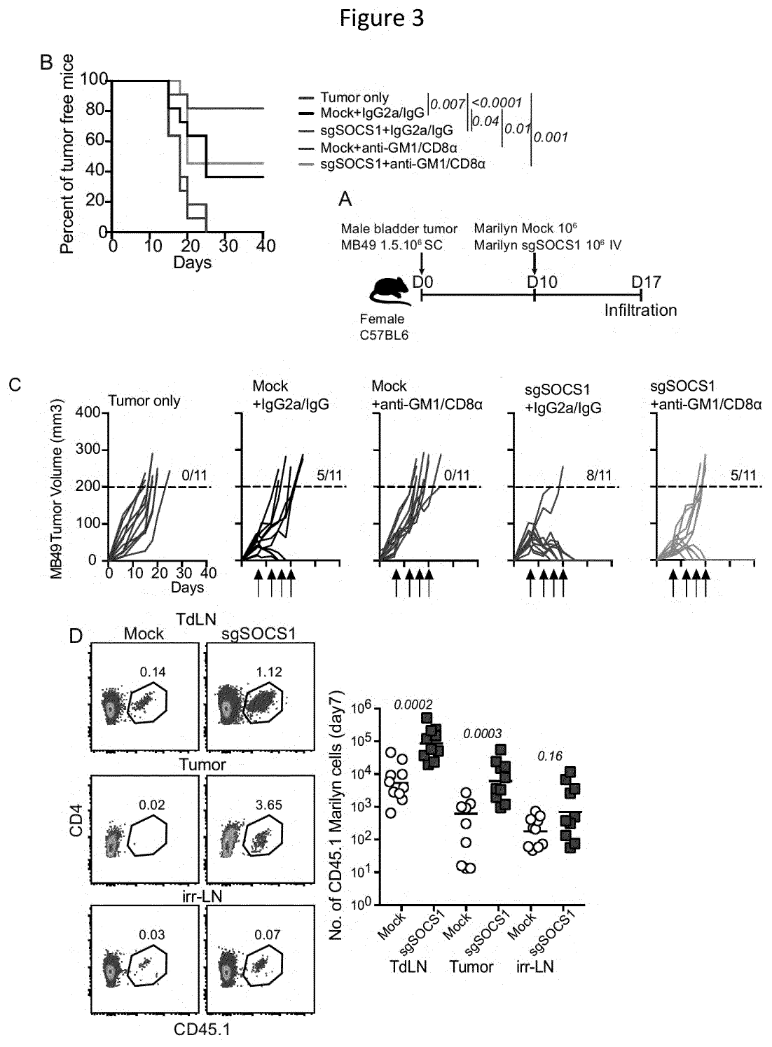

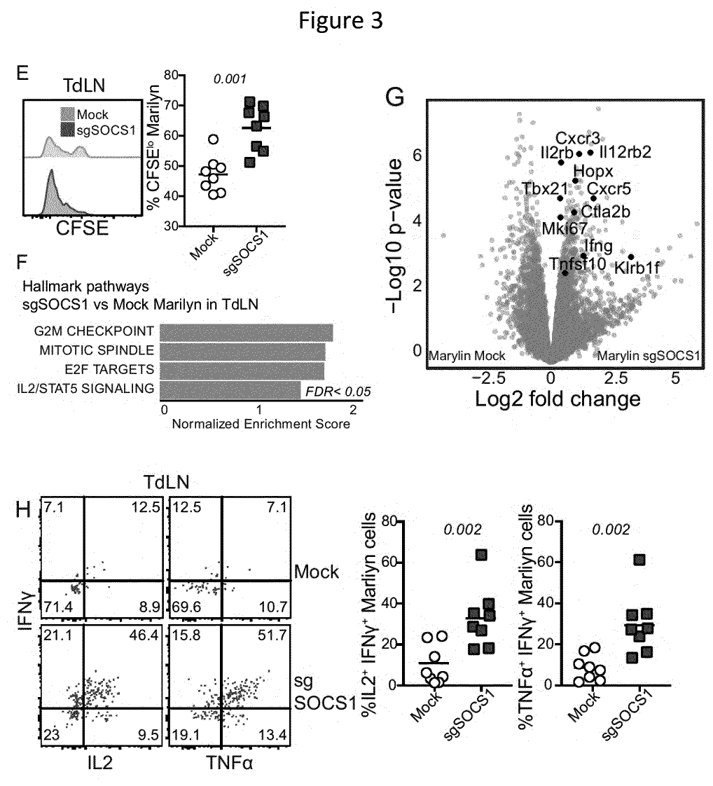

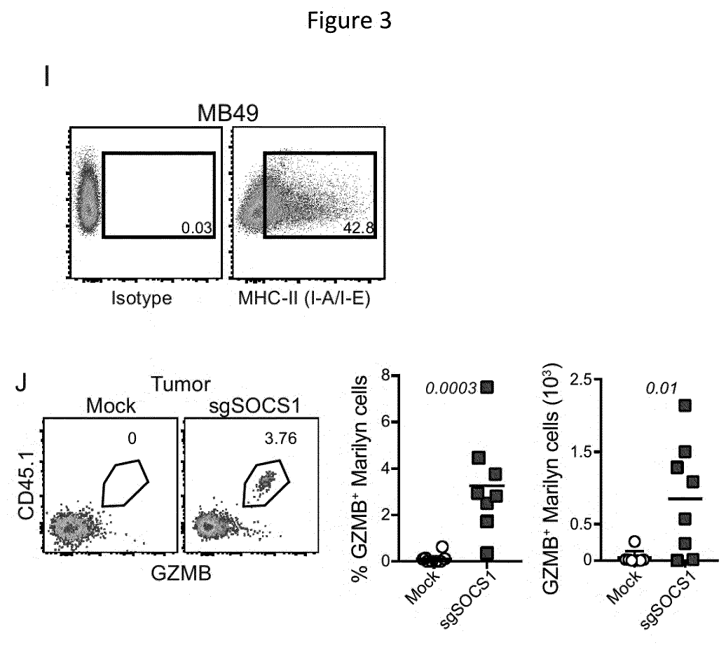

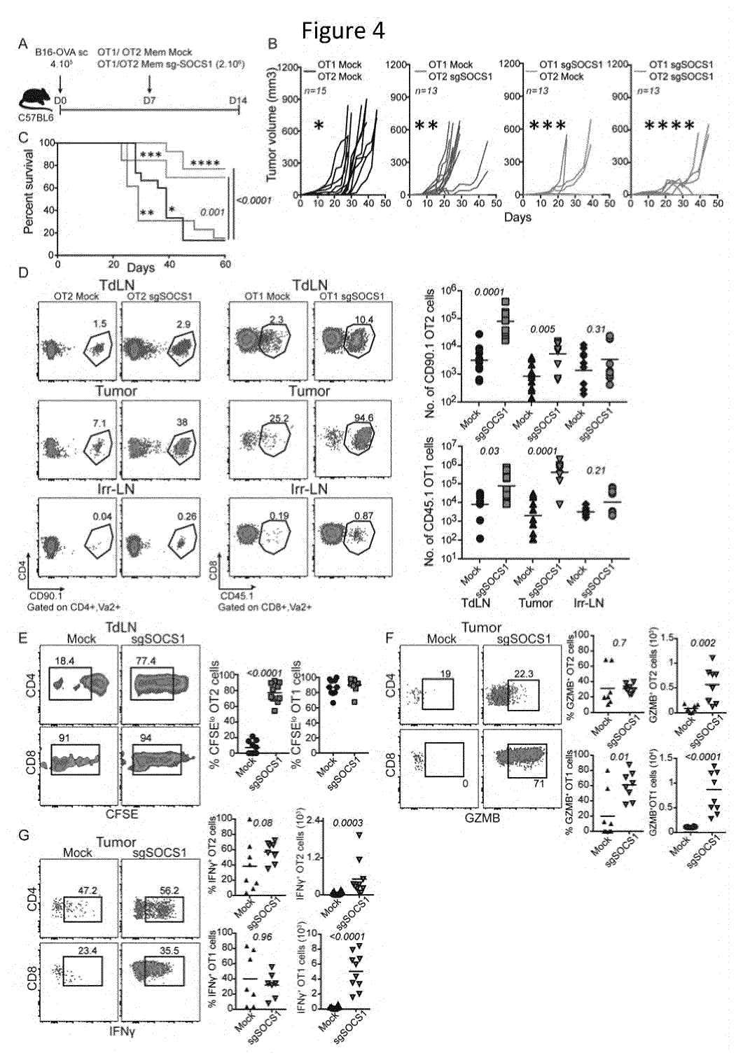

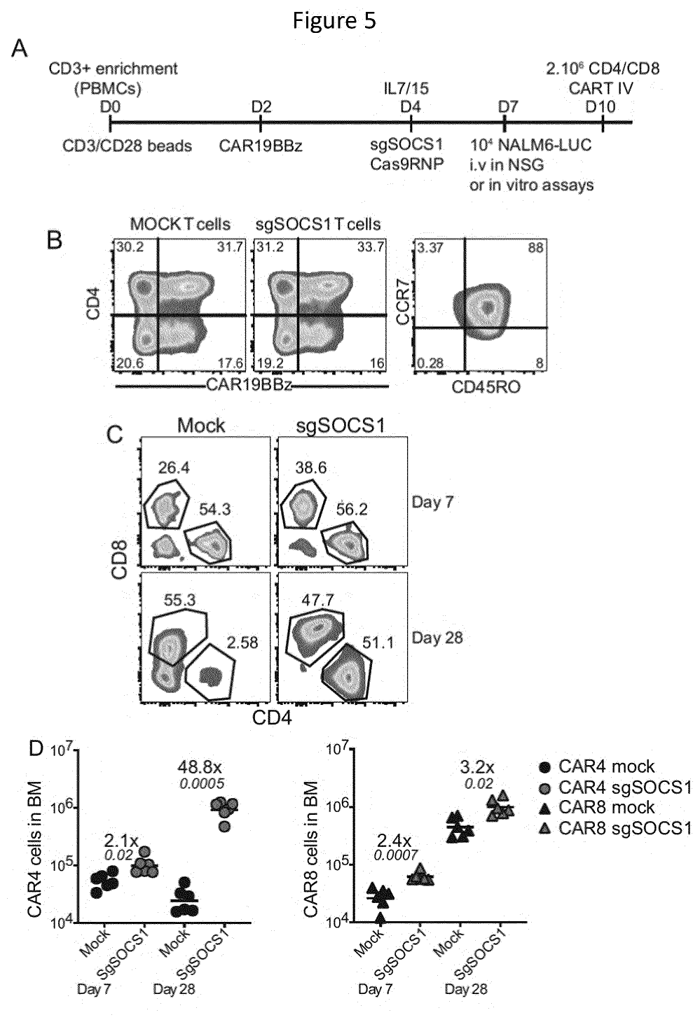

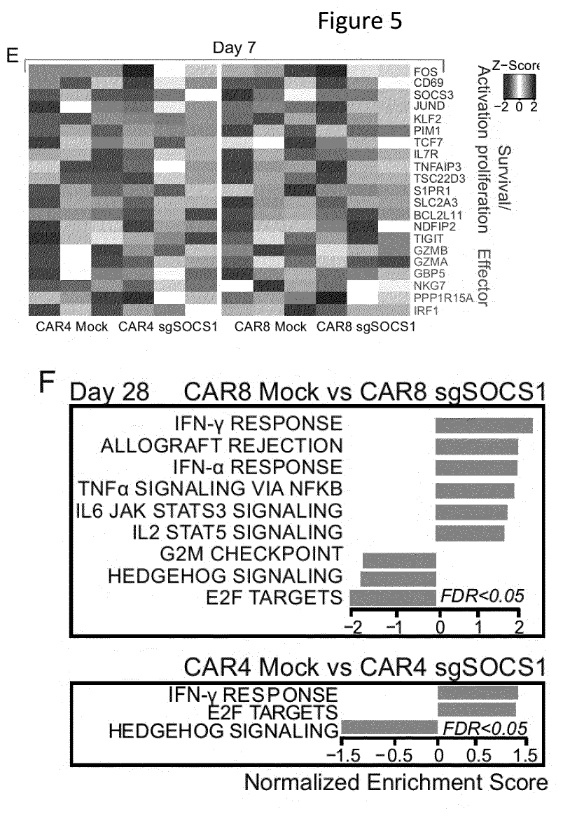

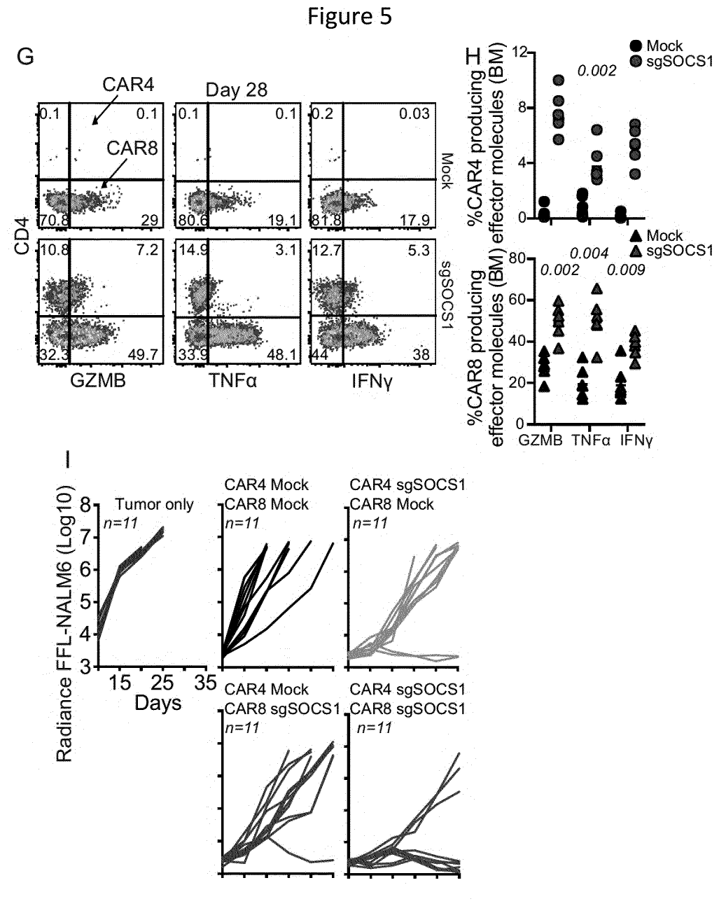

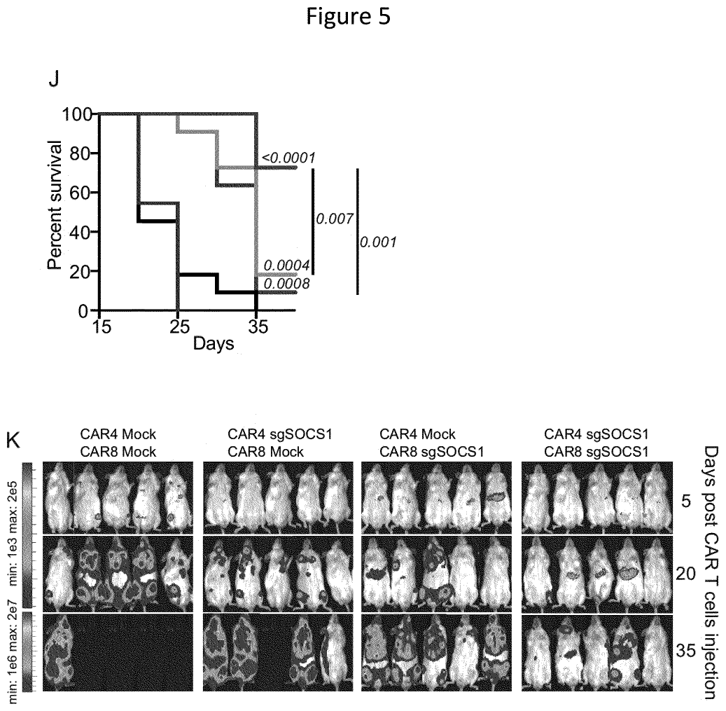

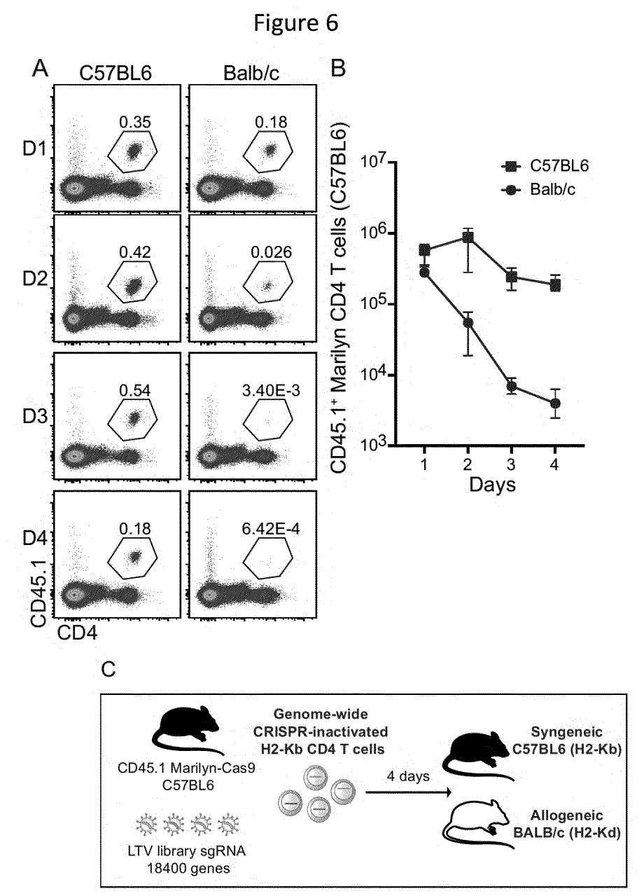

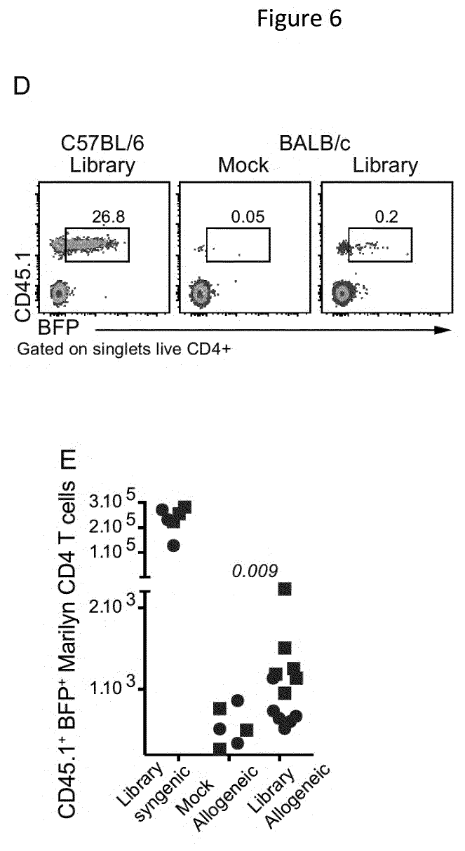

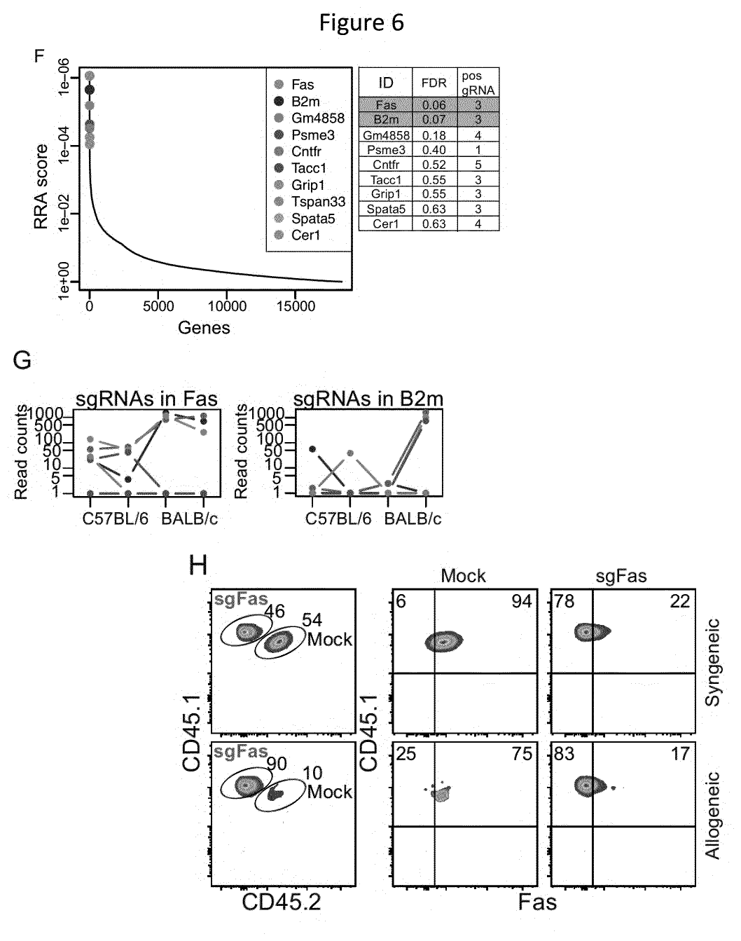

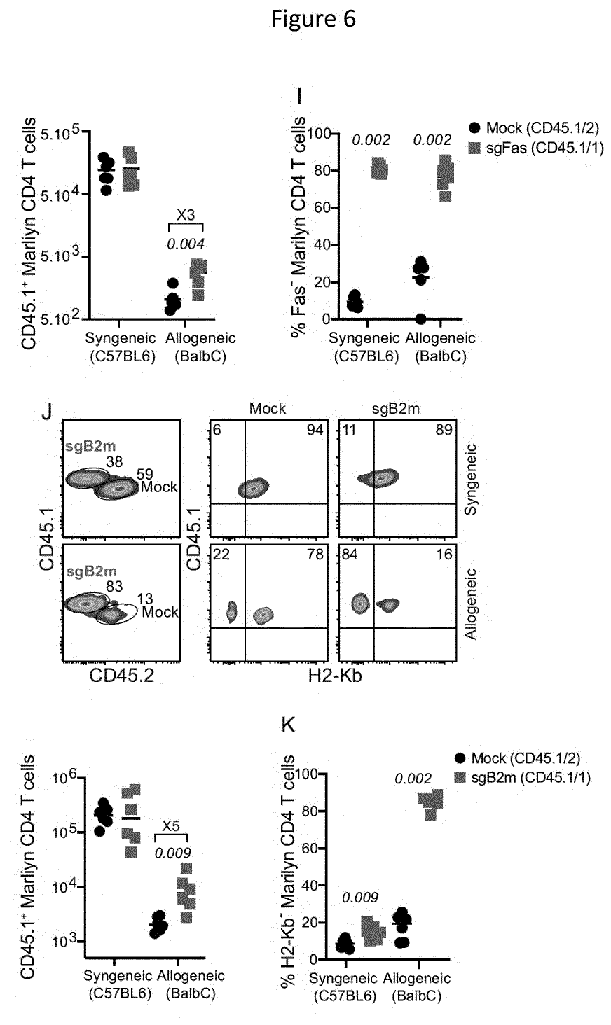

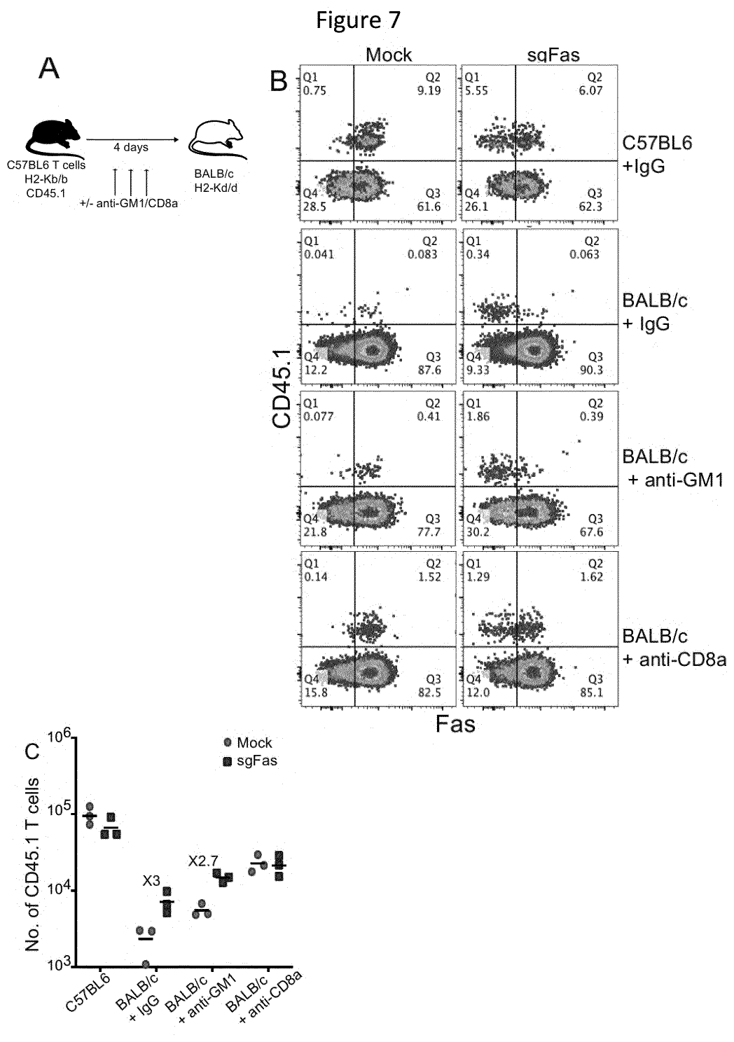

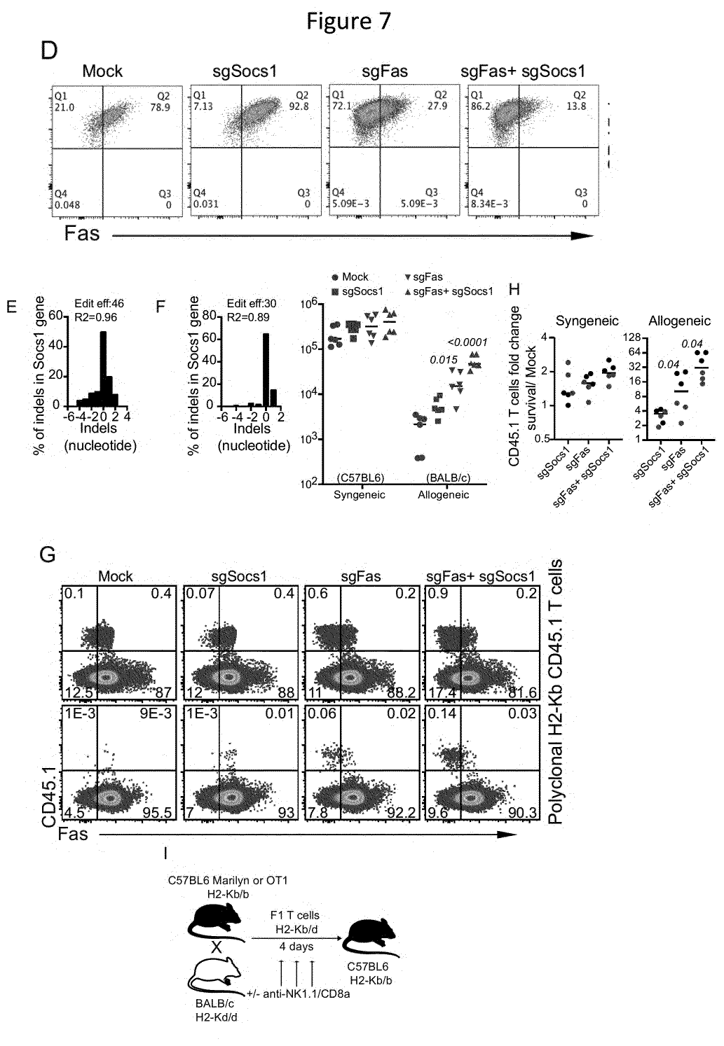

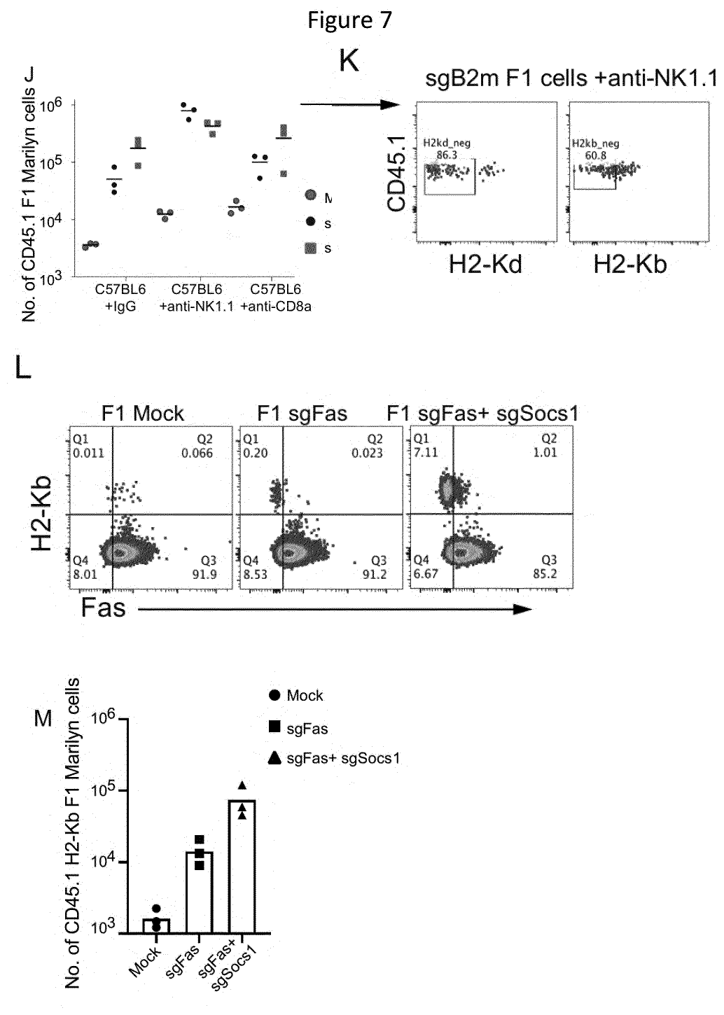

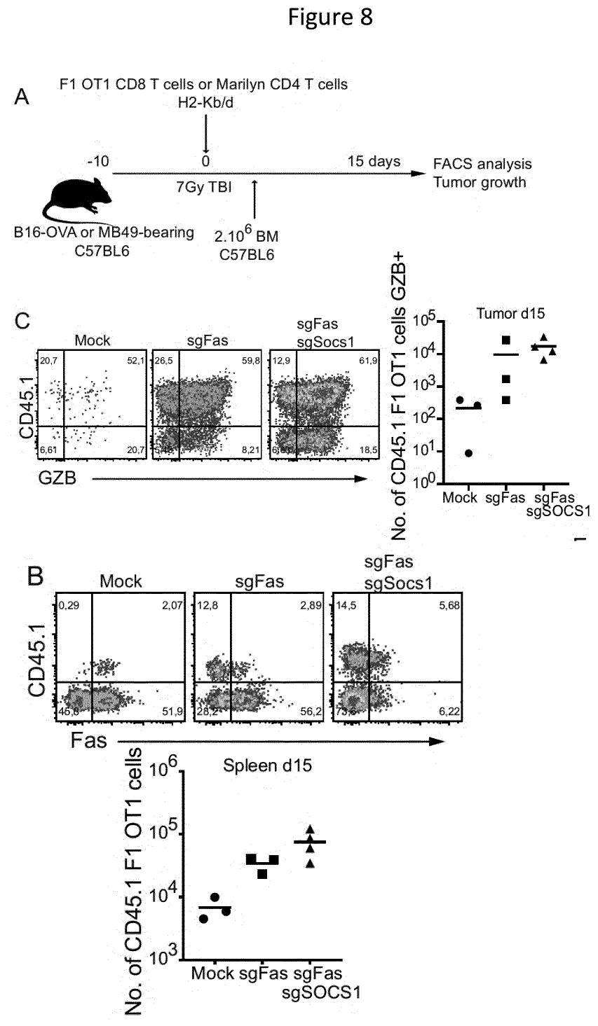

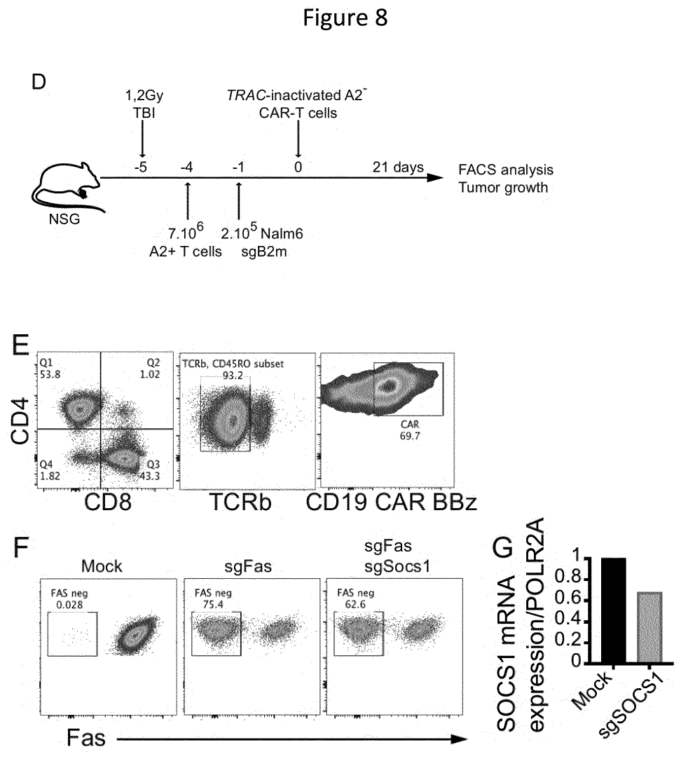

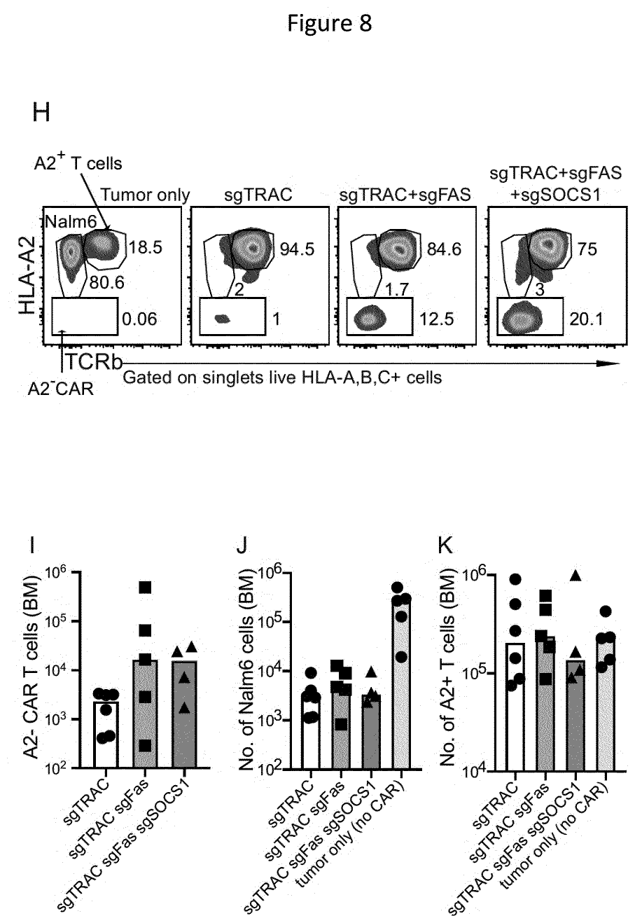

The inventors previously demonstrated that Ag-exp CD4+ transgenic T cell proliferation is inhibited during an ongoing immune response while naive T cells of the same specificity are able to proliferate efficiently (Helft et al., 2008). To unravel the inhibitory mechanisms controlling the proliferation of Ag-exp CD4⁺ T-cell, they used the A^{b}:Dby-specific Marilyn monoclonal CD4⁺ T cells (from the TCR-Tg Rag2^{-/-} Marilyn mouse (Lantz et al., 2000)). After intravenous (i.v.) adoptive transfer of naïve CD45.2 Marilyn CD4⁺ T cells into C57BL/6 hosts, they initiated an immune response by injecting Dby peptide-loaded dendritic cells (DCs) into the footpad (FIG. 1A). To track the fate of newly recruited Ag-specific CD4⁺ T cells into such an ongoing immune response, they let the first cohort of primed Marilyn

cells expand for a week before injecting i.v. a second cohort of naive or in vitro activated CD45.1 Marilyn CD4+ T cells (Ag-exp) (Helft et al., 2008). In this monoclonal recall response, in vitro primed Ag-exp CD45.1 Marilyn CD4+ T cells exhibit a reduced proliferation and capacity to produce IL-2 as compared to naive CD45.1 Marilyn T cells (FIG. 1B-D). This model opens up the possibility to genetically manipulate Ag-exp CD4+ T cells before analyzing their fate in vivo during an immune response.

To identify the intrinsic negative regulators of the CD4+ T cell immune response, they performed a positive genome-wide CRISPR screen looking for genes whose inactivation would restore the proliferation of Ag-exp CD4+ T cells during an immune response. They transduced in vitro-generated Ag-exp Marilyn-R26-Cas9 (Cas9) T cells with a genome-wide knockout (GWKO) sgRNA lentiviral library (18400 genes, 90K sgRNA)(Tzelepis et al., 2016), achieving 20-25% efficiency (BFP+)

114 (FIG. 1E). After puromycin selection, 40% of the transduced T cells survived, revealing a 75% single infection rate (Chen et al., 2015). Prior injection into the adoptive hosts, mock and library transduced Ag-exp Marilyn-Cas9 T cells exhibited a central memory phenotype (CD62L+CD44+), allowing them to similarly home to the dLN (FIG. 1E). Analysis of sgRNA in the transduced MarilynCas9 T cells revealed that less than 0.5% of the sgRNA were under-represented as compared to the original plasmid library (FIG. 1F). They conducted two independent GWKO pooled screens with 12×10⁶ Ag-exp library-transduced or mock-transduced Marilyn-Cas9 T cells per C57BL/6 mouse (FIG. 1A). Seven days after transfer and priming, mock-transduced Marilyn-Cas9 cells proliferation was abolished. However, the proliferation of the library-transduced Marilyn-Cas9 cells was significantly restored, as shown by the higher ratio of BFP⁺/BFP⁻ in the CFSE^{lo} subset compared to mock-transduced Marilyn-Cas9 cells ratio, indicating the release of the proliferative blockade by some sgRNA (FIG. 1G).

In the absence of the first cohort, library-transduced Marilyn cells expanded to some extent, attesting for an efficient priming (FIG. 1G). After CFSE-based cell sorting of CD45.1 Marilyn-Cas9 T cells, amplified sgRNA sequences enriched in the CFSE^{lo} subset were compared to sgRNA from non-dividing T cells (CFSE^{hi}). The small fraction of sgRNA represented in the CFSE^{lo} subset attest the effectiveness of the in vivo selection. Analysis of individual sgRNA enriched in the CFSE^{lo} subset from two independents screens identified Socs1 as the major gene involved in the restored proliferation of Ag-exp CD4+ T cells in vivo (p<1·10⁻⁶, false discovery rate (FDR)<1%) (FIG. 1H), while other lower ranking targets presented a FDR>0.5. Interestingly, Socs1 sgRNA were also significantly enriched in the CFSE^{lo} subset of library-transduced Marilyn cells injected alone compared to the CFSE^{hi} subset, consistent with the capacity of Ag-exp CD4⁺ T cells to inhibit one another. Altogether these data support a non-redundant and critical role for SOCS1 in T cell biology, in particular for CD4⁺ T cells that had not yet been explored.

The inventors next assessed the impact of SOCS1 inactivation on Ag-exp CD4⁺ T-cell proliferation using electroporation of individual sgRNA Cas9 ribonucleoprotein complexes (RNPs) (Seki and Rutz, 2018) in two different CD4+ TCR-Tg models, Marilyn, and OT2 cells (the latter expresses a TCR specific for MHC-II restricted ovalbumin peptide).

Briefly, in vitro primed CD4+ TCR-Tg cells were electroporated with RNPs. 2·10⁶ naive, Ag-exp mock or Ag-exp sgSOCS1 CD4⁺ T cells were CFSE142-labeled and subsequently injected as secondary responders into C57BL/6 mice during an ongoing immune response. In both models, the large naive CD4+ T cell expansion indicated efficient priming whereas Socs1 gene inactivation unleashed the brake observed in mock Ag-exp CD4⁺ T cells proliferation (FIG. 1I). These results uncover a role for SOCS1 as a major intrinsic regulator responsible for Ag-exp CD4+ T cell arrest during an ongoing immune response. Notably, the inventors did not observe any Treg conversion after Marilyn and OT2 cells transfer in vivo, suggesting that Ag-specific Tregs are not involved in their models, contrary to what was suggested in another report (Akkaya et al., 2019).

### SOCS1 is a Critical Node Integrating Multiple Cytokine Signals to Actively Inhibit CD4⁺ T Cell Functions

To mechanistically characterize SOCS1-mediated inhibition of CD4⁺ T cells, the inventors sought for potential inducers and subsequently assessed the functional consequences of Socs1 inactivation on Ag-exp CD4⁺ T cells. SOCS1 expression in murine splenocytes is induced by both cytokines and TCR stimulation with different timelines and intensities (Sukka-Ganesh and Larkin, 2016). Although basal levels of SOCS1 are present in untreated T cells, increase in SOCS1 protein level in response to cytokine stimulation arises rapidly (6 hours) while its maximal expression occurs 48h after TCR stimulation (Sukka-Ganesh and Larkin, 2016). This is in accordance with the timeframe of inhibition in their model, which starts in vivo 2 days after priming (Helft et al., 2008). These results suggest that TCR engagement in the presence of cytokines could be the reason for SOCS1 induction in Ag-exp CD4⁺ T cells.

To assess if a differential sensitivity to cytokine signaling could explain the selective inhibitory activity between naive and antigen experienced cells, the inventors compared the transcriptional expression of cytokine receptors between sorted proliferating (*) and inhibited subsets (**) during an ongoing immune response (FIG. 2A). They observed a significantly increased expression of Il2ra (also called CD25, confirmed at protein level), Ifngr1 and Ifngr2 in the CFSE^{hi} cells as compared to CFSE^{lo} cells (FIG. 2B) This correlation between inhibited cells and expression of cytokines receptors was confirmed at the protein level. Moreover, naïve and Ag-exp CD4⁺ T cells secreted IL-2, while only Ag-exp Marilyn CD4⁺ T cells produced both IL-2 and IFN-γ.

As SOCS1 is a known regulator of IFN-γ signaling (Alexander et al., 1999), they evaluated the proliferation of Ag-exp IFN-γR^{-/-} Marilyn cells during an ongoing immune response, but the absence of the receptor marginally restored the expansion of these cells in vivo (FIG. 2C). SOCS1 can also be induced by IL-2 in T cells and associates with IL-2RP (Liau et al., 2018) to potently inhibit IL-2-induced Stat5 function (Sporri et al., 2001). Using blocking antibodies concomitant with Ag re-stimulation of Ag-exp CD4⁺ T cells in vivo, the inventors then assessed the roles of IL-2 and IFN-γ alone and in combination in this inhibition. The blockade of IL-2 signaling using anti-mouse IL-2Rb, which inhibits binding of IL-2 to the IL-2R did not reverse Ag-exp CD4⁺ T cells impaired proliferation (FIG. 2D). However, blockade of both IL-2 and IFN-γ signaling (using anti-IFN-γRa and Ag-exp IFNγ-R^{-/-} Marilyn T) significantly rescued the expansion of re-stimulated Ag-exp Marilyn T cells (FIG. 2D).

This shows a redundancy between the two cytokine receptors upstream of SOCS1 to impair Ag-exp CD4⁺ T cells expansion.

Then, the inventors estimated the functional consequence of Socs1 deletion on Ag-exp CD4⁺ T cells TCR induced activation, reflected by expression of the early activation marker CD69, the late activation marker CD25 and the T cell receptor responsive transcription factor Interferon Regulatory Factor 4 (IRF4) (FIG. 2E). After overnight stimulation with titrated peptide-pulsed DCs, both Ag-exp Socs1-inactivated Marilyn and OT2 cells displayed similar sensitivity (Ag dose leading to 50% of the maximum response) to Ag stimulation as compared to mock-treated cells. However, the inventors observed a striking increase in CD25 and IRF4 expressions at higher Ag doses with an elevated “plateau” (FIG. 2E). This suggests that SOCS1 does not directly regulate proximal signals induced by cognate peptide stimulation but rather inhibits downstream signaling events This would suggest the release of a negative feedback loop, related to the secretion of IL-2 and IFN-γ in the medium.

As IRF4 is the central regulator of Th1 cytokines secretion in CD4⁺ T cells (Mahnke et al., 2016; Wu et al., 2017), they evaluated the capacity of Socs1 inactivated CD4⁺ T cells to display polyfunctionality. Socs1 inactivated Marilyn and OT2 cells exhibited higher percentage of Th1 polycytokine (IFN-γ-, TNFα- and IL-2-) production after re-stimulation (FIG. 2F). Thus, by integrating several cytokine signals, SOCS1 actively hampers polyfunctionality of Ag-exp CD4⁺ T cells.

These findings show that SOCS1 is a node capable of receiving signals from several inputs (IFN-γ and IL2) to abrogate multiple signaling outputs, leading to blockade of proliferative and effector functions.

### Socs1-Inactivation in Tumor-Reactive Marilyn CD4⁺ T Cells Induces a Polyfunctional Cytotoxic Phenotype Enhancing the Rejection of Male Bladder MB49 Tumors

The restored function of Socs1-inactivated Ag-experienced CD4⁺ T cells led the inventors to evaluate the therapeutic potential of Socs1 deletion on adoptively transferred antitumor CD4⁺ T cells. Inventors challenged female C57BL/6 mice with the Dby (HY)-expressing MB49 male bladder carcinoma cells and 10 days later intravenously transferred mock or sgSOCS1 Ag-exp Marilyn cells (FIG. 3A).

In the absence of Marilyn cell transfer, the immunogenic but nevertheless aggressive MB49 tumors grew unimpeded by the endogenous immune response (FIG. 3B, C). The transfer of mock Ag-exp Marilyn cells led to the CD8⁺ T cells- and NK cells-dependent rejection of MB49 tumors (FIG. 3B, C). However, the transfer of Ag-exp sgSOCS1 Marilyn CD4⁺ T cells induced a tumor rejection that was partially maintained after depletion with antibodies (FIG. 3B, C).

To determine whether Marilyn sgSOCS1 T cells were helpers or “stand-alone” effectors in ACT, the investigators analyzed the number, phenotype and transcriptome of the transferred Marilyn T cells in the tumor draining lymph nodes (TdLN), in the tumors and in a distant irrelevant LN (irr-LN) before tumor rejection, at day 7. Surprisingly, the inventors observed that Ag-exp sgSOCS1 Marilyn T cells infiltrate tumors more efficiently than mock Marilyn cells by nearly ten-fold (FIG. 3D). This was associated with a higher percentage of proliferating Ag-exp sgSOCS1 Marilyn cells in TdLN-infiltrating as compared to mock Marilyn cells, which displayed dominant arrest in their proliferation (FIG. 3E).

Mirroring this increased proliferation, bulk RNAseq analysis of Marilyn cells sorted from TdLN revealed upregulation of genes implicated in cell cycle and DNA replication (G2M checkpoints, E2F transcription factors, mitotic spindle) as well as IL2/STAT5 signaling in sgSOCS1 Marilyn cells (FIG. 3F). This pathway, together with molecules such as Il12rb2, Il2rb, Tbx21, Cxcr3, Cxcr5, Ifng and Ctla2b (FIG. 3G) have been recently implicated in the differentiation program of CD4 T helper-1 (Th1) cells with cytotoxic features (Krueger et al. 2021; Śledzińska et al. 2020b) and polyfunctional antitumor activity (Z.-C. Ding et al. 2020). Inspection of protein expression in Ag-exp sgSOCS1 Marilyn T cells confirmed an increased polyfunctionality, highlighted by the expression of Th1 cytokines in the TdLN and the capacity to produce Granzyme B at the tumor site (FIG. 3H, I).

Altogether, these data along with the strong expression of MHC-II molecules by MB49 tumors (FIG. 3J), suggest that in addition to their role as helper T cells, Ag-exp sgSOCS1 Marilyn T cells could directly be cytotoxic and tumoricidal.

Thus, Socs1 deletion enables Marilyn CD4 T cells to undergo robust expansion and persistence in vivo, infiltrate tumors as well as elicit antitumor responses with a polyfunctional molecular signature indicative of Th-cytotoxic features (FIG. 3B, C, D, G, H, I).

Differential Effect of Socs1-Inactivation on the Properties of CD4⁺ and CD8⁺ T Cells Used for Adoptive Transfer Against Melanoma Tumors

To compare the biological impact of Socs1 deletion in CD4+ and/or CD8⁺ T cells on anti-tumor response, the inventors independently generated in vitro activated tumor specific CD4+ and CD8⁺ T cells in which they deleted or not SOCS1 as described above (FIG. 4A). they used CD90.1 OT2 CD4+ and CD45.1 OT1 CD8⁺ T cells recognizing MHC-II and MHC-I restricted ovalbumin peptides, respectively and subcutaneously implanted B16-OVA melanoma cells as tumor model, without conditioning or cytokines supply (FIG. 4A). As compared to the results displayed in FIG. 3, the inactivation of Socs1 in OT2 cells had a marginal antitumor effect (FIG. 4B, C). This could be related either to the use of the highly immunosuppressive B16 melanoma model or to the co-transfer of large number of high avidity antitumor specific CD8⁺ T cells.

However, after adoptive transfer of sgSOCS1 OT1 T cells (FIG. 4B, C), the inventors observed a significant and durable rejection of established tumors as compared to transfer of mock OT1 T cells (p<0.001, log-rank). The infiltration of T cells seven days after transfer showed an increased accumulation in the TdLN and in the tumor for the group receiving both sgSOCS1 OT1 and sgSOCS1 OT2 cells as compared to mock transferred cells (FIG. 40).

Importantly, in the TdLN, Socs1 inactivation had a profound effect on OT2 CD4⁺ T-cell proliferation with a large increase in fully divided CD4⁺ T cells, whereas the pattern of OT1 CD8⁺ T-cell proliferation was barely affected, suggesting that SOCS1 impacts CD8⁺ T-cell survival more than proliferation (FIG. 4E). Sixty days after transfer, the number of sgSOCS1 OT2 cells ultimately decreased in the blood of B16-OVA challenged mice, while a population of central memory sgSOCS1 OT1 cells remained 15-fold more abundant than mock OT1 cells.

These results suggest that SOCS1 decreases the survival of Ag-exp CD8⁺ T cells or prevent the generation of long-lived subsets of CD8⁺ T cells. The former hypothesis is more likely, as tumor-infiltrating sgSOCS1 OT1 cells analyzed 14 days after transfer expressed higher mRNA levels of molecules involved in T cell survival (Tnfaip3, Bcl2, Il2ra, Il2rb, Jak2) and cytotoxic/effectors molecules (Gzmb, Ifngr, Irf1, FasI, Srgn, Tbx21). Moreover, hallmarks analysis highlighted pathways in tumor-infiltrating sgSOCS1 OT1 cells (FDR<0.05), associated with TNFα, IL-2 and IFN-γ responses. Interestingly, the GSEA of Socs1-inactivated OT1 T cells indicates that genes associated with effector functions are more expressed than those implicated in exhaustion.

Targeting Socs1 in both OT1 and OT2 cells preserved cytokine production associated with effector function in both CD4⁺ and CD8⁺ T cells (FIG. 4F, G), while GzmB was increased in CD8⁺ T cells (FIG. 4F). Overnight in vitro stimulation of sgSOCS1 OT1 cells with titrated SIINFEKL-pulsed DCs led to increased IFN-γ and granzyme B production at high antigen doses after Socs1 inactivation, showing that Socs1 actively restrains these cytokines in CD8⁺ T cells.

The preserved or increased functionality associated with the increased number of both sgSOCS1 OT2 and OT1 cells led to a much higher number of effector cells at the tumor site (FIG. 4F, G), likely explaining the stronger anti-tumor effect of Socs1 inactivated T cells. Altogether, these results show that SOCS1 has an intrinsic and differential role in the regulation of both CD4⁺ and CD8⁺ T cells in vivo.

### Immunotherapeutic Potential of SOCS1-Edited Human CD4+ and CD8+ CAR T Cells

To investigate the therapeutic potential of SOCS1 on human T-cell adoptive transfer, the inventors inactivated SOCS1 gene using Cas9 RNPs in human peripheral blood lymphocytes (PBL) that had been activated and then transduced with a chimeric antigen receptor, encompassing 4-1 BB co-stimulatory domains targeting CD19, referred to as 19BBz (FIG. 5A, B).

This construct, known to preferentially enhance the survival of CD8⁺ CAR-T cells (CAR8) (Guedan et al., 2018), allowed them to investigate the impact of SOCS1 inactivation on CD4+ CAR-T cells (CAR4), which have a limited in vivo life-span (Turtle et al., 2016; Yang et al., 2017b).

After overnight co-culture with the acute lymphoblastic leukaemia (ALL) FFLuc-BFP NALM6 cell line (NALM6), sgSOCS1 CAR4 and sgSOCS1 CAR8, produced higher levels of the effector molecules TNFα, IFN-γ and GzmB as compared to mock CAR T cells in three healthy donors, consistent with the 2-fold higher killing activity.

Furthermore, the inventors modelled CAR therapy in vivo by injecting 4·10⁶ PBL mock or sgSOCS1-treated (2·10⁶ CAR4 and 2·10⁶ CAR8 cells) in NALM6-infused NOD-scid IL2Rg^{-/-} (NSG) mice. Seven days after transfer, the number of sgSOCS1 CAR T cells accumulating in bone marrow (BM) was 2-fold higher than that of mock CAR T cells (FIG. 5C, D).

Reflecting the higher T cell infiltration in the bone-marrow and a more efficient tumor control, the transcriptomic profiles of sgSOCS1 CAR4 and CAR8 cells evidenced upregulation of molecules associated with activation (FOS, JUND, CD69, SOCS3), with long-lived associated factors (IL7R, P/M1 (Knudson et al., 2017), TCF7 (Zhou and Xue, 2012) and KLF2 (Carlson et al., 2006)), resistance to apoptosis (BCL2L11 (Hildeman et al., 2002) NDFIP2 (O'Leary et al., 2016)), key regulators of cytotoxic effector functions (GMZB, the interferon-induced molecules GBP5 (Krapp et al., 2016) and IRF1 and killer associated NKG7 (Patil et al., 2018)) (FIG. 5E).

As observed in several studies on CAR-T cell kinetics (Guedan et al., 2018) and CD4/8 CAR T subset analysis in ALL patients (Turtle et al., 2016; Yang et al., 2017b), CAR8 expanded preferentially over CAR4 in the inventor's model. They therefore examined the persistence of sgSOCS1 CAR T-cells, 28 days after transfer. Whereas mock CAR4 declined over time, sgSOCS1 CAR4 and sgSOCS1 CAR8 significantly accumulated in both BM and spleen of NSG mice, correlating with NALM6 rejection. Most strikingly, sgSOCS1 CAR4 expanded to the level of sgSOCS1 CAR8 (FIG. 5C, D). Accordingly, as compared to their mock CAR counterparts in the bone marrow, both sgSOCS1 CAR4 and CAR8 expressed increased levels of cytotoxic/effector-related molecules including IFNG, FCRL6 (Wilson et al., 2007), CTSB (Balaji et al., 2002), TBX21, as well as SOCS1-known targets/survival genes such as IL2RB, JAK3, BCL3 and CXCL13, consistent with the anti-tumor activity of sgSOCS1 CAR-T cells (Li et al., 2019).

Interestingly, the inventors observed a subset-specific transcriptomic pattern in SOCS1-inactivated CAR4 and CAR8 cells. On one hand, CAR4 harbored increased expression of genes associated with a proliferation signature represented by E2F targets (FIG. 5F) and genes involved in metabolism such as the insulin growth factor regulator HTRA1 (H. Ding et Wu 2018) and the AMPK-TORC1 metabolic checkpoint NUAK1 (Monteverde et al. 2018). On the other hand, CAR8 displayed signs of enhanced cytotoxicity (GZMB, GZMH, TNFSF10 (TRAIL), Secreted And Transmembrane 1 SECTM1 (T. Wang et al. 2012), Killer Cell Lectin Like Receptor D1 KLRD1 (H. Li et al. 2019)), some of which were confirmed by flow cytometry analysis (FIG. 5G, H).

Furthermore, contrary to sgSOCS1 CAR4 cells, sgSOCS1 CAR8 in vivo expressed lower levels of E2F targets (FIG. 5F) and downregulated genes involved in cell cycle and DNA replication, suggesting that the higher number of cells found in the BM is more related to survival than proliferation (Ren et al. 2002).

While sgSOCS1 CAR cells exhibited a PD1⁺LAG3⁺ phenotype, suggesting an increased level of activation, the transcriptional signature of sgSOCS1 CAR8 at day 28 and overtime (day 28-day7) was more similar to an effector memory than an exhausted phenotype (Wherry et Kurachi 2015).

For sgSOCS1 CAR4, the GSEA analysis did not reveal a significant exhaustion phenotype, and except for PRDM1 (Blimp1) no specific transcription factors, including BATF, TOX, EOMES or BCL6 were upregulated as compared to mock CAR4 cells at day 28 (Fig. S5J). Altogether, this indicates that even if SOCS1 inhibition leads to hyperactivation in CAR T cells, without evidence of exhaustion.

At this late time point, SOCS1 inactivation not only led to increased numbers of CAR4 and CAR8 but also to higher cytokine secretion and cytotoxic activity (FIG. 5G, H).

To unravel the relative contribution of CAR4 versus CAR8 T cells to tumor rejection and the importance of SOCS1-inactivation in each subset, the inventors tracked the bioluminescence of NALM6 tumors in vivo treated with the following combinations: mock CAR4 mock CAR8, sgSOCS1 CAR4 sgSOCS1 CAR8, mock CAR4 sgSOCS1 CAR8, sgSOCS1 CAR4 mock CAR8.

Although we saw a significant delay of tumor progression in CAR4 mock CAR8 sgSOCS1 and CAR4 sgSOCS1 CAR8 mock groups, SOCS1 targeting in both subsets was essential to achieve tumor eradication (FIG. 5J, K). This suggests that both the robust expansion, persistence and functionality of sgSOCS1 CAR T cells account for their optimal synergistic antitumor effects.

Altogether, SOCS1 deletion in both CAR4 and CAR8 represent a major target to improve ACT therapeutic efficacy against solid and blood cancers.

### DISCUSSION

Looking for the mechanisms involved in the regulation of CD4⁺ T cell proliferation during an antigenic response, inventors uncovered SOCS1 as a non-redundant signaling node, leading to a negative feedback loop downstream of TCR and lymphokines signaling. SOCS1 appears to actively restrain T cell proliferation, survival and effector functions in vivo.

SOCS1 evidenced different inhibitory effects on CD4⁺ and CD8⁺ T cells: it can abrogate CD4⁺ T cell proliferation, survival and polyfunctionality, while it mostly reduced CD8⁺ T cell survival and effector function. The present data further demonstrate a potent effect of Socs1 gene inactivation on CD4 T cell expansion, which is of particular relevance for improved CAR-T cells composition and efficacy.

In the case of synchronous immune responses, which can be induced by systemic infection or I.V injection of antigen, all naive CD4 T cells are simultaneously recruited. However, during a localized asynchronous immune response, new naive cells and recirculating Ag-experienced CD4 T cells keep entering into the LN.

The cohort system enables to distinguish between naïve and Ag-exp CD4 T cells during an ongoing immune response and evaluate their intrinsic differences. Our previous data (Helft et al. 2008) and the current work show that when both naïve and Ag-experienced CD4 T cells are present, as it often happens during a recall response, the Ag-exp CD4 T cells are at disadvantage with regards to proliferation.

This strong and very reproducible inhibition of Ag-exp CD4 T cells, probably responsible for CD4 T cell response diversity and polyclonality, is not related to the disappearance of Ag, the suppression by regulatory T cells or the competition among responding T cells for APCs.

Instead, the study supported the existence of direct T-T interactions, which lead to the intrinsic, dominant and preferential inhibition of effector/memory CD4 T cells proliferation (Helft et al. 2008). This is also true with solid tumor injected S.C, where Ag-exp CD4 T cells interact one another in the TdLN, sparing the newly arriving naive CD4 T cells.

The inventors have now demonstrated that this inhibition is due to the TCR-induced expression of SOCS1 and cytokines receptors. Surprisingly, their in vivo genome-wide positive screen only evidenced Socs1, most likely because the genes necessary for in vitro growth and survival were missed as the inventors used a constitutive CRISPR/Cas9 system.

In addition, the restored proliferation of Ag-exp Marilyn cells by blocking both IL2 and IFN-γ pathways in their model (FIG. 2D), suggests a genetic redundancy and compensation between inactivated receptors that could not be revealed by their screening strategy. Although not demonstrated in the current work, the inventors suspected that, during the synaptic T-T interaction, the Ag-exp CD4 T cells (starting two days after TCR triggering), expressing high levels of SOCS1 and cytokine receptors, are inhibited by IL2/IFN-γ produced in “cis” and by IL2 produced by naïve cells in “trans”, which activates SOCS1.

Finally, the investigators demonstrated that SOCS1 is a major intrinsic inhibitor of Ag-exp CD4⁺ T-cell expansion in vivo in two different CD4⁺ T-cell models (Marilyn and OT2) exhibiting distinct avidities and using various types of antigenic stimulation such as DC-peptide or tumor challenge.

Altogether, this highlights the generalizable aspect of the present invention, operating for all CD4⁺ T cells.

The inventor's data suggest that cytokine sensing plays a role in impairing CD4⁺ T cells immunity after Ag re-exposure/chronic stimulation.

This paradoxical cytokine-mediated suppression of CD4⁺ T cells has already been described, when blocking chronic IFN-I signaling during persistent infection enhanced CD4⁺ T cell-dependent virus clearance (Teijaro et al. 2013; Wilson et al. 2013).

SOCS1 may be responsible for the so-called activation induced cell death (AICD), where IL-2 (Lenardo 1991) or IFN-γ (Berner et al. 2007) provided too early after antigen stimulation leads to apoptosis of CD4⁺ T cells (Majri et al. 2018).

Hence, the authors observed that SOCS1 prevented the expression of genes involved in resistance to apoptosis, such as Bcl2, Bcl3, Tnfaip3, Hopx (Albrecht et al. 2010).

SOCS1 also appears to selectively regulate the proliferation of CD4⁺ T cells as compared to CD8⁺ T cells in vivo by inhibiting the expression of E2F targets, key regulators of cell cycle progression, in both human and murine CD4⁺ T cells (J. W. Zhu et al. 2001).

Thus, targeting SOCS1 improves CD4⁺ T cells survival and proliferation by rendering them insensitive to out of sequence lymphokine-induced cell death. This phenomenon has been described for SOCS3, another member of SOCS family, which is involved in the impairment of human and murine CD4⁺ T cells in vivo, after cytokine pre-exposure (Sckisel et al. 2015). However, SOCS3 expression is associated with Th2 lineage commitment, while SOCS1 is involved in Th1 differentiation (Egwuagu et al. 2002).

As SOCS1 negatively regulates Ag-exp CD4⁺ T-cell capacity to produce several cytokines essential for anti-tumor immunity (Dobrzanski 2013) in vitro (FIG. 2), inventors explored the impact of Socs1 deletion on adoptively transferred antitumor CD4⁺ T cells. Targeting SOCS1 also increases Ag-exp CD4⁺ T-cell polyfunctionality in vivo, enhancing their lymphokines secretion, in particular IFN-γ in the TdLN (FIG. 3) and GZMB at the tumor site (FIG. 3, FIG. 5).

Thus, both murine and human CD4⁺ T cells targeted for SOCS1 exhibit an increased expression of Th1 phenotype with cytotoxic features at the tumor site (FIG. 3, FIG. 5).

The acquisition of such polyfunctional features by CD4⁺ T cells have been recently described as a two-steps modular program involving IL2/STAT5/BLIMP1 (Śledzińska et al. 2020b), and IFN-γ/IL12/ZEB2 (Krueger et al. 2021).

These molecules were significantly upregulated in sgSOCS1 CD4⁺ T cells and the constitutive activation of STAT5 is essential to drive polyfunctional antitumor activity (Z.-C. Ding et al. 2020).

This show that deleting SOCS1 participate in the induction of such differentiation program, which improves adoptive CD4⁺ T-cell antitumor immune response.

With no effect on OT1 CD8 T cell CFSE pattern (FIG. 4E), downregulation of KEGG pathways associated with cell cycle/DNA replication in sgSOCS1 CAR8 at day 7 and E2F targets/G2M checkpoint genes at day 28 (FIG. 5F), it seems that SOCS1 inactivation rather reduces the Ag-dependent proliferation of CD8 T cell in vivo.

Accordingly, a defective expansion following Ag stimulation has been reported before in SOCS1-deficient CD8⁺ T cells in vivo (Ramanathan et al. 2010).

However, SOCS1 targeting can still potentiate the cytokine-driven (Ag-independent) proliferation of CD8 T cell in vitro (Ramanathan et al. 2010; Shifrut et al. 2018), promote the survival of CD8 T cell (FIG. 5E) accumulating at the tumor site (FIG. 4D) and robustly increase their cytolytic activity (FIG. 4F, FIG. 5G) (Shifrut et al. 2018; Wei et al. 2019; Zhou et al. 2014)) in a TCR-dependent manner.

Finally, the inventors demonstrate both in TCR-Tg and CAR CD8 T cell, that SOCS1 inactivation induce a differentiation in effector-memory phenotype with no significant sign of exhaustion.

With an improved persistence in vivo, SOCS1 targeted CD4⁺ T cells are probably subjected to chronic stimulation that might lead to anergy and Treg conversion (Alonso et al. 2018).

However, SOCS1 is essential for the maintenance of Foxp3 expression and for Tregs suppressive functions in vivo (Takahashi et al. 2011; 2017).

Accordingly, Socs1-inactivated CD4⁺ T cells display enrichment of conventional T cells markers as opposed to Tregs genes as well as a decreased gene expression of FOXP3 and IKZF2 in sgSOCS1 CAR4 as compared to mock CAR4 at late time point.

Altogether, the authors confirmed that targeting SOCS1 in CD4⁺ T cells prevent them from converting into Tregs.

The forced expression of cytokine-encoding genes or construct containing a JAK/STAT signaling domain in CD8⁺ CAR-T cells improves their persistence and antitumor effects in vivo, highlighting the importance of signal 3 (mediated by cytokines and initiated after CD3 signaling: signal 1 and co-stimulation: signal 2) for CAR-T cell functions (Markley et Sadelain 2010; Quintarelli et al. 2007; Kagoya et al. 2018).

Here, the inventors demonstrate that inactivating a major inhibitor of cytokines signaling in CAR-T cells also enhance their therapeutic potential and most importantly selectively affect CD4+ and CD8+ CAR-T cells.

This has major relevance for design of the next generation of adoptive T cells therapies for cancer and viral infections with improved efficacy and optimized CD4/CD8 composition.

The inventors unravel the importance of signal three regulation in CD4⁺ T cell biological functions and identified a major intracellular checkpoint critical for the magnitude, duration and quality of T cell immune responses, that may prove efficacy in clinics.

2) In Vivo Genome-Wide CRISPR Screens Identify Targets for Escaping Host Immune Rejection

### In Vivo Genome-Scale (18400 Genes) CRISPR Pooled Screens Identify Fas and B2m as Non-Redundant Targets Allowing T Cell Survival in MHC-Mismatched Hosts

To answer major clinical needs for the design of next generation ATCT¹, the inventors have developed genome-scale (GS) CRISPR screens to identify factors allowing T cells resistance to allogeneic cell death.

The inventors set up the screening conditions of allogeneic rejection by transferring activated Marilyn CD4 T cells (C57BL6, H2-Kb) into the fully MHC-mismatched BALB/c mice (H2-Kd) and demonstrated that already 4 days post injection, most of the donor T cells were rejected from the spleen (FIG. 6A, B), allowing them to perform the screen with a targeted window.

To overcome the delivery challenges of Cas9 in primary T cells, the inventors have first crossed Rosa26-Cas9 knock-in mice (Cas9 widespread expression and eGFP) (41), with CD45.1/1 Marilyn (CD4) anti-Dby TCR-transgenic (42) TCR-transgenic Rag2^{-/-} mice.

The genome-wide genetic inactivation of T cells was achieved through incorporation of specific single guide RNA (sgRNA), using the Mouse Improved Genome-wide Knockout CRISPR lentiviral Library v2 (Addgene #67988, BFP reporter) consisting of 90 230 sgRNA targeting 18,400 murine genes (FIG. 6C).

This innovative approach allows rapid, systematic and unbiased identification of T-cell intrinsic limiting factors, functionally non-redundant in vivo (Dong et al. 2019; Wei et al. 2019).

By transferring 10⁷ mock or library-mutated CD45.1 Marilyn T cells (C57BL6, H2-Kb) into the fully MHC-mismatched BALB/c mice (H-2K^{d}), the inventors have demonstrated that library-mutated Marilyn T cells could survive significantly better than Mock Marilyn T cells in BALB/c mice (FIG. 6 D, E). Surviving library-mutated Marilyn T cells were sorted from harvested spleens at day 4 and gDNA analyzed by deep sequencing.

The MAGECK analysis (W. Li et al. 2015) of sgRNA enriched in the gDNA of library-mutated CD45.1 Marilyn T cells from BALB/c mice compared to the diversity of sgRNA from C57BL6 mice highlighted Fas and β2m (p<10⁻⁶, FDR<0.07) as potential targets for reduced allogeneic rejection of T cells (FIG. 6F, G).

For validations experiments, the inventors used Marilyn T cells expressing different congenic markers allowing to precisely control the survival of Fas or B2m-inactivated T cells (CD45.1/1) as compared to Mock Marilyn T cells (CD45.1/2) in each mouse.

Switching to a Cas9-ribonucleoprotein (RNP) strategy (Doench et al. 2016) to efficiently inactivate either Fas or B2m gene in expanded-Marilyn T cells (60-70% inactivation, data not shown), the authors demonstrated a significantly improved survival of Fas-inactivated T cells (H2-Kb) in BALB/c mice (H2-Kd) at day 4 (FIG. 6H, I). Similarly, B2m-inactivated Marilyn T cells were surviving better in BALB/c mice as compared to Mock Marilyn T cells, which benchmark the success and technical rigor of our screening strategy (FIG. 6J, K).

### Fas Targeting Improves Resistance to Both CD8 T-Cell and NK Cells-Mediated Allogeneic Rejection and can be Potentiated In Vivo by Socs1-Inactivation

Cellular immune rejection is known to be mediated by activated host alloreactive T and NK cells (Elliott et Eisen 1988; Ciccone et al. 1992; Ruggeri et al. 2002).

In the inventors first model (C57BL6 T cells in BALB/c mice), B2m-inactivated and H2-Kb expressing Marilyn T cells were effectively eliminated from C57BL6 mice, while they remained alive in BALB/c mice at day 4 (FIG. 6J, K).

B2m-inactivation in T cells leads to the downregulation of MHC-1 molecules, which normally triggers their destruction by missing-self reactivity of NK cells (Bix et al. 1991). If this mechanism is efficient in C57BL6 mice, the host cellular rejection mediated by BALB/c mice seems however mostly mediated by alloreactive T cells.

To unravel the contribution of each subset in target-inactivated CD45.1 polyclonal T cells (CD4 and CD8) resistance to allogeneic rejection in BALB/c immunocompetent mice, the inventors used either NK cells- or CD8 T cells-depleting antibodies (anti-CD8a 2.43; anti-asialoGM1) concomitant to the in vivo selection process (FIG. 7A).

After depleting NK cells from BALB/c mice using anti-GM1 antibodies, the inventors did not observe any significant difference in CD45. 1 T cells (H2-Kb) splenic infiltration as compared to BALB/c+IgG mice (FIG. 7B, C).

However, the depletion of CD8 T cells using anti-CD8a antibodies was sufficient to restore mock CD45.1 T cells survival to the level of Fas-inactivated CD45.1 T cells (FIG. 7B, C).

Altogether, this demonstrated that the enhanced survival of Fas-inactivated H2-Kb T cells in BALB/c mice is due to resistance to alloreactive CD8 T cells lysis.

The inventors further hypothesized that the inactivation of Fas gene can prevent rejections by NK cells and allo-T cells as well as CAR T-cell fratricide.

Indeed, it has been recently shown that CAR-T cells can actively transfer their targeted antigen through trogocytosis, thereby promoting fratricide T cell (Hamieh et al. 2019a).

In addition to its persistence, a key aspect to the clinical success of allogeneic CAR T-cell therapy is its powerful and immediate antitumor response during the engraftment window.

The inventors have previously discovered that deletion of SOCS1, a non-redundant inhibitory checkpoint of activated T cells, could improve CAR-T cells expansion and effector functions (Del Galy et al. 2021)(FIG. 1-5).

Moreover, SOCS1-deleted CAR T cells upregulate TRAIL and FasL molecules (FIG. 4, 5), which are known escape mechanisms used by fetal trophoblast cells for maternal immune tolerance (Vacchio et Hodes 2005).

Therefore, the inventors hypothesized that SOCS1 and FAS dual inactivation would allow allogeneic CAR T-cell to robustly accumulate, be more functional and insensitive to fratricide (Hamieh et al. 2019b), in a weaponized graft advantaging system resembling the immune-privileged sites of human body (Forrester et al. 2008).

Finally, targeting SOCS1, which is a potent JAK/STAT inhibitor could also increase the cytokine-dependent proliferation and survival of TRAC-inactivated CAR T cells prior to infusion.

As shown FIG. 7D, E, the inventors have efficiently inactivated both Fas and Socs1 genes in polyclonal T cells from C57BL6 donor mice, expressing the congenic marker CD45.1. Four days after IV injection of Mock or targets-inactivated CD45.1 T cells, splenic infiltration revealed a significantly higher number of live Fas/Socs1-inactivated T cells as compared to Fas-targeted T cells in BALB/c mice (FIG. 7G). The fold change analysis demonstrated that Fas-inactivated T cells survived 10 times better than mock T cells and Fas/Socs1 dual inactivation induced a 30 times enhanced survival of allogeneic T cells in BALB/c mice (FIG. 7H).

The inventors designed a model of semi-allogeneic transfer in vivo, based on the injection F1 T cells (H2^{b}/d) generated by crossing either OTI (H2^{b}) or Marilyn (H2^{b}) mice with BALB/c H2^{d} mice (FIG. 7I).

In this model, the depletion of either NK cells (with anti-NK1.1 antibodies) or CD8 T cells (anti-CD8a 2.43) increased the survival of Mock F1 T cells, suggesting that both subsets are alloreactive in this semi-allogeneic transfer in C57BL6 recipients (FIG. 7J). Moreover, the inventors observed a higher percentage of surviving H2-Kd expressing F1 T cells after NK depletion as compared to H2-Kb live T cells after in vivo selection, implying a role of NK cells in the specific targeting of H2-Kd F1 T cells (FIG. 7K).

Similar to the previous model, the inventors demonstrated that Fas-inactivated F1 T cells survived better than mock F1 T cells in C57BL6 recipients and that Socs1 targeting can potentiate Fas-deleted T cells resistance to allogeneic destruction in vivo (FIG. 7 L, M).

Fas and SOCS1 Dual Inactivation Protects Murine and Human Tumor-Reactive T Cells from Alloimmune Cellular Rejection In Vivo

To assess the antitumor efficacy of the identified hits across major histocompatibility barriers, the inventors have adapted a previously developed protocol based on the transfer of TCR-Tg donor T cells from F1 mice (H2^{b/d}) into total body irradiated (TBI) and reconstituted C57BL6 recipient mice bearing a specific tumor (Boni et al. 2008).

This type of haploidentical transfer with conditioning regimen is closer to clinical settings that our in vivo screening strategy in fully mismatched hosts.

More importantly, it offers the possibility to evaluate the long-term behavior and extrinsic effects of synergistic targets in an immune-competent model, which is complementary to the functional validation with human CAR-T cells in NSG mice.

After crossing OTI (H2-Kb) or Marilyn (H2-Kb) mice with BALB/c H2-Kd mice, the F1 generation T cells (H2b/d) will have the ability to persist for up to 24 days in reconstituted C57BL6 (H2^{b}) 7 Gy-irradiated mice (FIG. 8A) and respectively control the growth of B16-OVA melanoma or male Dby-expressing bladder tumors MB49.

The F1 OT-1 T cells (H2^{b/d}) were efficiently inactivated for Socs1/Fas and injected into irradiated and reconstituted C57BL6 recipient mice bearing a B16-OVA tumor. At day 15, the inventors observed that adoptively transferred allogeneic tumor-specific sgFas/sgSocs1 T-cells persisted 10 times more in the spleen (FIG. 8B) and infiltrated 100 times more the tumor than their mock counterpart, while maintaining a cytotoxic capacity (FIG. 8C).

Adapted from an available protocol (Mo et al. 2020), the inventors have evaluated the function of Fas and Fas/SOCS1-inactivated CAR T-cell in an acute lymphoblastic leukemia model (human ALL, NAML6-luciferase cells), in which CAR-T cells must resist immune rejection from allogeneic T cells while protecting NOD/SCID/IL2rγnull (NSG) mice against cancer progression (FIG. 8D).

Briefly, NSG mice will receive a pretreatment cytoablation (TBI), which promotes a robust expansion of recipients T cells (A2⁺ T cells). Then, to avoid non-specific allogeneic rejection by A2⁺ T cells, HLA expression will be deleted from tumor cells (B2M-inactivation in NALM6-luciferase cells, Naml6 sgB2m) and donor CAR-T cells (A2-) will be TCR-inactivated to prevent the destruction of A2+ T cells.

After efficiently transducing CD4 and CD8 T cells from healthy donor with the CD19 CAR bbz construct (FIG. 8E), cells were subjected to TRAC, FAS and SOCS1-inactivations (FIG. 8E, F, G), after electroporation with sgRNA and HIFI Cas9.

The bone marrow infiltration of NSG mice 15 days after A2-CAR-T cells injections, revealed engraftment and persistence of A2+ T cells, leukemia eradication in all the groups treated with CAR-T cells and an increased survival of FAS-inactivated as well as FAS/SOCS1-inactivated A2-CAR T cells (FIG. 8H, I, J, K).

This data show that both FAS and FAS/SOCS1 targeting enhance the persistence of CAR-T cells in the presence of allogeneic recipient T cells, while retaining antitumor activity.

### REFERENCES

Albrecht, Inka, Uwe Niesner, Marko Janke, Astrid Menning, Christoph Loddenkemper, Anja A. Kuhl, Inga Lepenies, et al. 2010. «Persistence of Effector Memory Th1 Cells Is Regulated by Hopx». European Journal of Immunology 40 (11): 2993-3006. https://doi.org/10.1002/eji.201040936.
Alonso, Ruby, Heloise Flament, Sébastien Lemoine, Christine Sedlik, Emanuel Bottasso, Isabel Péguillet, Virginie Prémel, et al. 2018. «Induction of Anergic or Regulatory Tumor-Specific CD4+ T Cells in the Tumor-Draining Lymph Node». Nature Communications 9 (1): 2113. https://doi.org/10.1038/s41467-018-04524-x.
Bern, Michael D., Bijal A. Parikh, Liping Yang, Diana L. Beckman, Jennifer Poursine-Laurent, et Wayne M. Yokoyama. 2019. «Inducible Down-Regulation of MHC Class I Results in Natural Killer Cell Tolerance». The Journal of Experimental Medicine 216 (1): 99-116. https://doi.org/10.1084/jem.20181076.
Berner, Vanessa, Haiyan Liu, Qing Zhou, Kory L. Alderson, Kai Sun, Jonathan M. Weiss, Timothy C. Back, et al. 2007. «IFN-Gamma Mediates CD4+ T-Cell Loss and Impairs Secondary Antitumor Responses after Successful Initial Immunotherapy». Nature Medicine 13 (3): 354-60. https://doi.org/10.1038/nm1554.
Bix, M., N. S. Liao, M. Zijlstra, J. Loring, R. Jaenisch, et D. Raulet. 1991. «Rejection of Class I MHC-Deficient Haemopoietic Cells by Irradiated MHC-Matched Mice». Nature 349 (6307): 329-31. https://doi.org/10.1038/349329a0.
Boni, Andrea, Pawel Muranski, Lydie Cassard, Claudia Wrzesinski, Chrystal M. Paulos, Douglas C. Palmer, Luca Gattinoni, et al. 2008. «Adoptive Transfer of Allogeneic Tumor-Specific T Cells Mediates Effective Regression of Large Tumors across Major Histocompatibility Barriers». Blood 112 (12): 4746-54. https://doi.org/10.1182/blood-2008-07-169797.
Borst, Jannie, Tomasz Ahrends, Nikolina Bqbata, Cornelis J. M. Melief, et Wolfgang Kastenmüller. 2018. «CD4+ T Cell Help in Cancer Immunology and Immunotherapy». Nature Reviews. Immunology 18 (10): 635-47. https://doi.org/10.1038/s41577-018-0044-0.
Bos, Rinke, et Linda A. Sherman. 2010. «CD4+ T-Cell Help in the Tumor Milieu Is Required for Recruitment and Cytolytic Function of CD8+T Lymphocytes». Cancer Research 70 (21): 8368-77. https://doi.org/10.1158/0008-5472.CAN-10-1322.
Braud, V. M., D. S. Allan, C. A. O'Callaghan, K. Söderström, A. D'Andrea, G. S. Ogg, S. Lazetic, et al. 1998. «HLA-E Binds to Natural Killer Cell Receptors CD94/NKG2A, B and C». Nature 391 (6669): 795-99. https://doi.org/10.1038/35869.
Chakrabarti, S., G. Hale, et H. Waldmann. 2004. «Alemtuzumab (Campath-1H) in Allogeneic Stem Cell Transplantation: Where Do We Go from Here?″ Transplantation Proceedings 36 (5): 1225-27. https://doi.org/10.1016/j.transproceed.2004.05.067.
Champlin, R. E., J. R. Passweg, M. J. Zhang, P. A. Rowlings, C. J. Pelz, K. A. Atkinson, A. J. Barrett, et al. 2000. «T-Cell Depletion of Bone Marrow Transplants for Leukemia from Donors Other than HLA-Identical Siblings: Advantage of T-Cell Antibodies with Narrow Specificities». Blood 95 (12): 3996-4003.
Ciccone, E., D. Pende, O. Viale, C. Di Donato, G. Tripodi, A. M. Orengo, J. Guardiola, A. Moretta, et L. Moretta. 1992. «Evidence of a Natural Killer (NK) Cell Repertoire for (Allo) Antigen Recognition: Definition of Five Distinct NK-Determined Allospecificities in Humans». The Journal of Experimental Medicine 175 (3): 709-18. https://doi.org/10.1084/jem.175.3.709.
Corthay, Alexandre, Dag K. Skovseth, Katrin U. Lundin, Egil Rosjo, Hilde Omholt, Peter O. Hofgaard, Guttorm Haraldsen, et Bjarne Bogen. 2005. «Primary Antitumor Immune Response Mediated by CD4+ T Cells». Immunity 22 (3): 371-83. https://doi.org/10.1016/j.immuni.2005.02.003.
Del Galy, Aurélien Sutra, Silvia Menegatti, Jaime Fuentealba, Laetitia Perrin, Francesca Lucibello, Julie Helft, Aurélie Darbois, et al. 2021. «In vivo genome-wide CRISPR screens identify SOCS1 as a major intrinsic checkpoint of CD4⁺ Th1 cell response». bioRxiv, janvier, 2021.04.12.439455. https://doi.org/10.1101/2021.04.12.439455.
Depil, S., P. Duchateau, S. A. Grupp, G. Mufti, et L. Poirot. 2020. «“Off-the-Shelf” Allogeneic CAR T Cells: Development and Challenges». Nature Reviews. Drug Discovery, janvier. https://doi.org/10.1038/s41573-019-0051-2.
Ding, Huihua, et Tianfu Wu. 2018. «Insulin-Like Growth Factor Binding Proteins in Autoimmune Diseases». Frontiers in Endocrinology 9: 499. https://doi.org/10.3389/fendo.2018.00499.
Ding, Zhi-Chun, Huidong Shi, Nada S. Aboelella, Kateryna Fesenkova, Eun-Jeong Park, Zhuoqi Liu, Lirong Pei, et al. 2020. «Persistent STAT5 Activation Reprograms the Epigenetic Landscape in CD4⁺ T Cells to Drive Polyfunctionality and Antitumor Immunity». Science Immunology 5 (52). https://doi.org/10.1126/sciimmunol.aba5962.
Dobrzanski, Mark J. 2013. «Expanding Roles for CD4 T Cells and Their Subpopulations in Tumor Immunity and Therapy». Frontiers in Oncology 3: 63. https://doi.org/10.3389/fonc.2013.00063.
Doench, John G., Nicolo Fusi, Meagan Sullender, Mudra Hegde, Emma W. Vaimberg, Katherine F. Donovan, Ian Smith, et al. 2016. «Optimized SgRNA Design to Maximize Activity and Minimize Off-Target Effects of CRISPR-Cas9». Nature Biotechnology 34 (2): 184-91. https://doi.org/10.1038/nbt.3437.
Dong, Matthew B., Guangchuan Wang, Ryan D. Chow, Lupeng Ye, Lvyun Zhu, Xiaoyun Dai, Jonathan J. Park, et al. 2019. «Systematic Immunotherapy Target Discovery Using Genome-Scale In Vivo CRISPR Screens in CD8 T Cells». Cell 178 (5): 1189-1204.e23. https://doi.org/10.1016/j.cell.2019.07.044.
Egwuagu, Charles E., Cheng-Rong Yu, Meifen Zhang, Rashid M. Mahdi, Stephen J. Kim, et Igal Gery. 2002. «Suppressors of Cytokine Signaling Proteins Are Differentially Expressed in Th1 and Th2 Cells: Implications for Th Cell Lineage Commitment and Maintenance». Journal of Immunology (Baltimore, Md.: 1950) 168 (7): 3181-87. https://doi.org/10.4049/jimmunol.168.7.3181.
Elliott, T. J., et H. N. Eisen. 1988. «Allorecognition of Purified Major Histocompatibility Complex Glycoproteins by Cytotoxic T Lymphocytes». Proceedings of the National Academy of Sciences of the United States of America 85 (8): 2728-32. https://doi.org/10.1073/pnas.85.8.2728.
Eyquem, Justin, Jorge Mansilla-Soto, Theodoros Giavridis, Sjoukje J. C. van der Stegen, Mohamad Hamieh, Kristen M. Cunanan, Ashlesha Odak, Mithat Gonen, et Michel Sadelain. 2017. «Targeting a CAR to the TRAC Locus with CRISPR/Cas9 Enhances Tumour Rejection». Nature 543 (7643): 113-17. https://doi.org/10.1038/nature21405.
Forrester, J. V., H. Xu, T. Lambe, et R. Cornall. 2008. «Immune Privilege or Privileged Immunity?» Mucosal Immunology 1 (5): 372-81. https://doi.org/10.1038/mi.2008.27.
Foulds, Kathryn E., Lauren A. Zenewicz, Devon J. Shedlock, Jiu Jiang, Amy E. Troy, et Hao Shen. 2002. «Cutting Edge: CD4 and CD8 T Cells Are Intrinsically Different in Their Proliferative Responses». Journal of Immunology (Baltimore, Md.: 1950) 168 (4): 1528-32. https://doi.org/10.4049/jimmunol.168.4.1528.
Freitas, A. A., et B. Rocha. 2000. «Population Biology of Lymphocytes: The Flight for Survival». Annual Review of Immunology 18: 83-111. https://doi.org/10.1146/annurev.immunol.18.1.83.
Gattinoni, Luca, Steven E. Finkelstein, Christopher A. Klebanoff, Paul A. Antony, Douglas C. Palmer, Paul J. Spiess, Leroy N. Hwang, et al. 2005. «Removal of Homeostatic Cytokine Sinks by Lymphodepletion Enhances the Efficacy of Adoptively Transferred Tumor-Specific CD8+ T Cells». The Journal of Experimental Medicine 202 (7): 907-12. https://doi.org/10.1084/jem.20050732.
Gonen-Gross, Tsufit, Debra Goldman-Wohl, Berthold Huppertz, Dikla Lankry, Caryn Greenfield, Shira Natanson-Yaron, Yaron Hamani, Ronit Gilad, Simcha Yagel, et Ofer Mandelboim. 2010. «Inhibitory NK Receptor Recognition of HLA-G: Regulation by Contact Residues and by Cell Specific Expression at the Fetal-Maternal Interface». PloS One 5 (1): e8941. https://doi.org/10.1371/journal.pone.0008941.
Gornalusse, Germen G., Roli K. Hirata, Sarah E. Funk, Laura Riolobos, Vanda S. Lopes, Gabriel Manske, Donna Prunkard, et al. 2017. «HLA-E-Expressing Pluripotent Stem Cells Escape Allogeneic Responses and Lysis by NK Cells». Nature Biotechnology 35 (8): 765-72. https://doi.org/10.1038/nbt.3860.
Graham, Charlotte, Agnieszka Jozwik, Andrea Pepper, et Reuben Benjamin. 2018. «(Allogeneic CAR-T Cells: More than Ease of Access?» Cells 7 (10). https://doi.org/10.3390/cells7100155.
Hamieh, Mohamad, Anton Dobrin, Annalisa Cabriolu, Sjoukje J. C. van der Stegen, Theodoros Giavridis, Jorge Mansilla-Soto, Justin Eyquem, et al. 2019a. «CAR T Cell Trogocytosis and Cooperative Killing Regulate Tumour Antigen Escape». Nature 568 (7750): 112-16. https://doi.org/10.1038/s41586-019-1054-1.
______. 2019b. «CAR T Cell Trogocytosis and Cooperative Killing Regulate Tumour Antigen Escape». Nature 568 (7750): 112-16. https://doi.org/10.1038/s41586-019-1054-1.
He, Yayi, Leslie Rozeboom, Christopher J. Rivard, Kim Ellison, Rafal Dziadziuszko, Hui Yu, Caicun Zhou, et Fred R. Hirsch. 2017. «MHC Class II Expression in Lung Cancer». Lung Cancer (Amsterdam, Netherlands) 112: 75-80. https://doi.org/10.1016/j.lungcan.2017.07.030.
Helft, Julie, Alexandra Jacquet, Nathalie T. Joncker, Isabelle Grandjean, Guillaume Dorothée, Adrien Kissenpfennig, Bernard Malissen, Polly Matzinger, et Olivier Lantz. 2008. «Antigen-Specific T-T Interactions Regulate CD4 T-Cell Expansion». Blood 112 (4): 1249-58. https://doi.org/10.1182/blood-2007-09-114389.
Homann, D., L. Teyton, et M. B. Oldstone. 2001. «Differential Regulation of Antiviral T-Cell Immunity Results in Stable CD8+ but Declining CD4+ T-Cell Memory». Nature Medicine 7 (8): 913-19. https://doi.org/10.1038/90950.
Kagoya, Yuki, Shinya Tanaka, Tingxi Guo, Mark Anczurowski, Chung-Hsi Wang, Kayoko Saso, Marcus O. Butler, Mark D. Minden, et Naoto Hirano. 2018. «A Novel Chimeric Antigen Receptor Containing a JAK-STAT Signaling Domain Mediates Superior Antitumor Effects». Nature Medicine 24 (3): 352-59. https://doi.org/10.1038/nm.4478.
Kitano, Shigehisa, Takemasa Tsuji, Caillian Liu, Daniel Hirschhorn-Cymerman, Chrisann Kyi, Zhenyu Mu, James P. Allison, Sacha Gnjatic, Jianda D. Yuan, et Jedd D. Wolchok. 2013. «Enhancement of Tumor-Reactive Cytotoxic CD4⁺ T Cell Responses after Ipilimumab Treatment in Four Advanced Melanoma Patients». Cancer Immunology Research 1 (4): 235-44. https://doi.org/10.1158/2326-6066.CIR-13-0068.
Köhl, Ulrike, Stanislava Arsenieva, Astrid Holzinger, et Hinrich Abken. 2018. «CAR T Cells in Trials: Recent Achievements and Challenges That Remain in the Production of Modified T Cells for Clinical Applications». Human Gene Therapy 29 (5): 559-68. https://doi.org/10.1089/hum.2017.254.
Koopman, L. A., W. E. Corver, A. R. van der Slik, M. J. Giphart, et G. J. Fleuren. 2000. «Multiple Genetic Alterations Cause Frequent and Heterogeneous Human Histocompatibility Leukocyte Antigen Class I Loss in Cervical Cancer». The Journal of Experimental Medicine 191 (6): 961-76. https://doi.org/10.1084/jem.191.6.961.
Krueger, Peter D., Michael F. Goldberg, Sung-Wook Hong, Kevin C. Osum, Ryan A. Langlois, Dmitri I. Kotov, Thamotharampillai Dileepan, et Marc K. Jenkins. 2021. «Two Sequential Activation Modules Control the Differentiation of Protective T Helper-1 (Th1) Cells». Immunity 54 (4): 687-701.e4. https://doi.org/10.1016/j.immuni.2021.03.006.
Lanza, Robert, David W. Russell, et Andras Nagy. 2019. «Engineering Universal Cells That Evade Immune Detection». Nature Reviews. Immunology 19 (12): 723-33. https://doi.org/10.1038/s41577-019-0200-1.
Lenardo, M. J. 1991. «Interleukin-2 Programs Mouse Alpha Beta T Lymphocytes for Apoptosis». Nature 353 (6347): 858-61. https://doi.org/10.1038/353858a0.
Li, Hanjie, Anne M. van der Leun, Ido Yofe, Yaniv Lubling, Dikla Gelbard-Solodkin, Alexander C. J. van Akkooi, Marlous van den Braber, et al. 2019. «Dysfunctional CD8 T Cells Form a Proliferative, Dynamically Regulated Compartment within Human Melanoma». Cell 176 (4): 775-789.e18. https://doi.org/10.1016/j.cell.2018.11.043.
Li, Wei, Johannes Köster, Han Xu, Chen-Hao Chen, Tengfei Xiao, Jun S. Liu, Myles Brown, et X. Shirley Liu. 2015. «Quality Control, Modeling, and Visualization of CRISPR Screens with MAGeCK-VISPR». Genome Biology 16 (1). https://doi.org/10.1186/s13059-015-0843-6.
Lim, Wendell A., et Carl H. June. 2017. «The Principles of Engineering Immune Cells to Treat Cancer». Cell 168 (4): 724-40. https://doi.org/10.1016/j.cell.2017.01.016.
Lin, John K., Lori S. Muffly, Michael A. Spinner, James I. Barnes, Douglas K. Owens, et Jeremy D. Goldhaber-Fiebert. 2019. «Cost Effectiveness of Chimeric Antigen Receptor T-Cell Therapy in Multiply Relapsed or Refractory Adult Large B-Cell Lymphoma». Journal of Clinical Oncology: Official Journal of the American Society of Clinical Oncology 37 (24): 2105-19. https://doi.org/10.1200/JCO.18.02079.
Linnemann, Carsten, Ton N. M. Schumacher, et Gavin M. Bendle. 2011. «T-Cell Receptor Gene Therapy: Critical Parameters for Clinical Success». The Journal of Investigative Dermatology 131 (9): 1806-16. https://doi.org/10.1038/jid.2011.160.
MacLeod, Megan K. L., John W. Kappler, et Philippa Marrack. 2010. «Memory CD4 T Cells: Generation, Reactivation and Re-Assignment». Immunology 130 (1): 10-15. https://doi.org/10.1111/j.1365-2567.2010.03260.x.
Majri, Sonia S., Jill M. Fritz, Alejandro V. Villarino, Lixin Zheng, Chrysi Kanellopoulou, Benjamin Chaigne-Delalande, Juha Gronholm, et al. 2018. «STAT5B: A Differential Regulator of the Life and Death of CD4+ Effector Memory T Cells». Journal of Immunology (Baltimore, Md.: 1950) 200 (1): 110-18. https://doi.org/10.4049/jimmunol.1701133.
Markley, John C., et Michel Sadelain. 2010. «IL-7 and IL-21 Are Superior to IL-2 and IL-15 in Promoting Human T Cell-Mediated Rejection of Systemic Lymphoma in Immunodeficient Mice». Blood 115 (17): 3508-19. https://doi.org/10.1182/blood-2009-09-241398.
Maude, Shannon L., Theodore W. Laetsch, Jochen Buechner, Susana Rives, Michael Boyer, Henrique Bittencourt, Peter Bader, et al. 2018. «Tisagenlecleucel in Children and Young Adults with B-Cell Lymphoblastic Leukemia». The New England Journal of Medicine 378 (5): 439-48. https://doi.org/10.1056/NEJMoa1709866.
Merica, R., A. Khoruts, K. A. Pape, R. L. Reinhardt, et M. K. Jenkins. 2000. «Antigen-Experienced CD4 T Cells Display a Reduced Capacity for Clonal Expansion in Vivo That Is Imposed by Factors Present in the Immune Host». Journal of Immunology (Baltimore, Md.: 1950) 164 (9): 4551-57. https://doi.org/10.4049/jimmunol.164.9.4551.
Mo, Feiyan, Norihiro Watanabe, Mary K. McKenna, M. John Hicks, Madhuwanti Srinivasan, Diogo Gomes-Silva, Erden Atilla, et al. 2020. «Engineered Off-the-Shelf Therapeutic T Cells Resist Host Immune Rejection». Nature Biotechnology, juillet. https://doi.org/10.1038/s41587-020-0601-5.
Monteverde, T., J. Tait-Mulder, A. Hedley, J. R. Knight, O. J. Sansom, et D. J. Murphy. 2018. «Calcium Signalling Links MYC to NUAK1». Oncogene 37 (8): 982-92. https://doi.org/10.1038/onc.2017.394.
Neelapu, Sattva S., Frederick L. Locke, Nancy L. Bartlett, Lazaros J. Lekakis, David B. Miklos, Caron A. Jacobson, Ira Braunschweig, et al. 2017. «Axicabtagene Ciloleucel CAR T-Cell Therapy in Refractory Large B-Cell Lymphoma». The New England Journal of Medicine 377 (26): 2531-44. https://doi.org/10.1056/NEJMoa1707447.
Nianias, Alexandros, et Maria Themeli. 2019. «Induced Pluripotent Stem Cell (IPSC)-Derived Lymphocytes for Adoptive Cell Immunotherapy: Recent Advances and Challenges». Current Hematologic Malignancy Reports 14 (4): 261-68. https://doi.org/10.1007/s11899-019-00528-6.
Palmer, Douglas C., Beau R. Webber, Yogin Patel, Matthew J. Johnson, Christine M. Kariya, Walker S. Lahr, Maria R. Parkhurst, et al. 2020. «Internal checkpoint regulates T cell neoantigen reactivity and susceptibility to PD1 blockade». bioRxiv, janvier, 2020.09.24.306571. https://doi.org/10.1101/2020.09.24.306571.
Pazmany, L., O. Mandelboim, M. Valés-Gómez, D. M. Davis, H. T. Reyburn, et J. L. Strominger. 1996. «Protection from Natural Killer Cell-Mediated Lysis by HLA-G Expression on Target Cells». Science (New York, N. Y.) 274 (5288): 792-95. https://doi.org/10.1126/science.274.5288.792.
Poirot, Laurent, Brian Philip, Cécile Schiffer-Mannioui, Diane Le Clerre, Isabelle Chion-Sotinel, Sophie Derniame, Pierrick Potrel, et al. 2015. «Multiplex Genome-Edited T-Cell Manufacturing Platform for “Off-the-Shelf” Adoptive T-Cell Immunotherapies». Cancer Research 75 (18): 3853-64. https://doi.org/10.1158/0008-5472.CAN-14-3321.
Quezada, Sergio A., Tyler R. Simpson, Karl S. Peggs, Taha Merghoub, Jelena Vider, Xiaozhou Fan, Ronald Blasberg, et al. 2010. «Tumor-Reactive CD4(+) T Cells Develop Cytotoxic Activity and Eradicate Large Established Melanoma after Transfer into Lymphopenic Hosts». The Journal of Experimental Medicine 207 (3): 637-50. https://doi.org/10.1084/jem.20091918.
Quintarelli, Concetta, Juan F. Vera, Barbara Savoldo, Greta M. P. Giordano Attianese, Martin Pule, Aaron E. Foster, Helen E. Heslop, Cliona M. Rooney, Malcolm K. Brenner, et Gianpietro Dotti. 2007. «Co-Expression of Cytokine and Suicide Genes to Enhance the Activity and Safety of Tumor-Specific Cytotoxic T Lymphocytes». Blood 110 (8): 2793-2802. https://doi.org/10.1182/blood-2007-02-072843.
Rådestad, E., H. Wikell, M. Engström, E. Watz, B. Sundberg, S. Thunberg, M. Uzunel, J. Mattsson, et M. Uhlin. 2014. «Alpha/Beta T-Cell Depleted Grafts as an Immunological Booster to Treat Graft Failure after Hematopoietic Stem Cell Transplantation with HLA-Matched Related and Unrelated Donors». Journal of Immunology Research 2014: 578741. https://doi.org/10.1155/2014/578741.
Rajagopalan, S., et E. O. Long. 1999. «A Human Histocompatibility Leukocyte Antigen (HLA)-G-Specific Receptor Expressed on All Natural Killer Cells». The Journal of Experimental Medicine 189 (7): 1093-1100. https://doi.org/10.1084/jem.189.7.1093.
Ramanathan, Sheela, Stephanie Dubois, Julien Gagnon, Chantal Leblanc, Sanjeev Mariathasan, Gerardo Ferbeyre, Robert Rottapel, Pamela S. Ohashi, et Subburaj Ilangumaran. 2010. «Regulation of Cytokine-Driven Functional Differentiation of CD8 T Cells by Suppressor of Cytokine Signaling 1 Controls Autoimmunity and Preserves Their Proliferative Capacity toward Foreign Antigens». Journal of Immunology (Baltimore, Md.: 1950) 185 (1): 357-66. https://doi.org/10.4049/jimmunol.1000066.
Ravkov, Eugene V., et Matthew A. Williams. 2009. «The Magnitude of CD4+ T Cell Recall Responses Is Controlled by the Duration of the Secondary Stimulus». Journal of Immunology (Baltimore, Md.: 1950) 183 (4): 2382-89. https://doi.org/10.4049/jimmunol.0900319.
Ren, Bing, Hieu Cam, Yasuhiko Takahashi, Thomas Volkert, Jolyon Terragni, Richard A. Young, et Brian David Dynlacht. 2002. «E2F Integrates Cell Cycle Progression with DNA Repair, Replication, and G(2)/M Checkpoints». Genes & Development 16 (2): 245-56. https://doi.org/10.1101/gad.949802.
Ruggeri, Loredana, Marusca Capanni, Elena Urbani, Katia Perruccio, Warren D. Shlomchik, Antonella Tosti, Sabrina Posati, et al. 2002. «Effectiveness of Donor Natural Killer Cell Alloreactivity in Mismatched Hematopoietic Transplants». Science (New York, N. Y.) 295 (5562): 2097-2100. https://doi.org/10.1126/science.1068440.
Sadelain, Michel. 2015. «CAR Therapy: The CD19 Paradigm». The Journal of Clinical Investigation 125 (9): 3392-3400. https://doi.org/10.1172/JC180010.
Sckisel, Gail D., Myriam N. Bouchlaka, Arta M. Monjazeb, Marka Crittenden, Brendan D. Curti, Danice E. C. Wilkins, Kory A. Alderson, et al. 2015. «Out-of-Sequence Signal 3 Paralyzes Primary CD4(+) T-Cell-Dependent Immunity». Immunity 43 (2): 240-50. https://doi.org/10.1016/j.immuni.2015.06.023.
Seder, Robert A., et Rafi Ahmed. 2003. «Similarities and Differences in CD4+ and CD8+ Effector and Memory T Cell Generation». Nature Immunology 4 (9): 835-42. https://doi.org/10.1038/ni969.
Shifrut, Eric, Julia Carnevale, Victoria Tobin, Theodore L. Roth, Jonathan M. Woo, Christina T. Bui, P. Jonathan Li, Morgan E. Diolaiti, Alan Ashworth, et Alexander Marson. 2018. «Genome-Wide CRISPR Screens in Primary Human T Cells Reveal Key Regulators of Immune Function». Cell 175 (7): 1958-1971.e15. https://doi.org/10.1016/j.cell.2018.10.024.
Śledzińska, Anna, Maria Vila de Mucha, Katharina Bergerhoff, Alastair Hotblack, Dafne Franz Demane, Ehsan Ghorani, Ayse U. Akarca, et al. 2020a. «Regulatory T Cells Restrain Interleukin-2- and Blimp-1-Dependent Acquisition of Cytotoxic Function by CD4+ T Cells». Immunity 52 (1): 151-166.e6. https://doi.org/10.1016/j.immuni.2019.12.007.
______. 2020b. «Regulatory T Cells Restrain Interleukin-2- and Blimp-1-Dependent Acquisition of Cytotoxic Function by CD4+ T Cells». Immunity 52 (1): 151-166.e6. https://doi.org/10.1016/j.immuni.2019.12.007.
Takahashi, Reiko, Hiroko Nakatsukasa, Shunichi Shiozawa, et Akihiko Yoshimura. 2017. «SOCS1 Is a Key Molecule That Prevents Regulatory T Cell Plasticity under Inflammatory Conditions». Journal of Immunology (Baltimore, Md.: 1950) 199 (1): 149-58. https://doi.org/10.4049/jimmunol.1600441.
Takahashi, Reiko, Shuhei Nishimoto, Go Muto, Takashi Sekiya, Taiga Tamiya, Akihiro Kimura, Rimpei Morita, Mayako Asakawa, Takatoshi Chinen, et Akihiko Yoshimura. 2011. «SOCS1 Is Essential for Regulatory T Cell Functions by Preventing Loss of Foxp3 Expression as Well as IFN-{gamma} and IL-17A Production». The Journal of Experimental Medicine 208 (10): 2055-67. https://doi.org/10.1084/jem.20110428.
Teijaro, John R., Cherie Ng, Andrew M. Lee, Brian M. Sullivan, Kathleen C. F. Sheehan, Megan Welch, Robert D. Schreiber, Juan Carlos de la Torre, et Michael B. A. Oldstone. 2013. «Persistent LCMV Infection Is Controlled by Blockade of Type I Interferon Signaling». Science (New York, N. Y.) 340 (6129): 207-11. https://doi.org/10.1126/science.1235214.
Themeli, Maria, Christopher C. Kloss, Giovanni Ciriello, Victor D. Fedorov, Fabiana Perna, Mithat Gonen, et Michel Sadelain. 2013. «Generation of Tumor-Targeted Human T Lymphocytes from Induced Pluripotent Stem Cells for Cancer Therapy». Nature Biotechnology 31 (10): 928-33. https://doi.org/10.1038/nbt.2678.
Thommen, Daniela S., et Ton N. Schumacher. 2018. «T Cell Dysfunction in Cancer». Cancer Cell 33 (4): 547-62. https://doi.org/10.1016/j.ccell.2018.03.012.
Torikai, Hiroki, Andreas Reik, Frank Soldner, Edus H. Warren, Carrie Yuen, Yuanyue Zhou, Denise L. Crossland, et al. 2013. «Toward Eliminating HLA Class I Expression to Generate Universal Cells from Allogeneic Donors». Blood 122 (8): 1341-49. https://doi.org/10.1182/blood-2013-03-478255.
Vacchio, Melanie S., et Richard J. Hodes. 2005. «Fetal Expression of Fas Ligand Is Necessary and Sufficient for Induction of CD8 T Cell Tolerance to the Fetal Antigen H-Y during Pregnancy». Journal of Immunology (Baltimore, Md.: 1950) 174 (8): 4657-61. https://doi.org/10.4049/jimmunol.174.8.4657.
Valton, Julien, Valérie Guyot, Alan Marechal, Jean-Marie Filhol, Alexandre Juillerat, Aymeric Duclert, Philippe Duchateau, et Laurent Poirot. 2015. «A Multidrug-Resistant Engineered CAR T Cell for Allogeneic Combination Immunotherapy». Molecular Therapy: The Journal of the American Society of Gene Therapy 23 (9): 1507-18. https://doi.org/10.1038/mt.2015.104.
Wang, Dachun, Yuan Quan, Qing Yan, John E. Morales, et Rick A. Wetsel. 2015. «Targeted Disruption of the B2-Microglobulin Gene Minimizes the Immunogenicity of Human Embryonic Stem Cells». Stem Cells Translational Medicine 4 (10): 1234-45. https://doi.org/10.5966/sctm.2015-0049.
Wang, Tao, Catherine Huang, Alfonso Lopez-Coral, Kimberly A. Slentz-Kesler, Min Xiao, E. John Wherry, et Russel E. Kaufman. 2012. «K12/SECTM1, an Interferon-γ Regulated Molecule, Synergizes with CD28 to Costimulate Human T Cell Proliferation». Journal of Leukocyte Biology 91 (3): 449-59. https://doi.org/10.1189/jlb.1011498.
Wei, Jun, Lingyun Long, Wenting Zheng, Yogesh Dhungana, Seon Ah Lim, Cliff Guy, Yanyan Wang, et al. 2019. «Targeting REGNASE-1 Programs Long-Lived Effector T Cells for Cancer Therapy». Nature 576 (7787): 471-76. https://doi.org/10.1038/s41586-019-1821-z.
Wherry, E. John, et Makoto Kurachi. 2015. «Molecular and Cellular Insights into T Cell Exhaustion». Nature Reviews. Immunology 15 (8): 486-99. https://doi.org/10.1038/nri3862.
Wilson, Elizabeth B., Douglas H. Yamada, Heidi Elsaesser, Jonathan Herskovitz, Jane Deng, Genhong Cheng, Bruce J. Aronow, Christopher L. Karp, et David G. Brooks. 2013. «Blockade of Chronic Type I Interferon Signaling to Control Persistent LCMV Infection». Science (New York, N. Y.) 340 (6129): 202-7. https://doi.org/10.1126/science.1235208.
Xie, Ying, Akguil Akpinarli, Charles Maris, Edward L. Hipkiss, Malcolm Lane, Eun-Kyung M. Kwon, Pawel Muranski, Nicholas P. Restifo, et Paul Andrew Antony. 2010. «Naive Tumor-Specific CD4(+) T Cells Differentiated in Vivo Eradicate Established Melanoma». The Journal of Experimental Medicine 207 (3): 651-67. https://doi.org/10.1084/jem.20091921.
Zhou, Penghui, Donald R. Shaffer, Diana A. Alvarez Arias, Yukoh Nakazaki, Wouter Pos, Alexis J. Torres, Viviana Cremasco, et al. 2014. «In Vivo Discovery of Immunotherapy Targets in the Tumour Microenvironment». Nature 506 (7486): 52-57. https://doi.org/10.1038/nature12988.
Zhu, J. W., S. J. Field, L. Gore, M. Thompson, H. Yang, Y. Fujiwara, R. D. Cardiff, M. Greenberg, S. H. Orkin, et J. DeGregori. 2001. «E2F1 and E2F2 Determine Thresholds for Antigen-Induced T-Cell Proliferation and Suppress Tumorigenesis». Molecular and Cellular Biology 21 (24): 8547-64. https://doi.org/10.1128/MCB.21.24.8547-8564.2001.
Zhu, Ziqiang, Steven M. Cuss, Vinod Singh, Devikala Gurusamy, Jennifer L. Shoe, Robert Leighty, Vincenzo Bronte, et Arthur A. Hurwitz. 2015. «CD4+ T Cell Help Selectively Enhances High-Avidity Tumor Antigen-Specific CD8+ T Cells». Journal of Immunology (Baltimore, Md.: 1950) 195 (7): 3482-89. https://doi.org/10.4049/jimmunol.1401571.
Albrecht, Inka, Uwe Niesner, Marko Janke, Astrid Menning, Christoph Loddenkemper, Anja A. Kühl, Inga Lepenies, et al. 2010. «Persistence of Effector Memory Th1 Cells Is Regulated by Hopx». European Journal of Immunology 40 (11): 2993-3006. https://doi.org/10.1002/eji.201040936.
Alonso, Ruby, Héloise Flament, Sébastien Lemoine, Christine Sedlik, Emanuel Bottasso, Isabel Péguillet, Virginie Prémel, et al. 2018. «Induction of Anergic or Regulatory Tumor-Specific CD4+ T Cells in the Tumor-Draining Lymph Node». Nature Communications 9 (1): 2113. https://doi.org/10.1038/s41467-018-04524-x.
Bern, Michael D., Bijal A. Parikh, Liping Yang, Diana L. Beckman, Jennifer Poursine-Laurent, et Wayne M. Yokoyama. 2019. «Inducible Down-Regulation of MHC Class I Results in Natural Killer Cell Tolerance». The Journal of Experimental Medicine 216 (1): 99-116. https://doi.org/10.1084/jem.20181076.
Berner, Vanessa, Haiyan Liu, Qing Zhou, Kory L. Alderson, Kai Sun, Jonathan M. Weiss, Timothy C. Back, et al. 2007. «IFN-Gamma Mediates CD4+ T-Cell Loss and Impairs Secondary Antitumor Responses after Successful Initial Immunotherapy». Nature Medicine 13 (3): 354-60. https://doi.org/10.1038/nm1554.
Bix, M., N. S. Liao, M. Zijlstra, J. Loring, R. Jaenisch, et D. Raulet. 1991. «Rejection of Class I MHC-Deficient Haemopoietic Cells by Irradiated MHC-Matched Mice». Nature 349 (6307): 329-31. https://doi.org/10.1038/349329a0.
Boni, Andrea, Pawel Muranski, Lydie Cassard, Claudia Wrzesinski, Chrystal M. Paulos, Douglas C. Palmer, Luca Gattinoni, et al. 2008. «Adoptive Transfer of Allogeneic Tumor-Specific T Cells Mediates Effective Regression of Large Tumors across Major Histocompatibility Barriers». Blood 112 (12): 4746-54. https://doi.org/10.1182/blood-2008-07-169797.
Borst, Jannie, Tomasz Ahrends, Nikolina Bqbata, Cornelis J. M. Melief, et Wolfgang Kastenmuller. 2018. «CD4+ T Cell Help in Cancer Immunology and Immunotherapy». Nature Reviews. Immunology 18 (10): 635-47. https://doi.org/10.1038/s41577-018-0044-0.
Bos, Rinke, et Linda A. Sherman. 2010. «CD4+ T-Cell Help in the Tumor Milieu Is Required for Recruitment and Cytolytic Function of CD8+T Lymphocytes». Cancer Research 70 (21): 8368-77. https://doi.org/10.1158/0008-5472.CAN-10-1322.
Braud, V. M., D. S. Allan, C. A. O'Callaghan, K. Söderström, A. D'Andrea, G. S. Ogg, S. Lazetic, et al. 1998. «HLA-E Binds to Natural Killer Cell Receptors CD94/NKG2A, B and C». Nature 391 (6669): 795-99. https://doi.org/10.1038/35869.
Chakrabarti, S., G. Hale, et H. Waldmann. 2004. «Alemtuzumab (Campath-1H) in Allogeneic Stem Cell Transplantation: Where Do We Go from Here?» Transplantation Proceedings 36 (5): 1225-27. https://doi.org/10.1016/j.transproceed.2004.05.067.
Champlin, R. E., J. R. Passweg, M. J. Zhang, P. A. Rowlings, C. J. Pelz, K. A. Atkinson, A. J. Barrett, et al. 2000. «T-Cell Depletion of Bone Marrow Transplants for Leukemia from Donors Other than HLA-Identical Siblings: Advantage of T-Cell Antibodies with Narrow Specificities». Blood 95 (12): 3996-4003.
Ciccone, E., D. Pende, O. Viale, C. Di Donato, G. Tripodi, A. M. Orengo, J. Guardiola, A. Moretta, et L. Moretta. 1992. «Evidence of a Natural Killer (NK) Cell Repertoire for (Allo) Antigen Recognition: Definition of Five Distinct NK-Determined Allospecificities in Humans». The Journal of Experimental Medicine 175 (3): 709-18. https://doi.org/10.1084/jem.175.3.709.
Corthay, Alexandre, Dag K. Skovseth, Katrin U. Lundin, Egil Rosjo, Hilde Omholt, Peter O. Hofgaard, Guttorm Haraldsen, et Bjarne Bogen. 2005. «Primary Antitumor Immune Response Mediated by CD4+ T Cells». Immunity 22 (3): 371-83. https://doi.org/10.1016/j.immuni.2005.02.003.
Del Galy, Aurélien Sutra, Silvia Menegatti, Jaime Fuentealba, Laetitia Perrin, Francesca Lucibello, Julie Helft, Aurélie Darbois, et al. 2021. «In vivo genome-wide CRISPR screens identify SOCS1 as a major intrinsic checkpoint of CD4⁺ Th1 cell response». bioRxiv, janvier, 2021.04.12.439455. https://doi.org/10.1101/2021.04.12.439455.
Depil, S., P. Duchateau, S. A. Grupp, G. Mufti, et L. Poirot. 2020. «“Off-the-Shelf” Allogeneic CAR T Cells: Development and Challenges». Nature Reviews. Drug Discovery, janvier. https://doi.org/10.1038/s41573-019-0051-2.
Ding, Huihua, et Tianfu Wu. 2018. «Insulin-Like Growth Factor Binding Proteins in Autoimmune Diseases». Frontiers in Endocrinology 9: 499. https://doi.org/10.3389/fendo.2018.00499.
Ding, Zhi-Chun, Huidong Shi, Nada S. Aboelella, Kateryna Fesenkova, Eun-Jeong Park, Zhuoqi Liu, Lirong Pei, et al. 2020. «Persistent STAT5 Activation Reprograms the Epigenetic Landscape in CD4+ T Cells to Drive Polyfunctionality and Antitumor Immunity». Science Immunology 5 (52). https://doi.org/10.1126/sciimmunol.aba5962.
Dobrzanski, Mark J. 2013. «Expanding Roles for CD4 T Cells and Their Subpopulations in Tumor Immunity and Therapy». Frontiers in Oncology 3: 63. https://doi.org/10.3389/fonc.2013.00063.
Doench, John G., Nicolo Fusi, Meagan Sullender, Mudra Hegde, Emma W. Vaimberg, Katherine F. Donovan, Ian Smith, et al. 2016. «Optimized SgRNA Design to Maximize Activity and Minimize Off-Target Effects of CRISPR-Cas9». Nature Biotechnology 34 (2): 184-91. https://doi.org/10.1038/nbt.3437.
Dong, Matthew B., Guangchuan Wang, Ryan D. Chow, Lupeng Ye, Lvyun Zhu, Xiaoyun Dai, Jonathan J. Park, et al. 2019. «Systematic Immunotherapy Target Discovery Using Genome-Scale In Vivo CRISPR Screens in CD8 T Cells». Cell 178 (5): 1189-1204.e23. https://doi.org/10.1016/j.cell.2019.07.044.
Egwuagu, Charles E., Cheng-Rong Yu, Meifen Zhang, Rashid M. Mahdi, Stephen J. Kim, et Igal Gery. 2002. «Suppressors of Cytokine Signaling Proteins Are Differentially Expressed in Th1 and Th2 Cells: Implications for Th Cell Lineage Commitment and Maintenance». Journal of Immunology (Baltimore, Md.: 1950) 168 (7): 3181-87. https://doi.org/10.4049/jimmunol.168.7.3181.
Elliott, T. J., et H. N. Eisen. 1988. «Allorecognition of Purified Major Histocompatibility Complex Glycoproteins by Cytotoxic T Lymphocytes». Proceedings of the National Academy of Sciences of the United States of America 85 (8): 2728-32. https://doi.org/10.1073/pnas.85.8.2728.
Eyquem, Justin, Jorge Mansilla-Soto, Theodoros Giavridis, Sjoukje J. C. van der Stegen, Mohamad Hamieh, Kristen M. Cunanan, Ashlesha Odak, Mithat Gönen, et Michel Sadelain. 2017. «Targeting a CAR to the TRAC Locus with CRISPR/Cas9 Enhances Tumour Rejection». Nature 543 (7643): 113-17. https://doi.org/10.1038/nature21405.
Forrester, J. V., H. Xu, T. Lambe, et R. Cornall. 2008. «Immune Privilege or Privileged Immunity?» Mucosal Immunology 1 (5): 372-81. https://doi.org/10.1038/mi.2008.27.
Foulds, Kathryn E., Lauren A. Zenewicz, Devon J. Shedlock, Jiu Jiang, Amy E. Troy, et Hao Shen. 2002. «Cutting Edge: CD4 and CD8 T Cells Are Intrinsically Different in Their Proliferative Responses». Journal of Immunology (Baltimore, Md.: 1950) 168 (4): 1528-32. https://doi.org/10.4049/jimmunol.168.4.1528.
Freitas, A. A., et B. Rocha. 2000. «Population Biology of Lymphocytes: The Flight for Survival». Annual Review of Immunology 18: 83-111. https://doi.org/10.1146/annurev.immunol.18.1.83.
Gattinoni, Luca, Steven E. Finkelstein, Christopher A. Klebanoff, Paul A. Antony, Douglas C. Palmer, Paul J. Spiess, Leroy N. Hwang, et al. 2005. «Removal of Homeostatic Cytokine Sinks by Lymphodepletion Enhances the Efficacy of Adoptively Transferred Tumor-Specific CD8+ T Cells». The Journal of Experimental Medicine 202 (7): 907-12. https://doi.org/10.1084/jem.20050732.
Gonen-Gross, Tsufit, Debra Goldman-Wohl, Berthold Huppertz, Dikla Lankry, Caryn Greenfield, Shira Natanson-Yaron, Yaron Hamani, Ronit Gilad, Simcha Yagel, et Ofer Mandelboim. 2010. «Inhibitory NK Receptor Recognition of HLA-G: Regulation by Contact Residues and by Cell Specific Expression at the Fetal-Maternal Interface». PloS One 5 (1): e8941. https://doi.org/10.1371/journal.pone.0008941.
Gornalusse, Germen G., Roli K. Hirata, Sarah E. Funk, Laura Riolobos, Vanda S. Lopes, Gabriel Manske, Donna Prunkard, et al. 2017. «HLA-E-Expressing Pluripotent Stem Cells Escape Allogeneic Responses and Lysis by NK Cells». Nature Biotechnology 35 (8): 765-72. https://doi.org/10.1038/nbt.3860.
Graham, Charlotte, Agnieszka Jozwik, Andrea Pepper, et Reuben Benjamin. 2018. «Allogeneic CAR-T Cells: More than Ease of Access?». Cells 7 (10). https://doi.org/10.3390/cells7100155.
Hamieh, Mohamad, Anton Dobrin, Annalisa Cabriolu, Sjoukje J. C. van der Stegen, Theodoros Giavridis, Jorge Mansilla-Soto, Justin Eyquem, et al. 2019a. «CAR T Cell Trogocytosis and Cooperative Killing Regulate Tumour Antigen Escape». Nature 568 (7750): 112-16. https://doi.org/10.1038/s41586-019-1054-1.
______. 2019b. «CAR T Cell Trogocytosis and Cooperative Killing Regulate Tumour Antigen Escape». Nature 568 (7750): 112-16. https://doi.org/10.1038/s41586-019-1054-1.
He, Yayi, Leslie Rozeboom, Christopher J. Rivard, Kim Ellison, Rafal Dziadziuszko, Hui Yu, Caicun Zhou, et Fred R. Hirsch. 2017. «MHC Class II Expression in Lung Cancer». Lung Cancer (Amsterdam, Netherlands) 112: 75-80. https://doi.org/10.1016/j.lungcan.2017.07.030.
Helft, Julie, Alexandra Jacquet, Nathalie T. Joncker, Isabelle Grandjean, Guillaume Dorothée, Adrien Kissenpfennig, Bernard Malissen, Polly Matzinger, et Olivier Lantz. 2008. «Antigen-Specific T-T Interactions Regulate CD4 T-Cell Expansion». Blood 112 (4): 1249-58. https://doi.org/10.1182/blood-2007-09-114389.
Homann, D., L. Teyton, et M. B. Oldstone. 2001. «Differential Regulation of Antiviral T-Cell Immunity Results in Stable CD8+ but Declining CD4+ T-Cell Memory». Nature Medicine 7 (8): 913-19. https://doi.org/10.1038/90950.
Kagoya, Yuki, Shinya Tanaka, Tingxi Guo, Mark Anczurowski, Chung-Hsi Wang, Kayoko Saso, Marcus O. Butler, Mark D. Minden, et Naoto Hirano. 2018. «A Novel Chimeric Antigen Receptor Containing a JAK-STAT Signaling Domain Mediates Superior Antitumor Effects». Nature Medicine 24 (3): 352-59. https://doi.org/10.1038/nm.4478.
Kitano, Shigehisa, Takemasa Tsuji, Caillian Liu, Daniel Hirschhorn-Cymerman, Chrisann Kyi, Zhenyu Mu, James P. Allison, Sacha Gnjatic, Jianda D. Yuan, et Jedd D. Wolchok. 2013. «Enhancement of Tumor-Reactive Cytotoxic CD4+ T Cell Responses after Ipilimumab Treatment in Four Advanced Melanoma Patients». Cancer Immunology Research 1 (4): 235-44. https://doi.org/10.1158/2326-6066.CIR-13-0068.
Köhl, Ulrike, Stanislava Arsenieva, Astrid Holzinger, et Hinrich Abken. 2018. «CAR T Cells in Trials: Recent Achievements and Challenges That Remain in the Production of Modified T Cells for Clinical Applications». Human Gene Therapy 29 (5): 559-68. https://doi.org/10.1089/hum.2017.254.
Koopman, L. A., W. E. Corver, A. R. van der Slik, M. J. Giphart, et G. J. Fleuren. 2000. «Multiple Genetic Alterations Cause Frequent and Heterogeneous Human Histocompatibility Leukocyte Antigen Class I Loss in Cervical Cancer». The Journal of Experimental Medicine 191 (6): 961-76. https://doi.org/10.1084/jem.191.6.961.
Krueger, Peter D., Michael F. Goldberg, Sung-Wook Hong, Kevin C. Osum, Ryan A. Langlois, Dmitri I. Kotov, Thamotharampillai Dileepan, et Marc K. Jenkins. 2021. «Two Sequential Activation Modules Control the Differentiation of Protective T Helper-1 (Th1) Cells». Immunity 54 (4): 687-701.e4. https://doi.org/10.1016/j.immuni.2021.03.006.
Lanza, Robert, David W. Russell, et Andras Nagy. 2019. «Engineering Universal Cells That Evade Immune Detection». Nature Reviews. Immunology 19 (12): 723-33. https://doi.org/10.1038/s41577-019-0200-1.
Lenardo, M. J. 1991. «Interleukin-2 Programs Mouse Alpha Beta T Lymphocytes for Apoptosis». Nature 353 (6347): 858-61. https://doi.org/10.1038/353858a0.
Li, Hanjie, Anne M. van der Leun, Ido Yofe, Yaniv Lubling, Dikla Gelbard-Solodkin, Alexander C. J. van Akkooi, Marlous van den Braber, et al. 2019. «Dysfunctional CD8 T Cells Form a Proliferative, Dynamically Regulated Compartment within Human Melanoma». Cell 176 (4): 775-789.e18. https://doi.org/10.1016/j.cell.2018.11.043.
Li, Wei, Johannes Kóster, Han Xu, Chen-Hao Chen, Tengfei Xiao, Jun S. Liu, Myles Brown, et X. Shirley Liu. 2015. «Quality Control, Modeling, and Visualization of CRISPR Screens with MAGeCK-VISPR». Genome Biology 16 (1). https://doi.org/10.1186/s13059-015-0843-6.
Lim, Wendell A., et Carl H. June. 2017. «The Principles of Engineering Immune Cells to Treat Cancer». Cell 168 (4): 724-40. https://doi.org/10.1016/j.cell.2017.01.016.
Lin, John K., Lori S. Muffly, Michael A. Spinner, James I. Barnes, Douglas K. Owens, et Jeremy D. Goldhaber-Fiebert. 2019. «Cost Effectiveness of Chimeric Antigen Receptor T-Cell Therapy in Multiply Relapsed or Refractory Adult Large B-Cell Lymphoma». Journal of Clinical Oncology: Official Journal of the American Society of Clinical Oncology 37 (24): 2105-19. https://doi.org/10.1200/JCO.18.02079.
Linnemann, Carsten, Ton N. M. Schumacher, et Gavin M. Bendle. 2011. «T-Cell Receptor Gene Therapy: Critical Parameters for Clinical Success». The Journal of Investigative Dermatology 131 (9): 1806-16. https://doi.org/10.1038/jid.2011.160.
MacLeod, Megan K. L., John W. Kappler, et Philippa Marrack. 2010. «Memory CD4 T Cells: Generation, Reactivation and Re-Assignment». Immunology 130 (1): 10-15. https://doi.org/10.1111/j.1365-2567.2010.03260.x.
Majri, Sonia S., Jill M. Fritz, Alejandro V. Villarino, Lixin Zheng, Chrysi Kanellopoulou, Benjamin Chaigne-Delalande, Juha Gronholm, et al. 2018. «STAT5B: A Differential Regulator of the Life and Death of CD4+ Effector Memory T Cells». Journal of Immunology (Baltimore, Md.: 1950) 200 (1): 110-18. https://doi.org/10.4049/jimmunol.1701133.
Markley, John C., et Michel Sadelain. 2010. «IL-7 and IL-21 Are Superior to IL-2 and IL-15 in Promoting Human T Cell-Mediated Rejection of Systemic Lymphoma in Immunodeficient Mice». Blood 115 (17): 3508-19. https://doi.org/10.1182/blood-2009-09-241398.
Maude, Shannon L., Theodore W. Laetsch, Jochen Buechner, Susana Rives, Michael Boyer, Henrique Bittencourt, Peter Bader, et al. 2018. «Tisagenlecleucel in Children and Young Adults with B-Cell Lymphoblastic Leukemia». The New England Journal of Medicine 378 (5): 439-48. https://doi.org/10.1056/NEJMoa1709866.
Merica, R., A. Khoruts, K. A. Pape, R. L. Reinhardt, et M. K. Jenkins. 2000. «Antigen-Experienced CD4 T Cells Display a Reduced Capacity for Clonal Expansion in Vivo That Is Imposed by Factors Present in the Immune Host». Journal of Immunology (Baltimore, Md.: 1950) 164 (9): 4551-57. https://doi.org/10.4049/jimmunol.164.9.4551.
Mo, Feiyan, Norihiro Watanabe, Mary K. McKenna, M. John Hicks, Madhuwanti Srinivasan, Diogo Gomes-Silva, Erden Atilla, et al. 2020. «Engineered Off-the-Shelf Therapeutic T Cells Resist Host Immune Rejection». Nature Biotechnology, juillet. https://doi.org/10.1038/s41587-020-0601-5.
Monteverde, T., J. Tait-Mulder, A. Hedley, J. R. Knight, O. J. Sansom, et D. J. Murphy. 2018. «Calcium Signalling Links MYC to NUAK1». Oncogene 37 (8): 982-92. https://doi.org/10.1038/onc.2017.394.
Neelapu, Sattva S., Frederick L. Locke, Nancy L. Bartlett, Lazaros J. Lekakis, David B. Miklos, Caron A. Jacobson, Ira Braunschweig, et al. 2017. «Axicabtagene Ciloleucel CAR T-Cell Therapy in Refractory Large B-Cell Lymphoma». The New England Journal of Medicine 377 (26): 2531-44. https://doi.org/10.1056/NEJMoa1707447.
Nianias, Alexandros, et Maria Themeli. 2019. «Induced Pluripotent Stem Cell (IPSC)-Derived Lymphocytes for Adoptive Cell Immunotherapy: Recent Advances and Challenges». Current Hematologic Malignancy Reports 14 (4): 261-68. https://doi.org/10.1007/si 1899-019-00528-6.
Palmer, Douglas C., Beau R. Webber, Yogin Patel, Matthew J. Johnson, Christine M. Kariya, Walker S. Lahr, Maria R. Parkhurst, et al. 2020. «Internal checkpoint regulates T cell neoantigen reactivity and susceptibility to PD1 blockade». bioRxiv, janvier, 2020.09.24.306571. https://doi.org/10.1101/2020.09.24.306571.
Pazmany, L., O. Mandelboim, M. Valés-Gómez, D. M. Davis, H. T. Reyburn, et J. L. Strominger. 1996. «Protection from Natural Killer Cell-Mediated Lysis by HLA-G Expression on Target Cells». Science (New York, N. Y.) 274 (5288): 792-95. https://doi.org/10.1126/science.274.5288.792.
Poirot, Laurent, Brian Philip, Cécile Schiffer-Mannioui, Diane Le Clerre, Isabelle Chion-Sotinel, Sophie Derniame, Pierrick Potrel, et al. 2015. «Multiplex Genome-Edited T-Cell Manufacturing Platform for “Off-the-Shelf” Adoptive T-Cell Immunotherapies». Cancer Research 75 (18): 3853-64. https://doi.org/10.1158/0008-5472.CAN-14-3321.
Quezada, Sergio A., Tyler R. Simpson, Karl S. Peggs, Taha Merghoub, Jelena Vider, Xiaozhou Fan, Ronald Blasberg, et al. 2010. «Tumor-Reactive CD4(+) T Cells Develop Cytotoxic Activity and Eradicate Large Established Melanoma after Transfer into Lymphopenic Hosts». The Journal of Experimental Medicine 207 (3): 637-50. https://doi.org/10.1084/jem.20091918.
Quintarelli, Concetta, Juan F. Vera, Barbara Savoldo, Greta M. P. Giordano Attianese, Martin Pule, Aaron E. Foster, Helen E. Heslop, Cliona M. Rooney, Malcolm K. Brenner, et Gianpietro Dotti. 2007. «Co-Expression of Cytokine and Suicide Genes to Enhance the Activity and Safety of Tumor-Specific Cytotoxic T Lymphocytes». Blood 110 (8): 2793-2802. https://doi.org/10.1182/blood-2007-02-072843.
Rådestad, E., H. Wikell, M. Engström, E. Watz, B. Sundberg, S. Thunberg, M. Uzunel, J. Mattsson, et M. Uhlin. 2014. «Alpha/Beta T-Cell Depleted Grafts as an Immunological Booster to Treat Graft Failure after Hematopoietic Stem Cell Transplantation with HLA-Matched Related and Unrelated Donors». Journal of Immunology Research 2014: 578741. https://doi.org/10.1155/2014/578741.
Rajagopalan, S., et E. O. Long. 1999. «A Human Histocompatibility Leukocyte Antigen (HLA)-G-Specific Receptor Expressed on All Natural Killer Cells». The Journal of Experimental Medicine 189 (7): 1093-1100. https://doi.org/10.1084/jem.189.7.1093.
Ramanathan, Sheela, Stephanie Dubois, Julien Gagnon, Chantal Leblanc, Sanjeev Mariathasan, Gerardo Ferbeyre, Robert Rottapel, Pamela S. Ohashi, et Subburaj Ilangumaran. 2010. «Regulation of Cytokine-Driven Functional Differentiation of CD8 T Cells by Suppressor of Cytokine Signaling 1 Controls Autoimmunity and Preserves Their Proliferative Capacity toward Foreign Antigens». Journal of Immunology (Baltimore, Md.: 1950) 185 (1): 357-66. https://doi.org/10.4049/jimmunol.1000066.
Ravkov, Eugene V., et Matthew A. Williams. 2009. «The Magnitude of CD4+ T Cell Recall Responses Is Controlled by the Duration of the Secondary Stimulus». Journal of Immunology (Baltimore, Md.: 1950) 183 (4): 2382-89. https://doi.org/10.4049/jimmunol.0900319.
Ren, Bing, Hieu Cam, Yasuhiko Takahashi, Thomas Volkert, Jolyon Terragni, Richard A. Young, et Brian David Dynlacht. 2002. «E2F Integrates Cell Cycle Progression with DNA Repair, Replication, and G(2)/M Checkpoints». Genes & Development 16 (2): 245-56. https://doi.org/10.1101/gad.949802.
Ruggeri, Loredana, Marusca Capanni, Elena Urbani, Katia Perruccio, Warren D. Shlomchik, Antonella Tosti, Sabrina Posati, et al. 2002. «Effectiveness of Donor Natural Killer Cell Alloreactivity in Mismatched Hematopoietic Transplants». Science (New York, N. Y.) 295 (5562): 2097-2100. https://doi.org/10.1126/science.1068440.
Sadelain, Michel. 2015. «CAR Therapy: The CD19 Paradigm». The Journal of Clinical Investigation 125 (9): 3392-3400. https://doi.org/10.1172/JC180010.
Sckisel, Gail D., Myriam N. Bouchlaka, Arta M. Monjazeb, Marka Crittenden, Brendan D. Curti, Danice E. C. Wilkins, Kory A. Alderson, et al. 2015. «Out-of-Sequence Signal 3 Paralyzes Primary CD4(+) T-Cell-Dependent Immunity». Immunity 43 (2): 240-50. https://doi.org/10.1016/j.immuni.2015.06.023.
Seder, Robert A., et Rafi Ahmed. 2003. «Similarities and Differences in CD4+ and CD8+ Effector and Memory T Cell Generation». Nature Immunology 4 (9): 835-42. https://doi.org/10.1038/ni969.
Shifrut, Eric, Julia Carnevale, Victoria Tobin, Theodore L. Roth, Jonathan M. Woo, Christina T. Bui, P. Jonathan Li, Morgan E. Diolaiti, Alan Ashworth, et Alexander Marson. 2018. «Genome-Wide CRISPR Screens in Primary Human T Cells Reveal Key Regulators of Immune Function». Cell 175 (7): 1958-1971.e15. https://doi.org/10.1016/j.cell.2018.10.024.
Śledzińska, Anna, Maria Vila de Mucha, Katharina Bergerhoff, Alastair Hotblack, Dafne Franz Demane, Ehsan Ghorani, Ayse U. Akarca, et al. 2020a. «Regulatory T Cells Restrain Interleukin-2- and Blimp-1-Dependent Acquisition of Cytotoxic Function by CD4+ T Cells». Immunity 52 (1): 151-166.e6. https://doi.org/10.1016/j.immuni.2019.12.007.
______. 2020b. «Regulatory T Cells Restrain Interleukin-2- and Blimp-1-Dependent Acquisition of Cytotoxic Function by CD4+ T Cells». Immunity 52 (1): 151-166.e6. https://doi.org/10.1016/j.immuni.2019.12.007.
Takahashi, Reiko, Hiroko Nakatsukasa, Shunichi Shiozawa, et Akihiko Yoshimura. 2017. «SOCS1 Is a Key Molecule That Prevents Regulatory T Cell Plasticity under Inflammatory Conditions». Journal of Immunology (Baltimore, Md.: 1950) 199 (1): 149-58. https://doi.org/10.4049/jimmunol.1600441.
Takahashi, Reiko, Shuhei Nishimoto, Go Muto, Takashi Sekiya, Taiga Tamiya, Akihiro Kimura, Rimpei Morita, Mayako Asakawa, Takatoshi Chinen, et Akihiko Yoshimura. 2011. «(SOCS1 Is Essential for Regulatory T Cell Functions by Preventing Loss of Foxp3 Expression as Well as IFN-{gamma} and IL-17A Production». The Journal of Experimental Medicine 208 (10): 2055-67. https://doi.org/10.1084/jem.20110428.
Teijaro, John R., Cherie Ng, Andrew M. Lee, Brian M. Sullivan, Kathleen C. F. Sheehan, Megan Welch, Robert D. Schreiber, Juan Carlos de la Torre, et Michael B. A. Oldstone. 2013. «Persistent LCMV Infection Is Controlled by Blockade of Type I Interferon Signaling». Science (New York, N. Y.) 340 (6129): 207-11. https://doi.org/10.1126/science.1235214.
Themeli, Maria, Christopher C. Kloss, Giovanni Ciriello, Victor D. Fedorov, Fabiana Perna, Mithat Gonen, et Michel Sadelain. 2013. «Generation of Tumor-Targeted Human T Lymphocytes from Induced Pluripotent Stem Cells for Cancer Therapy». Nature Biotechnology 31 (10): 928-33. https://doi.org/10.1038/nbt.2678.
Thommen, Daniela S., et Ton N. Schumacher. 2018. «T Cell Dysfunction in Cancer». Cancer Cell 33 (4): 547-62. https://doi.org/10.1016/j.ccell.2018.03.012.
Torikai, Hiroki, Andreas Reik, Frank Soldner, Edus H. Warren, Carrie Yuen, Yuanyue Zhou, Denise L. Crossland, et al. 2013. «Toward Eliminating HLA Class I Expression to Generate Universal Cells from Allogeneic Donors». Blood 122 (8): 1341-49. https://doi.org/10.1182/blood-2013-03-478255.
Vacchio, Melanie S., et Richard J. Hodes. 2005. «Fetal Expression of Fas Ligand Is Necessary and Sufficient for Induction of CD8 T Cell Tolerance to the Fetal Antigen H-Y during Pregnancy». Journal of Immunology (Baltimore, Md.: 1950) 174 (8): 4657-61. https://doi.org/10.4049/jimmunol.174.8.4657.
Valton, Julien, Valérie Guyot, Alan Marechal, Jean-Marie Filhol, Alexandre Juillerat, Aymeric Duclert, Philippe Duchateau, et Laurent Poirot. 2015. «A Multidrug-Resistant Engineered CAR T Cell for Allogeneic Combination Immunotherapy». Molecular Therapy: The Journal of the American Society of Gene Therapy 23 (9): 1507-18. https://doi.org/10.1038/mt.2015.104.
Wang, Dachun, Yuan Quan, Qing Yan, John E. Morales, et Rick A. Wetsel. 2015. «Targeted Disruption of the B2-Microglobulin Gene Minimizes the Immunogenicity of Human Embryonic Stem Cells». Stem Cells Translational Medicine 4 (10): 1234-45. https://doi.org/10.5966/sctm.2015-0049.
Wang, Tao, Catherine Huang, Alfonso Lopez-Coral, Kimberly A. Slentz-Kesler, Min Xiao, E. John Wherry, et Russel E. Kaufman. 2012. «K12/SECTM1, an Interferon-γ Regulated Molecule, Synergizes with CD28 to Costimulate Human T Cell Proliferation». Journal of Leukocyte Biology 91 (3): 449-59. https://doi.org/10.1189/jlb.1011498.
Wei, Jun, Lingyun Long, Wenting Zheng, Yogesh Dhungana, Seon Ah Lim, Cliff Guy, Yanyan Wang, et al. 2019. «Targeting REGNASE-1 Programs Long-Lived Effector T Cells for Cancer Therapy». Nature 576 (7787): 471-76. https://doi.org/10.1038/s41586-019-1821-z.
Wherry, E. John, et Makoto Kurachi. 2015. «Molecular and Cellular Insights into T Cell Exhaustion». Nature Reviews. Immunology 15 (8): 486-99. https://doi.org/10.1038/nri3862.
Wilson, Elizabeth B., Douglas H. Yamada, Heidi Elsaesser, Jonathan Herskovitz, Jane Deng, Genhong Cheng, Bruce J. Aronow, Christopher L. Karp, et David G. Brooks. 2013. «Blockade of Chronic Type I Interferon Signaling to Control Persistent LCMV Infection». Science (New York, N. Y.) 340 (6129): 202-7. https://doi.org/10.1126/science.1235208.
Xie, Ying, Akguil Akpinarli, Charles Maris, Edward L. Hipkiss, Malcolm Lane, Eun-Kyung M. Kwon, Pawel Muranski, Nicholas P. Restifo, et Paul Andrew Antony. 2010. «Naive Tumor-Specific CD4(+) T Cells Differentiated in Vivo Eradicate Established Melanoma». The Journal of Experimental Medicine 207 (3): 651-67. https://doi.org/10.1084/jem.20091921.
Zhou, Penghui, Donald R. Shaffer, Diana A. Alvarez Arias, Yukoh Nakazaki, Wouter Pos, Alexis J. Torres, Viviana Cremasco, et al. 2014. «In Vivo Discovery of Immunotherapy Targets in the Tumour Microenvironment». Nature 506 (7486): 52-57. https://doi.org/10.1038/nature12988.
Zhu, J. W., S. J. Field, L. Gore, M. Thompson, H. Yang, Y. Fujiwara, R. D. Cardiff, M. Greenberg, S. H. Orkin, et J. DeGregori. 2001. «E2F1 and E2F2 Determine Thresholds for Antigen-Induced T-Cell Proliferation and Suppress Tumorigenesis». Molecular and Cellular Biology 21 (24): 8547-64. https://doi.org/10.1128/MCB.21.24.8547-8564.2001.
Zhu, Ziqiang, Steven M. Cuss, Vinod Singh, Devikala Gurusamy, Jennifer L. Shoe, Robert Leighty, Vincenzo Bronte, et Arthur A. Hurwitz. 2015. «CD4+ T Cell Help Selectively Enhances High-Avidity Tumor Antigen-Specific CD8+ T Cells». Journal of Immunology (Baltimore, Md.: 1950) 195 (7): 3482-89. https://doi.org/10.4049/jimmunol.1401571.

1-16. (canceled)

17. An engineered immune cell, which is defective for SOCS-1.

18. The engineered immune cell of claim 17, which is further defective for at least one additional protein, particularly FAS, Suv39h1 and or β2m, optionally wherein the cell is defective at least for SOCS1 and FAS.

19. The engineered immune cell according to claim 17, which further comprises a genetically engineered antigen receptor that specifically binds a target antigen.

20. The engineered immune cell according to claim 17, which is a T cell or an NK cell.

21. The engineered immune cell of claim 17, which is a CD4+ or CD8+ T cell.

22. The engineered immune cell according to claim 17, which is isolated from a subject.

23. The engineered immune cell according to claim 22, wherein the subject is suffering from a cancer, or is at risk of suffering from a cancer.

24. The engineered immune cell according to claim 17, wherein the activity and/or expression of SOCS-1, SOCS1 and FAS, of SOCS-1 and Suv39h1, or SOCS1, Suv39h1 and FAS in the said engineered immune cell is selectively inhibited or blocked.

25. The engineered immune cell according to claim 17, wherein said engineered immune cell expresses a SOCS-1 nucleic acid encoding a non-functional SOCS-1 protein and optionally wherein said engineered immune cell further expressed a Suv39h1 nucleic acid encoding a non-functional Suv39h1 protein, a FAS nucleic acid encoding a non-functional FAS protein and/or a β2m nucleic acid encoding a non-functional β2m protein.

26. The engineered immune cell according to claim 17, wherein the target antigen is expressed by cancer cells and/or is a universal tumor antigen.

27. The engineered immune cell according to claim 19, wherein the genetically engineered antigen receptor is a chimeric antigen receptor (CAR) comprising an extracellular antigen-recognition domain that specifically binds to the target antigen, or a TCR.

28. The cell of claim 18, wherein the genetically engineered antigen receptor is a T cell receptor (TCR).

29. A method of producing a universal genetically engineered immune cell comprising
inhibiting the expression and/or activity of SOCS1 and/or FAS in the immune cell; and optionally comprising:
inhibiting the expression and/or activity of Suv39h1 and/or β2m in the immune cell, and/or
optionally introducing in the said immune cell a genetically engineered antigen receptor that specifically binds to a target antigen.

30. The method of claim 29, wherein the inhibition of SOCS1, FAS, Suv39h1 or β2m expression and/or activity comprises putting in contact the cell with at least an agent inhibiting the expression and/or activity of respectively SOCS1, FAS, Suv39h1 or β2m and/or respectively disrupting the SOCS1, FAS, Suv39h1 or B2M gene.

31. The method of claim 30, wherein the agent is selected from small molecule inhibitors; antibodies derivatives, aptamers, nucleic acid molecules that block transcription or translation, or gene editing agents.

32. A method of adoptive cellular therapy for treatment of cancer, comprising administering to a subject in need thereof at least one engineered immune cell according to claim 17.

33. The method according to claim 32, which is for allogenic cellular therapy of cancer.


SEQUENCE LISTING (5 sequences)

SEQ ID NO: 1 - protein, 211 aa, Homo sapiens
MVAHNQVAADNAVSTAAEPRRRPEPSSSSSSSPAAPARPRPCPAVPAPAPGDTHFRTFRSHADYRRITRASALLDACGFYWGPLSVHGAHERLRAEPVGTFLVRDSRQRNCFFALSVKMASGPTSIRVHFQAGRFHLDGSRESFDCLFELLEHYVAAPRRMLGAPLRQRRVRPLQELCRQRIVATVGRENLARIPLNPVLRDYLSSFPFQI

SEQ ID NO: 2 - protein, 412 aa, Homo sapiens
MAENLKGCSVCCKSSWNQLQDLCRLAKLSCPALGISKRNLYDFEVEYLCDYKKIREQEYYLVKWRGYPDSESTWEPRQNLKCVRILKQFHKDLERELLRRHHRSKTPRHLDPSLANYLVQKAKQRRALRRWEQELNAKRSHLGRITVENEVDLDGPPRAFVYINEYRVGEGITLNQVAVGCECQDCLWAPTGGCCPGASLHKFAYNDQGQVRLRAGLPIYECNSRCRCGYDCPNRVVQKGIRYDLCIFRTDDGRGWGVRTLEKIRKNSFVMEYVGEIITSEEAERRGQIYDRQGATYLFDLDYVEDVYTVDAAYYGNISHFVNHSCDPNLQVYNVFIDNLDERLPRIAFFATRTIRAGEELTFDYNMQVDPVDMESTRMDSNFGLAGLPGSPKKRVRIECKCGTESCRKYLF

SEQ ID NO: 3 - protein, 423 aa, Homo sapiens
MVGMSRLRNDRLADPLTGCSVCCKSSWNQLQDLCRLAKLSCPALGISKRNLYDFEVEYLCDYKKIREQEYYLVKWRGYPDSESTWEPRQNLKCVRILKQFHKDLERELLRRHHRSKTPRHLDPSLANYLVQKAKQRRALRRWEQELNAKRSHLGRITVENEVDLDGPPRAFVYINEYRVGEGITLNQVAVGCECQDCLWAPTGGCCPGASLHKFAYNDQGQVRLRAGLPIYECNSRCRCGYDCPNRVVQKGIRYDLCIFRTDDGRGWGVRTLEKIRKNSFVMEYVGEIITSEEAERRGQIYDRQGATYLFDLDYVEDVYTVDAAYYGNISHFVNHSCDPNLQVYNVFIDNLDERLPRIAFFATRTIRAGEELTFDYNMQVDPVDMESTRMDSNFGLAGLPGSPKKRVRIECKCGTESCRKYLF

SEQ ID NO: 4 - protein, 335 aa, Homo sapiens
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRKEVQKTCRKHRKENQGSHESPTLNPETVAINLSDVDLSKYITTIAGVMTLSQVKGFVRKNGVNEAKIDEIKNDNVQDTAEQKVQLLRNWHQLHGKKEAYDTLIKDLKKANLCTLAEKIQTIILKDITSDSENSNFRNEIQSLV

SEQ ID NO: 5 - protein, 119 aa, Homo sapiens
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM